(19) United States
(12) Patent Application Publication
(10) Pub. No.: US 2024/0294907 A1
(43) Pub. Date: Sep. 5, 2024

(54) SELECTIVE DELIVERY OF OLIGONUCLEOTIDES TO GLIAL CELLS

(71) Applicant: Dicerna Pharmaceuticals, Inc., Lexington, MA (US)

(72) Inventors: Maire JUNG, Lincoln, MA (US); Travis GRIM, Maynard, MA (US)

(21) Appl. No.: 18/030,230

(22) PCT Filed: Oct. 8, 2021
(86) PCT No.: PCT/US2021/071785

§ 371 Date: Apr. 4, 2023

Related U.S. Application Data
(60) Provisional application No. 63/089,406, filed on Oct. 8, 2020.

(51) Int. Cl. C12N 15/113

(52) U.S. Cl. CPC C12N 15/113; C12N 2310/14; C12N 2310/351; C12N 2310/531; C12N 2320/32

(57) ABSTRACT

The present disclosure relates to oligonucleotides for selective reduction of levels of target RNA expression and/or proteins expressed in glial cells, and related methods of delivering such oligonucleotides into glial cells.

### CROSS-REFERENCE TO RELATED APPLICATIONS

The present application is a § 371 National Stage of PCT International Application No. PCT/US2021/071785, filed Oct. 8, 2021, which claims the benefit under 35 U.S.C. § 119 (e) of U.S. Provisional Application No. 63/089,406, filed Oct. 8, 2020, each of which is incorporated herein by reference in its entirety.

### SEQUENCE LISTING

The instant application contains a Sequence Listing which has been submitted in ASCII format and is hereby incorporated by reference in its entirety. The ASCII copy, created on Nov. 5, 2021, is named “Sequence Listing.txt” and is 3.5 kilobytes in size.

### BACKGROUND

The nervous system consists of neurons and glial cells. Neurons function primarily in generating and propagating chemical and electrical signals. Glial cells function to modulate neuron function and signaling, and thereby sculpt and modulate neuronal properties and function. In the central nervous system (CNS), glial cells include astrocytes, oligodendrocytes, ependymal cells, and microglia. In the peripheral nervous system, the main glial cells include Schwann cells, enteric glial cells, and satellite cells.

Glial cells are implicated in a variety of disease processes. Several neurodegenerative diseases may result from faulty glial cell function. For example, astrocyte dysfunction may be involved in amyotrophic lateral sclerosis (ALS), Huntington's Disease, and Parkinson's Disease. Oligodendrocytes, which function to form protective sheaths on axons of nerve cells, may be involved in gliomas, schizophrenia, bipolar disorder, and leukodystrophies. Microglia have a role in immune function in the brain and invokes inflammatory responses that may promote Alzheimer's Disease, autism, and schizophrenia. Malfunctioning of Schwann cells are associated with Guillain-Barre' syndrome, Charcot-Marie-Tooth disease, and chronic inflammatory demyelinating polyneuropathy.

Some diseases involving glial cell dysfunction are associated with expression of certain gene products. For example, Alexander's disease is a form of leukodystrophy characterized by destruction of the myelin sheath and is caused by autosomal gain of function mutations in the gene for glial fibrillary acid protein (GFAP), an intermediate filament protein expressed in astrocytes. The overexpression and accumulation of GFAP results in abnormal protein deposits known as Rosenthal fibers, which may impair formation of normal intermediate fibers.

Another disease associated with expression of an abnormal gene product in glial cells is ALS. ALS is associated with mutations in the gene encoding copper-zinc superoxide dismutase 1 (SOD1). In mouse models of ALS, selective expression in motor neurons of mutated SOD1 (mSOD1) alone is insufficient to cause full ALS symptoms (see Pramatarova et al., J NEUROSCI. 2001, 21 (10):3369-74). However, selective depletion of mSOD1 in motor neurons affects onset and survival but not disease progression, suggesting the involvement of surrounding glial cells in influencing degeneration.

In view of the numerous diseases and disorders associated with dysfunction in glial cells, desirable are therapeutic approaches for selectively targeting glial cells through modulating expression of gene products involved in disease or disorder associated with glial cell function.

### SUMMARY

The present disclosure relates to the surprising discovery that administration of an interfering oligonucleotide (e.g. RNAi oligonucleotide) into a mammalian nervous system results in selective reduction in levels of mRNA expressed in glial cells as compared to reduction in expression of an mRNA expressed in neuronal cells. The current invention provides that the selective uptake in glial cells and/or selective reduction in levels of RNA expressed in glial cells could be achieved with or without the presence of GalNAc targeting moieties on the oligonucleotides.

Accordingly, in one aspect, the present disclosure provides a method of selective delivery of an interfering oligonucleotide to glial cells, in particular where the selectivity is for glial cells over neuronal cells. In some embodiments, a method of selective delivery of an interfering oligonucleotide comprises contacting a glial cell with an interfering oligonucleotide having a region of complementary to a target RNA, wherein the oligonucleotide is capable of reducing expression of the target RNA.

In another aspect, the present disclosure provides a method of selectively reducing levels of an RNA and/or a protein encoded by the RNA expressed in glial cells. In some embodiments, a method of selectively reducing levels of a RNA and/or a protein encoded by the RNA expressed in glial cells comprises contacting a glial cell with an oligonucleotide having a region of complementarity to the target RNA, wherein the oligonucleotide is capable of reducing expression of the target RNA.

In some embodiments, the region of complementarity is to an exon of a target RNA. In some embodiments, the region of complementarity is to a 5′-untranslated region of a target RNA. In some embodiments, the region of complementarity is to a 3′-untranslated region of a target RNA. In some embodiments, the region of complementarity is to an allele specific sequence of a target RNA, such as a polymorphic sequence.

In some embodiments, the oligonucleotide is 12 to 60 nucleotides in length. In some embodiments, the oligonucleotide has a region of complementarity to the target RNA of at least 12 nucleotides in length. In some embodiments, the region of complementarity to the target RNA of 12 to 30 nucleotides in length.

In some embodiments, the oligonucleotide is single stranded. In some embodiments, the oligonucleotide comprises a double stranded nucleic acid (dsNA). In some embodiments, the oligonucleotide comprises RNA.

In some embodiments, the oligonucleotide comprises a sense strand and an antisense strand, wherein the sense strand and the antisense strand form a duplex, wherein the antisense strand comprises a region of complementarity to a target RNA expressed in a glial cell and is capable of reducing levels of the target RNA.

In some embodiments, the sense strand is 15 to 40 nucleotides in length. In some embodiments, the antisense strand is 19 to 27 nucleotides in length. In some embodiments, the sense strand and the antisense strand form a duplex of at least 12 nucleotides in length. In some embodiments, the sense strand and the antisense strand form a duplex of 12 to 30 nucleotides in length.

In some embodiments, the sense strand and the antisense strand are separate oligonucleotides. In some embodiments, the sense strand and the antisense strand comprise a single oligonucleotide.

In some embodiments, the sense strand or the antisense strand has a 3′-overhang of up to two nucleotides in length when the sense strand and the antisense strand form a duplex. Preferably, the antisense strand has a 3′-overhang of up to 2 nucleotides in length.

In some embodiments, the oligonucleotide further comprises a stem-loop sequence comprising sequence regions S1-L-S2, wherein S1 is complementary to S2, and wherein L is a loop that forms between S1 and S2 when S1 and S2 form a duplex, and wherein the stem-loop sequence is attached to the 3′-end of the sense strand. In some embodiments, the loop L is a pentaloop, tetraloop or a triloop.

In some embodiments, the oligonucleotide further comprises one or more targeting ligands or targeting moieties. In some embodiments, the target ligand or moiety is present on the loop L of a stem-loop sequence of an oligonucleotide. In some embodiments, the targeting ligand or moiety is a GalNAc moiety. In some embodiments, the oligonucleotide has no GalNAc moiety.

In some embodiments, at least one nucleotide of the oligonucleotide is modified. In some embodiments, one or more of the nucleotides of the oligonucleotide are modified. In some embodiments, the modifications include modifications of the sugar moiety, internucleoside linkage, 5′-terminal phosphate, nucleotide base, reversible modifications, and as discussed above, one or more targeting moieties.

In some embodiments, the glial cell for targeting with the oligonucleotide is an astrocyte, oligodendrocyte, ependymal cell, microglial cell, Schwann cell, satellite cell, enteric glial cell, or mixtures thereof. In some embodiments, the glial cell is present in the central nervous system of a subject. In some embodiments, the glial cell is present in the peripheral nervous system of a subject. In some embodiments, the glial cell for targeting is present in specified regions of the central nervous system, for example, the frontal cortex, striatum, somatosensory cortex, hippocampus, hypothalamus, cerebellum, brainstem, and/or spinal cord. In some embodiments, the glial cell is present in specified regions of the spinal cord, such as the cervical spinal cord, thoracic spinal cord, or lumbar spinal cord.

In some embodiments, the selectivity for delivery to a glial cell or selectivity for reduction in levels of an RNA expressed in a glial cell is in comparison to a neuronal cell. In some embodiments, the selective delivery or selective reduction in levels of an RNA expressed in a glial cell is at least 1.5-fold or more for glial cell over neuronal cells. In some embodiments, the selective delivery or selective reduction in levels of an RNA expressed in a glial cell is 2 or greater; 2.5 or greater; 3 or greater; 3.5 or greater; 4 or greater; 4.5 or greater; or 5 or greater for glial cell over neuronal cell.

In some embodiments, the method of selective delivery of an interfering oligonucleotide or selective reduction in levels of a RNA expressed in a glial cell is used to treat a disorder or condition associated with a dysfunction of glial cells, such as dysfunction of astrocytes, oligodendrocytes, ependymal cells, microglial cells, Schwann cells, satellite cells, and/or enteric glial cells.

In some embodiments, the selective delivery or selective reduction in levels of an RNA expressed in a glial cell is by intrathecal, intraventrical, interstitial, sublingual, intravenous, or intranasal administration to a subject an effective amount an oligonucleotide described herein.

### BRIEF DESCRIPTION OF FIGURES

The accompanying drawings, which are incorporated herein and constitute a part of this specification, illustrate certain embodiments, and together with the written description, serve to provide non-limiting examples of certain aspects of the compositions and methods disclosed herein.

FIG. 1 shows the structures of the siRNA targeting GFAP and TUBB3 mRNAs. The tetraloop sequence GAAA was modified with GalNAc moieties.

FIGS. 2A and 2B show cell type specific Tubb3 and Gfap mRNA expression throughout the CNS of mice.

FIG. 3 shows GalXC-GFAP 30-day screen dose response at doses of 0.3 mg/kg, 1 mg/kg, and 3 mg/kg single subcutaneous dose, HDI 2 days followed by harvest 4 days post subcutaneous dose.

FIG. 4 shows GalXC-TUBB3 30-day screen dose response at doses of 0.3 mg/kg, 1 mg/kg, and 3 mg/kg single subcutaneous dose, HDI 2 days followed by harvest 4 days post subcutaneous dose.

FIG. 5 shows GalXC siRNA pharmacology of astrocyte specific Gfap mRNA silencing and neuron specific Tubb3 mRNA silencing in the frontal cortex.

FIG. 6 shows GalXC siRNA pharmacology of astrocyte specific Gfap mRNA silencing and neuron specific Tubb3 mRNA silencing in the striatum.

FIG. 7 shows GalXC siRNA pharmacology of astrocyte specific Gfap mRNA silencing and neuron specific Tubb3 mRNA silencing in the somatosensory cortex.

FIG. 8 shows GalXC siRNA pharmacology of astrocyte specific Gfap mRNA silencing and neuron specific Tubb3 mRNA silencing in the hippocampus.

FIG. 9 shows GalXC siRNA pharmacology of astrocyte specific Gfap mRNA silencing and neuron specific Tubb3 mRNA silencing in the hypothalamus.

FIG. 10 shows GalXC siRNA pharmacology of astrocyte specific Gfap mRNA silencing and neuron specific Tubb3 mRNA silencing in the cerebellum.

FIG. 11 shows GalXC siRNA pharmacology of astrocyte specific Gfap mRNA silencing and neuron specific Tubb3 mRNA silencing in the brainstem.

FIG. 12 shows GalXC siRNA pharmacology of neuron specific Tubb3 mRNA silencing in the cervical spinal cord, thoracic spinal cord, and lumbar spinal cord.

FIG. 13 shows GalXC siRNA pharmacology of astrocyte specific Gfap mRNA silencing or neuron specific Tubb3 mRNA silencing in lumbar spinal cord, where the siRNAs are either modified with GalNAc residues or are not modified.

FIG. 14 shows the structures of exemplary oligonucleotides with the modification patterns comprising a sense strand of 36 nucleotides in length and an antisense of 22 nucleotides in length, where the sense strand contains a stem-loop sequence comprising regions S1-L-S2.

FIG. 15 shows the structure of an exemplary oligonucleotide with the modification pattern comprising a sense strand of 36 nucleotides in length and an antisense of 22 nucleotides in length, where the sense strand contains a stem-loop sequence comprising regions S1-L-S2.

### DETAILED DESCRIPTION

The present disclosure relates to compositions and uses of the compositions for selective delivery of an interfering oligonucleotide and to selective reduction in levels of an RNA expressed in glial cells, including selective delivery to or selective reduction in levels of a target RNA expressed in glial cells in the nervous system of a subject. The disclosure presents unexpected finding of selective delivery and selective reduction in levels of an RNA expressed in glial cells as compared to other neuronal cells using the RNAi oligonucleotides disclosed herein. Accordingly, the following provides a detailed description of oligonucleotide compositions and selective delivery of the oligonucleotides to glial cells and uses of the oligonucleotides for selective reduction in levels of a target RNA and/or the protein encoded by the RNA expressed in glial cells, such as for treating diseases or disorders associated with glial cell dysfunction.

It is to be understood that both the foregoing general description, including the drawings, and the following detailed description are exemplary and explanatory only and are not restrictive of this disclosure.

The section headings used herein are for organizational purposes only and not to be construed as limiting the subject matter described.

### I. Definitions

In reference to the present disclosure, the technical and scientific terms used in the descriptions herein will have the meanings commonly understood by one of ordinary skill in the art, unless specifically defined otherwise. Accordingly, the following terms are intended to have the following meanings.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Administering: As used herein, the terms “administering” or “administration” means to provide a substance (e.g., an oligonucleotide) to a subject in a manner that is pharmacologically useful (e.g., to treat a condition in the subject).

Complementary: As used herein, the term “complementary refers to a structural relationship between nucleotides (e.g., two nucleotide on opposing nucleic acids or on opposing regions of a single nucleic acid strand) that permits the nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have nucleotide sequences that are complementary to each other to form regions of complementarity, as described herein.

Deoxyribonucleotide: As used herein, the term “deoxyribonucleotide” refers to a nucleotide having a hydrogen at the 2′ position of its pentose sugar as compared with a ribonucleotide. A modified deoxyribonucleotide is a deoxyribonucleotide having one or more modifications or substitutions of atoms other than at the 2′ position, including modifications or substitutions in or of the sugar, phosphate group or base.

Double-stranded oligonucleotide: As used herein, the term “double-stranded oligonucleotide” refers to an oligonucleotide that is substantially in a duplex form. In some embodiments, complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from a single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed, e.g., having overhangs at one or both ends. In some embodiments, a double-stranded oligonucleotide comprises antiparallel sequences of nucleotides that are partially complementary, and thus, may have one or more mismatches, which may include internal mismatches or end mismatches.

Duplex: As used herein, the term “duplex,” in reference to nucleic acids (e.g., oligonucleotides), refers to a structure formed through complementary base-pairing of two antiparallel sequences of nucleotides.

Excipient: As used herein, the term “excipient” refers to a non-therapeutic agent that may be included in a composition, for example, to provide or contribute to a desired consistency or stabilizing effect.

Glial Cell: As used herein, “glial cell” and “neuroglial cells” are used interchangeably herein and refer to non-neuronal precursor and/or fully differentiated cells in the nervous system that provide support or nutrition or work to maintain homeostasis. Examples of glial cells include, among others, ependymal cells, oligodendrocytes, astrocytes, microglial cells, Schwann cells, satellite cells, and enteric glial cells. Glial cells have also been found to regulate nerve firing rates, brain plasticity, and immune responses.

Loop: As used herein, the term “loop” to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

Modified Internucleotide Linkage: As used herein, the term “modified internucleotide linkage” refers to an internucleotide linkage having one or more chemical modifications compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

Modified Nucleotide: As used herein, the term “modified nucleotide” refers to a nucleotide having one or more chemical modifications compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modifications in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc. In certain embodiments, a modified nucleotide comprises a 2′-O-methyl or a 2′-F substitution at the 2′ position of the ribose ring.

Nicked Tetraloop Structure: A “nicked tetraloop structure” refers to a structure of a RNAi oligonucleotide characterized by the presence of separate sense (passenger) and antisense (guide) strands, in which the sense strand has a region of complementarity to the antisense strand such that the two strands form a duplex, and in which at least one of the strands, generally the sense strand, extends from the duplex in which the extension contains a tetraloop and two self-complementary sequences forming a stem region adjacent to the tetraloop, in which the tetraloop is configured to stabilize the adjacent stem region formed by the self-complementary sequences of the at least one strand.

Neuronal Cell: A “neuronal cell” refers generally to the structural and functional units of the nervous system and are present in the central and peripheral nervous system. Neuronal cells function as the conducting cells of the nervous system, receiving and transmitting chemical and/or electrical signals. Three general categories of neuronal cells include sensory neurons, motor neurons, and interneurons. In some embodiments, neuronal cells can be distinguished from non-neuronal glial cells by the expression of specific neuronal specific markers, including, by way of example and not limitation, neuron specific enolase (NSE or gamma-enolase); neuronal nuclei (NeuN or Fox3); microtubule-associated protein 2 (MAP-2); Tubulin beta III (TUBB3); Doublecortin (DCX); and c-fos. In some embodiments, clinical markers for neuronal cell include choline acetyltransferase (ChAT) and tyrosine hydroxylase. Other neuronal cell markers specific to regions of the central nervous system or peripheral nervous system can also be used, e.g., calbindin-D28K, calretinin, and neurofilament protein (NFP).

Oligonucleotide: As used herein, the term “oligonucleotide” refers to a short nucleic acid, e.g., of less than 100 nucleotides in length. An oligonucleotide can comprise ribonucleotides, deoxyribonucleotides, and/or modified nucleotides including, for example, modified ribonucleotides. An oligonucleotide may be single-stranded or double-stranded. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA, or single-stranded siRNA. In some embodiments, a double-stranded oligonucleotide is an RNAi oligonucleotide.

Overhang: As used herein, the term “overhang” refers to terminal non-base-pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a double-stranded oligonucleotide. In certain embodiments, the overhang is a 3′ or 5′ overhang on the antisense strand or sense strand of a double-stranded oligonucleotide.

Phosphate analog: As used herein, the term “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof (see, e.g., International patent publication WO/2018/045317, the contents of which relating to phosphate analogs are incorporated herein by reference. Other modifications have been developed for the 5′ end of oligonucleotides (see, e.g., International patent publication WO2011133871; U.S. Pat. No. 8,927,513; and Prakash et al., Nucleic Acids Res., 2015, 43 (6):2993-3011, the contents of each of which relating to phosphate analogs are incorporated herein by reference).

Reduced expression: As used herein, the term “reduced expression” or equivalents thereof of a gene refers to a decrease in the amount of RNA transcript or protein encoded by the gene and/or a decrease in the amount of activity of the gene in a cell or subject, as compared to an appropriate reference cell or subject. For example, the act of treating a cell with a double-stranded oligonucleotide (e.g., one having an antisense strand that is complementary to target mRNA sequence) may result in a decrease in the amount of RNA transcript, protein and/or enzymatic activity (e.g., encoded by the target gene) compared to a cell that is not treated with the double-stranded oligonucleotide. Similarly, “reducing expression” as used herein refers to an act that results in reduced expression of a gene (e.g., a target gene).

Region of Complementarity: As used herein, the term “region of complementarity” refers to a sequence of nucleotides of a nucleic acid (e.g., a double-stranded oligonucleotide) that is sufficiently complementary to an antiparallel sequence of nucleotides (e.g., a target nucleotide sequence within an mRNA) to permit hybridization between the two sequences of nucleotides under appropriate hybridization conditions, e.g., in a phosphate buffer, in a cell, etc. A region of complementarity may be fully complementary to a nucleotide sequence (e.g., a target nucleotide sequence present within an mRNA or portion thereof). For example, a region of complementary that is fully complementary to a nucleotide sequence present in an mRNA has a contiguous sequence of nucleotides that is complementary, without any mismatches or gaps, to a corresponding sequence in the mRNA. Alternatively, a region of complementarity may be partially complementary to a nucleotide sequence (e.g., a nucleotide sequence present in an mRNA or portion thereof). For example, a region of complementary that is partially complementary to a nucleotide sequence present in an mRNA has a contiguous sequence of nucleotides that is complementary to a corresponding sequence in the mRNA but that contains one or more mismatches or gaps (e.g., 1, 2, 3, or more mismatches or gaps) compared with the corresponding sequence in the mRNA, provided that the region of complementarity remains capable of hybridizing with the mRNA under appropriate hybridization conditions.

Ribonucleotide: As used herein, the term “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

RNAi Oligonucleotide: As used herein, the term “RNAi oligonucleotide” refers to either (a) a double stranded oligonucleotide having a sense strand (passenger) and antisense strand (guide), in which the antisense strand or part of the antisense strand is used by the Argonaute 2 (Ago2) endonuclease in the cleavage of a target mRNA or (b) a single stranded oligonucleotide having a single antisense strand, where that antisense strand (or part of that antisense strand) is used by the Ago2 endonuclease in the cleavage of a target mRNA.

Selective reduction: As used herein, a “selective reduction” or equivalents thereof in expression of a gene refers to a preferential decrease in the amount of RNA transcript or protein encoded by the gene and/or a decrease in the amount of activity of the gene in a cell of interest as compared to an appropriate reference cell or subject, where the gene in the cell of interest and the comparator gene in the reference cell or subject is the same gene or different gene. In some embodiments, the expression of the gene in the cell of interest is specific to the cell of interest and the expression of the comparator gene in the reference cell is specific to the reference cell.

Strand: As used herein, the term “strand” refers to a single contiguous sequence of nucleotides linked together through internucleotide linkages (e.g., phosphodiester linkages, phosphorothioate linkages). In some embodiments, a strand has two free ends, e.g., a 5′-end and a 3′-end.

Subject: As used herein, the term “subject” means any mammal, including mice, rabbits, and humans. In some embodiments, the subject is a human or non-human primate. The terms “individual” or “patient” may be used interchangeably with “subject.”

Synthetic: As used herein, the term “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

Targeting ligand: As used herein, the term “targeting ligand” refers to a molecule (e.g., a carbohydrate, amino sugar, cholesterol, polypeptide or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

Tetraloop: As used herein, the term “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. In some embodiments, a tetraloop may stabilize a base pair in an adjacent stem duplex by stacking interactions. In addition, interactions among the nucleotides in a tetraloop include but are not limited to non-Watson-Crick base-pairing, stacking interactions, hydrogen bonding, and contact interactions (Cheong et al., NATURE, 1990, 346 (6285):680-2; Heus and Pardi, SCIENCE, 1991, 253 (5016):191-4). In some embodiments, a tetraloop comprises or consists of 3 to 6 nucleotides and is typically 4 to 5 nucleotides. In certain embodiments, a tetraloop comprises or consists of three, four, five, or six nucleotides, which may or may not be modified (e.g., which may or may not be conjugated to a targeting moiety). In some embodiments, a tetraloop consists of four nucleotides. Any nucleotide may be used in the tetraloop and standard IUPAC-IUB symbols for such nucleotides may be used as described in Cornish-Bowden, NUCLEIC ACIDS RES. 1985, 13:3021-3030. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al., PROC NATL ACAD SCI. USA., 1990, 87 (21):8467-71; Antao et al., NUCLEIC ACIDS RES., 1991, 19 (21):5901-5). Examples of DNA tetraloops include the d(GNNA) family of tetraloops (e.g., d(GTTA)), the d(GNRA) family of tetraloops, the d(GNAB) family of tetraloops, the d(CNNG) family of tetraloops, and the d(TNCG) family of tetraloops (e.g., d(TTCG)). See, for example: Nakano et al., BIOCHEMISTRY, 2002, 41 (48):14281-292; Shinji et al., Nippon KAGAKKAI KOEN YOKOSHU, 2000, 78 (2):731, which are incorporated by reference herein for their relevant disclosures. In some embodiments, the tetraloop is contained within a nicked tetraloop structure.

Treat, treatment, or treating: As used herein, the term “treat,” “treatment,” or “treating” refers to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

### II. Oligonucleotides for Selective Delivery and Reduction of RNA and/or Protein Expression in Glial Cells

The present disclosure relates to compositions and methods for selective delivery of interfering oligonucleotides or selective reduction in levels of a target RNA and/or protein expressed in glial cells, in particular to selective delivery to or selective reduction in levels of a target RNA and/or protein expressed in glial cells in the nervous system of a subject. As discussed above, it is shown herein that administration of an interfering RNA (RNAi) modified with GalNAc residues into the nervous system results in surprising selective reduction of levels of a target mRNA specifically expressed in glial cells as compared to reduction in levels of a target mRNA specifically expressed in neuronal cells. In some instances, the selectivity for reduction of a mRNA expressed in glial cells is nearly two-fold (e.g., over 80% reduction) over reduction in expression of an mRNA expressed in neuronal cells (e.g., about 40% reduction). While it is known that GalNAc ligands on the oligonucleotide target it for selective uptake in the liver, the results in the present application show that such modified oligonucleotides selectively reduce levels of a target RNA expressed in glial cells over reduction in levels of a target RNA in neuronal cells. The known specificity of GalNAc for hepatic asialoglycoprotein receptors and the contrasting selective activity of GalNAc modified oligonucleotides shows that selective uptake in glial cells and/or selective reduction in levels of RNA expressed in glial cells may be achieved without the presence of the GalNAc ligands.

### a. Oligonucleotides

In some embodiments, a method of selective delivery of an interfering oligonucleotide or a method of selectively reducing levels of a target RNA expressed in glial cells comprises contacting a glial cell with an oligonucleotide capable of reducing levels of an RNA and/or protein expressed in the glial cell.

In some embodiments, the oligonucleotide comprises a region of complementarity to a target RNA expressed in a glial cell. In some embodiments, the region of complementarity is at least 12 nucleotides in length. In some embodiments, the region of complementarity is at least 15 nucleotides in length. In some embodiments, the region of complementarity is at least 19 nucleotides in length. In some embodiments, the region of complementarity is 12 to 30 nucleotides in length. In some embodiments, the region of complementarity is 19 to 23 nucleotides in length.

In some embodiments, the oligonucleotide is at least 12 nucleotides in length. In some embodiments, the oligonucleotide is 12 to 60 nucleotides in length. In some embodiments, the oligonucleotide is 12 to 58 nucleotides in length.

In some embodiments, the oligonucleotide is a single stranded nucleic acid. In some embodiments, the oligonucleotide is a double stranded nucleic acid (dsNA).

In some embodiments, the oligonucleotide comprises a sense strand and an antisense strand, wherein the sense strand and antisense strand are capable of forming a duplex, and the antisense strand comprises a region of complementary to a target RNA sequence expressed in a glial cell. In some embodiments, the duplex formed by the sense strand and the antisense strand is referred to as the first duplex (D1). In some embodiments, the antisense strand can reduce levels of target RNA expressed in glial cells.

In some embodiments, the sense strand is 15 to 40 nucleotides in length. In some embodiments, the sense strand is 19 to 40 nucleotides in length. In some embodiments, the sense strand is 12 to 36 nucleotides in length. In some embodiments, the sense strand is 17 to 36 nucleotides in length. In some embodiments, the sense strand is 19 to 23 nucleotides in length.

In some embodiments, the antisense strand is up to 50 nucleotides in length (e.g., up to 40, up to 30, up to 27, up to 25, up to 21, or up to 19 nucleotides in length). In some embodiments, the antisense strand is 12 to 36 nucleotides in length. In some embodiments, the antisense strand is 17 to 36 nucleotides in length. In some embodiments, the antisense strand is 15 to 30 nucleotides in length. In some embodiments, the antisense strand is 19 to 29 nucleotides in length. In some embodiments, the antisense strand is 19 to 27 nucleotides in length. In some embodiments, the antisense strand is 21 to 27 nucleotides in length. In some embodiments, the antisense strand is 21 to 23 nucleotides in length.

In some embodiments, the duplex formed by the sense strand and antisense strand, also referred to as the first duplex or D1, can be 12 to 30 nucleotides (e.g., 12 to 30, 12 to 27, 15 to 25, 21 to 26, 18 to 30, or 19 to 30 nucleotides) in length. In some embodiments, the length of the duplex formed between a sense strand and antisense strand of an oligonucleotide is at least 12 nucleotides in length (e.g., at least 12, at least 15, at least 20, or at least 25 nucleotides in length). In some embodiments, the length of the duplex formed between a sense strand and antisense strand of an oligonucleotide is 12, 13, 14, 15, 16, 17, 18, 19, 20, 21, 22, 23, 24, 25, 26, 27, 28, 29, or 30 nucleotides in length. In some embodiments, the duplex formed by the sense strand and antisense strand has a length of 12-21 base pairs. In some embodiments, the duplex has a length of 13 to 20, 14 to 20, 15 to 20, 16 to 20, 17 to 20, 18 to 20, or 19 to 20 base pairs. In some embodiments, the duplex has a length of 12 to 23, 12 to 22, 12 to 21, 12 to 20, 12 to 19, 12 to 18, 12 to 17, 12 to 16, 12 to 15, 12 to 14, or 12 to 13 base pairs in length. In some embodiments, the duplex has a length of 12, 13, 14, 15, 16, 17, 18, 19, 20, 21, 22, 23, 24, 25, 26, 27, 28, 29, or 30 base pairs in length. In some embodiments, the duplex region is at least 19 base pairs in length. In some embodiments, duplex is 20 base pairs in length. In some embodiments, duplex is 21 base pairs in length.

In some embodiments, the oligonucleotide has a region of complementarity between the sense strand and the antisense strand. In some embodiments, the region of complementarity can be 12 to 30 nucleotides in length. In some embodiments, the region of complementarity between the sense and antisense strand is at least 12 nucleotides long (e.g., at least 12, at least 15, at least 20, or at least 25 nucleotides long). In some embodiments, the region of complementarity between the sense and antisense strand is 12, 13, 14, 15, 16, 17, 18, 19, 20, 21, 22, 23, 24, 25, 26, 27, 28, 29, or 30 nucleotides in length. In some embodiments, the region of complementarity between the sense strand and the antisense strand is 12 to 30, 12 to 27, 12 to 22, 15 to 25, 15 to 23, 21 to 26, 20 to 23, 18 to 30 or 19 to 30 nucleotides in length.

In some embodiments, the oligonucleotide having a sense strand and antisense strand has one or more (e.g., 1, 2, 3, 4, 5) mismatches between the sense strand and antisense strand. In some embodiments, the sense strand and the antisense strand can have up to 1, up to 2, up to 3, up to 4, up to 5, etc. mismatches provided that it maintains the ability to form a duplex under appropriate hybridization conditions. In some embodiments, if there is more than one mismatch between a sense and antisense strand, they may be positioned consecutively (e.g., 2, 3 or more in a row), or interspersed throughout the region of complementarity, provided that the oligonucleotide maintains the ability to form the duplex under appropriate hybridization conditions. In some embodiments, the first duplex (D1) contains one or more mismatches.

In some embodiments, the region of complementarity of an antisense sequence to the target RNA expressed in the glial cell is at least 12 nucleotides in length. In some embodiments, the region of complementarity of the antisense sequence to the target RNA expressed in the glial cell is at least 15 nucleotides in length. In some embodiments, the region of complementarity of the antisense sequence to the target RNA expressed in the glial cell is at least 19 nucleotides in length. In some embodiments, the region of complementarity of the sequence to the target RNA expressed in the glial cell is at least 21 nucleotides in length. In some embodiments, the region of complementarity is at least 12, at least 13, at least 14, at least 15, at least 16, at least 17, at least 18, at least 19, at least 20, or at least 21 nucleotides in length. In some embodiments, an oligonucleotide provided herein has a region of complementarity to the target RNA that is in the range of 12 to 30 (e.g., 12 to 30, 12 to 22, 15 to 25, 17 to 21, 18 to 27, 19 to 27, or 15 to 30) nucleotides in length. In some embodiments, an oligonucleotide provided herein has a region of complementarity to the target RNA that is 12, 13, 14, 15, 16, 17, 18, 19, 20, 21, 22, 23, 24, 25, 26, 27, 28, 29 or 30 nucleotides in length.

In some embodiments, an oligonucleotide disclosed herein comprises a region of complementarity (e.g., on an antisense strand of a double-stranded oligonucleotide) that is fully complementary to a sequence on the target RNA. In some embodiments, a region of complementarity of an oligonucleotide (e.g., on an antisense strand of a double-stranded oligonucleotide) is complementary to a contiguous sequence of nucleotides of a target RNA sequence that is in the range of 12 to 30 nucleotides (e.g., 12 to 25, 12 to 20, 12 to 18, 12 to 16, 12 to 14, 14 to 20, 14 to 18, 14 to 16, 16 to 20, 16 to 18, or 18 to 30) in length. In some embodiments, a region of complementarity of an oligonucleotide (e.g., on an antisense strand of a double-stranded oligonucleotide) is complementary to a contiguous sequence of nucleotides of a target RNA sequence that is 12, 13, 14, 15, 16, 17, 18, 19, 20, 21, 22 or 23 contiguous nucleotides in length.

In some embodiments, a region of complementarity to a target RNA sequence may have one or more mismatches compared with a corresponding sequence of the target RNA. In some embodiments, a region of complementarity for an oligonucleotide can have up to 1, up to 2, up to 3, up to 4, up to 5, etc. mismatches provided that it maintains the ability to form complementary base pairs with the target RNA sequence under appropriate hybridization conditions. Alternatively, in some embodiments, a region of complementarity on an oligonucleotide can have no more than 1, no more than 2, no more than 3, no more than 4, or no more than 5 mismatches provided that it maintains the ability to form complementary base pairs with target RNA sequence under appropriate hybridization conditions. In some embodiments, if there are more than one mismatches in a region of complementarity, they may be positioned consecutively (e.g., 2, 3, 4, or more in a row), or interspersed throughout the region of complementarity provided that the oligonucleotide maintains the ability to form complementary base pairs with the target RNA sequence under appropriate hybridization conditions. In some embodiments, the antisense strand has a nucleotide mismatch with the sequence of the target RNA to enhance discrimination between RNAs expressed from alleles of a gene with polymorphic sequences or to enhance activity in reducing expression of the target RNA.

In some embodiments, the oligonucleotide further comprises a stem-loop sequence or structure, wherein the stem loop sequence or structure comprises sequence regions S1-L-S2, wherein S1 is complementary to S2 and are capable of forming a second duplex (D2), and wherein L is a loop formed when S1 and S2 form the second duplex (D2). In some embodiments, the sense strand comprises at its 5′-end the stem loop sequence or hairpin. In some embodiments, the antisense strand comprises at its 3′ end a stem-loop sequence. Preferably, in some embodiments, the sense strand comprises at its 3′-end a stem loop sequence. In some embodiments, the second duplex D2 can be of various lengths. In some embodiments, the second duplex/D2 has a length of 1-6 base pairs. In some embodiments, the second duplex/D2 has a length of 2 to 6, 3 to 6, 4 to 6, 5 to 6, 1 to 5, 2 to 5, 3 to 5, or 4 to 5 base pairs. In some embodiments, the second duplex/D2 has a length of 1, 2, 3, 4, 5, or 6 base pairs. In some embodiments, the S1 sequence includes a sequence of the target RNA and the S2 sequence antisense sequence of the target RNA, wherein S1 and S2 form a duplex. In some embodiments, the S1 and S2 sequence is an artificial sequence unrelated to the target RNA sequence. In some embodiments, the second duplex is fully complementary. In some embodiments, the second duplex can contain one or more mismatches. In some embodiments, the sequence of S1 and S2 are chosen to stabilize formation of the loop L. An exemplary S1 sequence is GCAGCC and its corresponding S2 sequence GGCUGC.

In some embodiments, the loop L in the stem-loop sequence can be up to 30 nucleotides in length. In some embodiments, the loop L is up to 10, 15, 20 or 25 nucleotides in length. In some embodiments, the loop L is 3 to 6 nucleotides, 3 to 6 nucleotides, 3 to 5 nucleotides, or 3 to 4 nucleotides in length. In some embodiments, L is a tetraloop, pentaloop, or a triloop, as described herein. In some embodiments, the tetraloop is 4 nucleotides in length. In some embodiments, sequence of L is chosen to stabilize the loop structure formed when S1 and S2 form the second duplex. In some embodiments, L is comprised of ribonucleotides, deoxyribonucleotides, or mixtures thereof. In some embodiments, one or more of the nucleotides of L is modified. As further described below, in some embodiments, L is a sequence set forth as UNCG, GAAA, CUUG, d(GNNA), d(GNRA) d(GNAB), d(CNNG), and d(TNCG), where N represents any nucleotide. In some embodiments, wherein N is any one of U, A, C, G and R is G or A. In some embodiments, L is a sequence set forth as GAAA.

In some embodiments, the oligonucleotide has one or more overhanging nucleotides. In some embodiments, the overhanging nucleotide can be on the sense strand or the antisense strand, or the sense strand and the antisense strand. In some embodiments, the oligonucleotide comprises a 3′-overhang of one or more nucleotides in length. In some embodiments, a 3′-overhang of one or more nucleotides in length is present on the antisense strand, the sense strand, or the antisense strand and the sense strand. In some embodiments, the oligonucleotide comprises a 5′-overhang sequence of one or more nucleotides in length. In some embodiments, a 5′-overhang of one or more nucleotides in length is present on the antisense strand, the sense strand, or the antisense strand and the sense strand. In some embodiments, an oligonucleotide includes at least 1, at least 2, at least 3, at least 4, at least 5, at least 6, at least 7, at least 8, or more single-stranded nucleotides at its 3′-terminus. In some embodiments, an oligonucleotide includes 2, 3, 4, 5, 6, 7, 8, or more single-stranded nucleotides at its 3′-terminus. In some embodiments, an oligonucleotide includes at least 1, at least 2, at least 3, at least 4, at least 5, at least 6, at least 7, at least 8, or more single-stranded nucleotides at its 5′-terminus. In some embodiments, an oligonucleotide includes 2, 3, 4, 5, 6, 7, 8, or more single-stranded nucleotides at its 5′-terminus. In some embodiments, the sense strand comprises a 5′-overhang of one or more nucleotides in length (e.g., 2 nucleotide overhang at the 5′ end) and a 3′ overhang of one or more nucleotides in length (e.g., 2 nucleotide overhang at the 3′ end).

In some embodiments, an oligonucleotide comprises a 3′-overhang sequence of 1 or 2 nucleotides in length, in which the 3′-overhang sequence is present on the sense strand, antisense strand, or both sense and antisense strands. In some embodiments, the oligonucleotide has an antisense strand of 23 nucleotides and a sense strand of 21 nucleotides, in which the 3′-end of the sense strand and 5′-end of guide antisense strand form a blunt end, and where the antisense strand has a 2 nucleotide 3′ overhang. In some embodiments, the oligonucleotide has an antisense strand of 22 nucleotides and a sense strand of 20 nucleotides, in which the 3′-end of the sense strand and 5′-end of the antisense strand form a blunt end and where the antisense strand has a 2 nucleotide 3′ overhang.

In some embodiments, an oligonucleotide provided herein comprising a sense strand and an antisense strand has an asymmetric structure. In some embodiments, an oligonucleotide has an asymmetric structure, with a sense strand having a length of 36 nucleotides, and an antisense strand having a length of 22 nucleotides with 2 nucleotide 3′-overhang at its 3′-terminus. In some embodiments, an oligonucleotide has an asymmetric structure, with a sense strand having a length of 37 nucleotides, and an antisense strand having a length of 23 nucleotides with a 2-nucleotide overhang at its 3′-terminus.

In some embodiments, the sense strand and an antisense strand are separate oligonucleotides. In other words, the sense strand and the antisense strand are not covalently linked. In some embodiments, the sense strand and the antisense strand is a double stranded oligonucleotide containing a gap or nick between the sense strand and the antisense strand when in double stranded form. In some embodiments, the oligonucleotide comprises a sense strand and an antisense strand, wherein the sense strand further comprises a stem-loop sequence having a structure S1-L-S2, wherein the nick or gap is in the S2 region of the sense strand.

In some embodiments, the oligonucleotide is a single oligonucleotide, wherein the sense strand and the antisense strand are covalently linked to form a single oligonucleotide. In some embodiments, the oligonucleotide comprises a sense strand and an antisense strand, wherein the sense strand further comprises a stem-loop sequence having regions S1-L-S2, and wherein the antisense strand is covalently linked to the sense strand, e.g., the antisense strand is linked to S2 region of the sense strand by an internucleotide linkage.

In some embodiments, the oligonucleotide comprises a means for increasing resistance of the oligonucleotide to a phosphatase and/or nuclease, increasing hybridization efficiency, and/or enhancing in vivo stability. In some embodiments, the sense strand comprises a means for increasing resistance of the sense strand to a phosphatase and/or nuclease, increasing hybridization efficiency, and/or enhancing in vivo stability. In some embodiments, the antisense strand comprises a means for increasing resistance of the antisense strand to a phosphatase and/or nuclease, increasing hybridization efficiency, and/or enhancing in vivo stability. In some embodiments, both the sense strand and the antisense comprise a means for a phosphatase and/or nuclease, increasing hybridization efficiency, and/or enhancing in vivo stability. In some embodiments, where the oligonucleotide includes a stem-loop sequence, the stem-loop sequence comprises a means for increasing resistance to a phosphatase and/or nuclease, increasing hybridization efficiency, and/or enhancing in vivo stability. In some embodiments, the means for increasing resistance of an oligonucleotide to a phosphatase and/or nuclease, increasing hybridization efficiency, and/or enhancing in vivo stability includes, among others, modifications to the sugar residues, 5′-terminal phosphates, internucleoside linkages, and nucleobase, as further described herein.

In some embodiments, at least one of the nucleotides of the oligonucleotide are modified. In some embodiments, one or more of the nucleotides of the oligonucleotide are modified. In some embodiments, substantially all (e.g., 90% or greater) or all the nucleotides of an oligonucleotide are modified. In some embodiments, at least one nucleotide of the sense strand is modified. In some embodiments, at least one nucleotide of the antisense strand is modified. In some embodiments, at least 5%, 10%, 15%, 20%, 30%, 40%, 50%, 60%, 70%, 80%, 90%, or more of the nucleotides of the sense strand are modified. In some embodiments, all the nucleotides of the sense strand are modified. In some embodiments, at least 5%, 10%, 15%, 20%, 30%, 40%, 50%, 60%, 70%, 80%, 90%, or more of the nucleotides of the antisense strand are modified. In some embodiments, all the nucleotides of the antisense strand are modified. In some embodiments, at least the 5′-terminal nucleotide of the sense strand is modified. In some embodiments, at least the 3′-terminal nucleotide of the sense strand is modified. In some embodiments, at least the 5′-terminal nucleotide of the antisense strand is modified. In some embodiments, at least the 3′-terminal nucleotide of the antisense strand is modified.

In some embodiments, the 5′-terminal phosphate of the oligonucleotide is modified, e.g., with a phosphate analog such as phosphorothioate or 4′-phosphonate analog, for example to limit action of phosphatase and other enzymes. In some embodiments, the 5′-terminal phosphate of the sense strand is modified, e.g., with a phosphate analog such as phosphorothioate or 4′-phosphonate analog. In some embodiments, the 5′-terminal phosphate of the antisense strand is modified, e.g., with a phosphate analog such as phosphorothioate or 4′-phosphonate analog. In some embodiments, the 4′-carbon of the sugar of the 5′-nucleotide of the antisense strand comprises a phosphate analog. In some embodiments, the phosphate analog is oxymethylphosphonate, vinylphosphonate, or malonyphosphonate.

In some embodiments, the oligonucleotide comprises at least one modified internucleotide linkage, as further described herein. In some embodiments, at least one modified internucleotide linkage is a phosphorothioate linkage. In some embodiments, the modified internucleotide linkage is present on the oligonucleotide in defined regions or in a pattern. In some embodiments, the modified internucleotide linkage is within 1 to 4 nucleotides at the 5′-end of the sense strand, and/or within 1 to 4 nucleotides at the 3′-end of the sense strand. In some embodiments, the modified internucleotide linkage is within 1 to 4 nucleotides at the 5′-end of the antisense strand, and/or within 1 to 4 nucleotides at the 3′-end of the antisense strand.

In some embodiments, the internucleotide linkage between nucleotides at positions 1 and 2 of the sense strand (at the 5′-terminal end) is a modified internucleotide linkage, e.g., phosphorothioate. In some embodiments, the internucleotide linkage between nucleotides at positions 1 and 2 and at positions 2 and 3 and optionally at positions 3 and 4 of the antisense strand (at the 5′-terminal end) are a modified internucleotide linkage, e.g., phosphorothioate. In some embodiments, the internucleotide linkages between the last 2, preferably last 3 nucleotides, at the 3′-end of the antisense strand are a modified internucleotide linkage, e.g., phosphorothioate. For example, for an antisense strand of 22 nucleotides in length, the internucleotide linkages between nucleotides at positions 20 and 21 and at positions 21 and 22 have a modified internucleotide linkage, e.g., phosphorothioate.

In some embodiments, the modified nucleotide comprises a modification of the sugar moiety, for example a 2′-modification. In some embodiments, the 2′-modification is a 2′-fluoro or 2′-O-methyl. In some embodiments, at least the 5′-terminal nucleotide of the oligonucleotide is modified with a 2′-fluoro or 2′-O-methyl. In some embodiments, at least the 3′-terminal nucleotide of the oligonucleotide is modified with a 2′-fluoro or 2′-O-methyl. In some embodiments, at least the 5′-terminal nucleotide of the sense strand is modified with a 2′-fluoro or 2′-O-methyl. In some embodiments, at least the 3′-terminal nucleotide of the sense strand is modified with a 2′-fluoro or 2′-O-methyl. In some embodiments, at least the 5′-terminal nucleotide of the antisense strand is modified with a 2′-fluoro or 2′-O-methyl. In some embodiments, at least the 3′-terminal nucleotide of the antisense strand is modified with a 2′-fluoro or 2′-O-methyl. In some embodiments, at least 5%, 10%, 15%, 20%, 30%, 40%, 50%, 60%, 70%, 80%, 90%, or more of the nucleotides of the sense strand has a 2′ modification of the sugar moiety. In some embodiments, all of the nucleotides of the sense strand have a 2′ modification of the sugar moiety. In some embodiments, at least 5%, 10%, 15%, 20%, 30%, 40%, 50%, 60%, 70%, 80%, 90%, or more of the nucleotides of the antisense strand has a 2′ modification of the sugar moiety. In some embodiments, all of the nucleotides of the antisense strand have a 2′ modification of the sugar moiety. Other 2′-modifications of the sugar moiety, for example in place of the 2′-O-methyl, are described in more detail below.

In some embodiments, less than 50% to about 10% of the nucleotides of the oligonucleotide have a sugar moiety modified with a 2′-F. In some embodiments, less than 50% to about 10% of the nucleotides of the sense strand have a sugar moiety modified with a 2′-F. In some embodiments, less than 50% to about 10% of the nucleotides of the antisense strand have a sugar moiety modified with a 2′-F. In the foregoing embodiments, the remaining nucleotides of the oligonucleotide have a sugar moiety modified with a 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O[2-(methylamino)-2-oxoethyl] (2′-O-NMA), and 2′-deoxy-2′-fluoro-β-d-arabinonucleic acid (2′-FANA), preferably 2-O-methyl.

In some embodiments, the sense strand is 19 to 21 nucleotides in length and has one or more nucleotides up to 4 nucleotides at nucleotide positions 7 to 11, preferably one or more nucleotides at nucleotide positions 9, 10, 11, with a sugar moiety modified with a 2′-F. In some embodiments, the sense strand has the nucleotides at nucleotide positions 9, 10, 11 with a sugar moiety modified with a 2′-F. In the foregoing embodiments, the remaining nucleotides of the sense stand have a sugar moiety modified with a 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA), preferably 2-O-methyl.

In some embodiments, the antisense strand is 21 to 23 nucleotides in length and has one or more nucleotides, up to 6, up to 5, up to 4 or up to 3 of the nucleotides at nucleotide positions 1, 2, 3, 5, 6, 7, 10, 14, and 16, preferably nucleotide positions 2, 5, 6, 14, and 16, with a sugar moiety modified with a 2′-F. In some embodiments, the antisense strand has at least the nucleotide at nucleotide positions 5 or 14, or both nucleotide positions 5 and 14, with a sugar moiety modified with a 2′-F. In some embodiments, the antisense strand has nucleotides at nucleotide positions 2, 5 and 14, and optionally up to 3 nucleotides at nucleotide positions 1, 3, 6, 7, 10, and 16 with a sugar moiety modified with a 2′-F. In the foregoing embodiments, the remaining nucleotides of the antisense strand have a sugar moiety modified with a 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), and 2′-deoxy-2′-fluoro-β-d-arabinonucleic acid (2′-FANA), preferably 2-O-methyl.

In some embodiments, the oligonucleotide comprises a sense strand and strand, and further comprises a stem-loop sequence S1-L-S2, wherein the L is a tetraloop or triloop. In some embodiments, all of the nucleotides of S1 and S2 region have a sugar moiety modified with a 2′-O-propargyl, 2′-O-propylamin, 2′-amino, 2′-ethyl, 2′-aminoethyl (EA), 2′-O-methyl (2′-OMe), 2′-O-methoxy ethyl (2′-M0E), 2′-O[2-(methylamino)-2-oxoethyl] (2′-O-NMA), and 2′-deoxy-2′-fluoro-β-d-arabinonucleic acid (2′-FANA), preferably 2-O-methyl. In some embodiments, the 5′-terminal nucleotide of the L sequence has a modification of the sugar moiety with a 2′-O-methyl, and the remaining nucleotides of L have a targeting ligand, as further described below. In some embodiments, all the nucleotides of the L sequence have a targeting ligand, e.g., GalNAc.

In some embodiments, the oligonucleotide for selective delivery or selective reduction in levels of an RNA and/or protein encoded by the RNA expressed in a glial cell is optionally modified with one or more targeting ligands or moieties. In some embodiments, the oligonucleotide for selective delivery or selective reduction in levels of an RNA and/or protein encoded by the RNA expressed in a glial cell further comprises one or more targeting ligands or moieties. In some embodiments, the targeting ligand or moiety is selected from a carbohydrate, amino sugar, cholesterol, peptide, polypeptide, protein or part of a protein (e.g., an antibody or antibody fragment) or lipid. In some embodiments, the targeting ligand or moiety is one or more GalNAc residues or moieties.

In some embodiments, one or more nucleotides of an oligonucleotide are each conjugated to a separate targeting ligand. In some embodiments, 1, 2, 3, 4, 5 or 6 nucleotides of an oligonucleotide are each conjugated to a targeting ligand. In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., the ligand is conjugated to a 2 to 4 nucleotide overhang or extension on the 5′ or 3′ end of the sense or antisense strand). For example, an oligonucleotide may comprise a stem-loop sequence at either the 5′ or 3′ end of the sense strand and 1, 2, 3 or 4 nucleotides of the loop of the stem may be individually conjugated to a targeting ligand.

In some embodiments, an oligonucleotide is not conjugated to GalNAc. In certain embodiments, an oligonucleotide is conjugated directly or indirectly to a monovalent GalNAc. In some embodiments, the oligonucleotide is conjugated directly or indirectly to more than one monovalent GalNAc. In some embodiments, the oligonucleotide is conjugated directly or indirectly to 2, 3, or 4 monovalent GalNAc moieties, and is typically conjugated to 3 or 4 monovalent GalNAc moieties. In some embodiments, an oligonucleotide of the instant disclosure is conjugated to a one or more bivalent GalNAc, trivalent GalNAc, or tetravalent GalNAc moieties.

In some embodiments, one or more nucleotides of an oligonucleotide are each conjugated to a GalNAc moiety. In some embodiments, 1, 2, 3, 4, 5 or 6 nucleotides of a tetraloop of an oligonucleotide are conjugated to a GalNAc moiety. In some embodiments, 2 to 4 nucleotides of tetraloop are each conjugated to a separate GalNAc. In some embodiments, 1 to 3 nucleotides of triloop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand). In some embodiments, GalNAc moieties are conjugated to a nucleotide of the sense strand. In an exemplary embodiment, four GalNAc moieties can be conjugated to nucleotides in the tetraloop of the sense strand where each GalNAc moiety is conjugated to one nucleotide.

In some embodiments, an oligonucleotide herein comprises a monovalent GalNAc attached to a guanidine nucleotide, referred to as [ademG-GalNAc] or 2′-aminodiethoxymethanol-Guanidine-GalNAc, as depicted below:

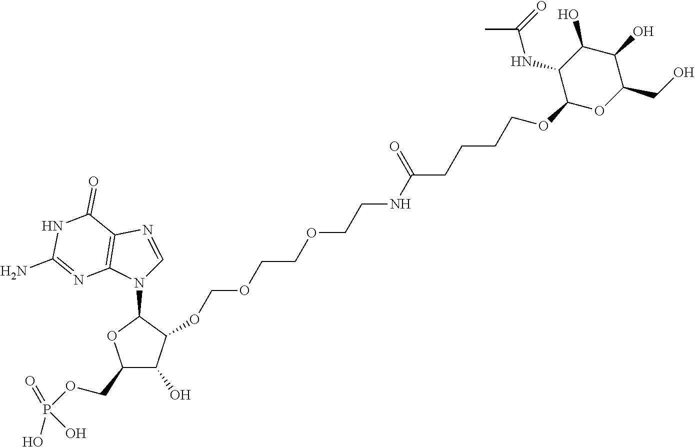 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, an oligonucleotide comprises a monovalent GalNAc attached to an adenine nucleotide, referred to as [ademA-GalNAc] or 2′-aminodiethoxymethanol-Adenine-GalNAc, as depicted below.

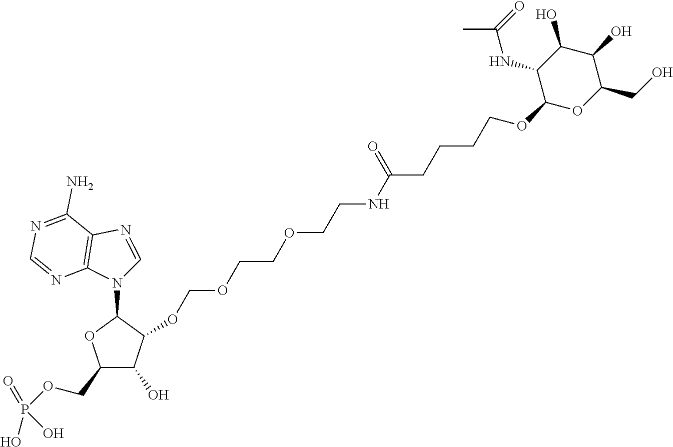 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(N)ncnc32)O[C@H](CO)[C@H](O)[C@@H]1O]

In some embodiments, an exemplary modification is shown below for a loop comprising from 5′ to 3′ the nucleotide sequence GAAA (Z=linker, X=heteroatom) stem-loop sequence. In the chemical formula,

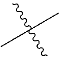

is used to describe an attachment point to the oligonucleotide strand:

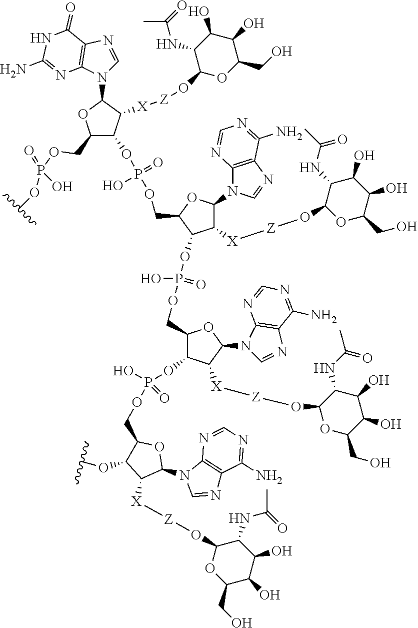

wherein:

Z represents a bond, click chemistry handle, or a linker of 1 to 20, inclusive, consecutive, covalently bonded atoms in length, selected from the group consisting of substituted and unsubstituted alkylene, substituted and unsubstituted alkenylene, substituted and unsubstituted alkynylene, substituted and unsubstituted heteroalkylene, substituted and unsubstituted heteroalkenylene, substituted and unsubstituted heteroalkynylene, and combinations thereof; and X is O, S, or N.

In some embodiments, Z is an acetal linker. In some embodiments, X is O.

In some embodiments, the -AAA- sequence of the loop L on the sense strand comprises the structure:

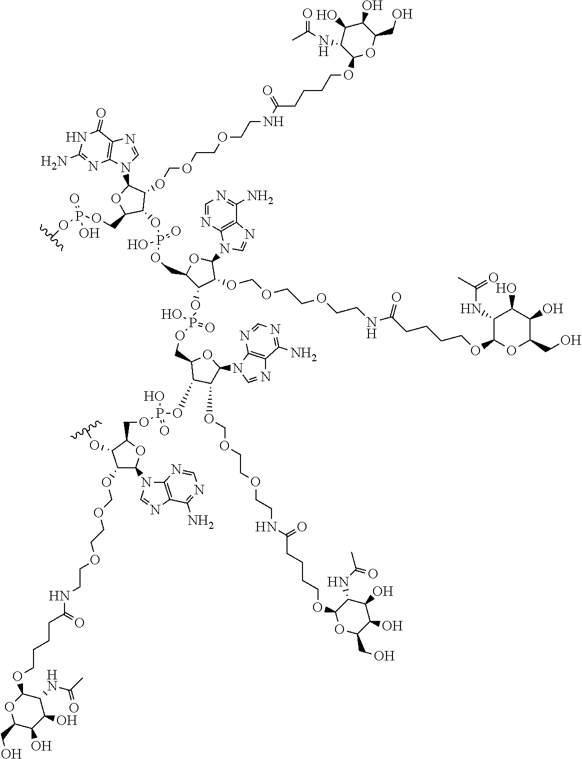 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

In some embodiments, loop L comprises a sequence set forth as GAAA. In some embodiments, each of the A in GAAA sequence is conjugated to a GalNAc moiety. In some embodiments, the G in the GAAA sequence comprises a 2′-O-methyl modification. In some embodiments, the G in the GAAA sequence comprises a 2′-OH. In some embodiments, each of the nucleotides in the GAAA sequence comprises a 2′-O-methyl modification. In some embodiments, each of the A in the GAAA sequence comprises a 2′-OH and the G in the GAAA sequence comprises a 2′-O-methyl modification. In some embodiments, each of the A in the GAAA sequence comprises a 2′-O-methoxyethyl modification and the G in the GAAA sequence comprises a 2′-O-methyl modification. In some embodiments, each of the A in the GAAA sequence comprises a 2′-adem modification and the G in the GAAA sequence comprises a 2′-O-methyl modification.

In some embodiments, a non-limiting example of an oligonucleotide for selective delivery of an interfering oligonucleotide or for selective reduction in levels of a target RNA and/or protein encoded by the RNA expressed in glial cells has the following structure (shown 5′→3′ from the sense strand to the antisense strand):

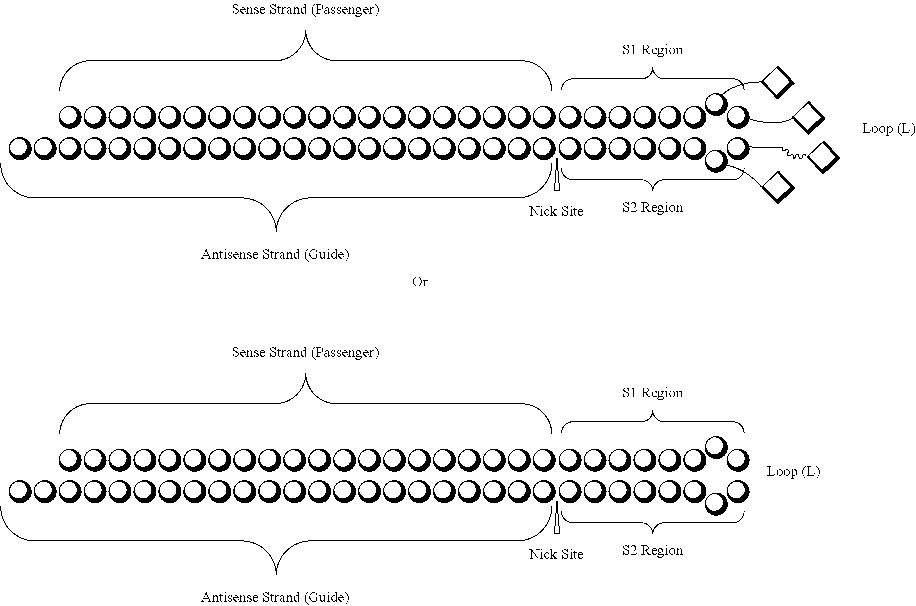

wherein each of the circles represent a nucleotide connected via internucleotide linkages, and the loop (L), shown as a tetraloop in the illustrated structure, has nucleotides conjugated to GalNAc residues (represented by diamond shapes). The numbering of the sense strands begins at the 5′-end of the sense strand, while the numbering of the antisense strand begins at the nucleotide residue following the nick site.

In some embodiments, the oligonucleotide is a single continuous oligonucleotide (i.e., no nick or gap is present between sense strand and antisense strand) and can be present in single stranded or double stranded form. In some embodiments, the oligonucleotide comprises separate sense strand and antisense strand, for example, when a nick or gap is present between the sense strand and the antisense strand, as shown in the illustrated structure, or where the sense strand and the antisense strand form a duplex with a blunt end between the 3′ terminus of the sense strand and the 5′ terminus of the antisense strand.

In some embodiments, the oligonucleotide comprises a sense strand and antisense strand that are each in the range of 17 to 36 nucleotides in length. In some embodiments, oligonucleotides have a tetraloop structure within a 3′ extension of their sense strand, and 2 nucleotide overhang at the 3′ end of the antisense strand. In some embodiments, the 2 nucleotide 3′ overhang is GG. Generally, in some embodiments, one or both of the two terminal GG nucleotides of the antisense strand is or are not complementary to the target RNA sequence.

In some embodiments, the oligonucleotides comprise a sense strand and antisense strand that are each in the range of 20 to 23 nucleotides in length. In some embodiments, a 3′ overhang is provided on the sense, antisense, or both sense and antisense strands that is 1 or 2 nucleotides in length. In some embodiments, an oligonucleotide has an antisense strand of 23 nucleotides and a sense strand of 21 nucleotides, in which the 3′-end of the sense strand and 5′-end of the antisense strand form a blunt end and where the guide strand has a two nucleotide 3′ overhang. In some embodiments, an oligonucleotide has an antisense strand of 22 nucleotides and a sense strand of 20 nucleotides, in which the 3′-end of the sense strand and 5′-end of the antisense strand form a blunt end and where the antisense strand has a two nucleotide 3′ overhang. In some embodiments, a 3′ overhang is provided on the antisense strand that is 9 nucleotides in length. For example, an oligonucleotide may have an antisense (guide) strand of 22 nucleotides and a passenger strand of 29 nucleotides, wherein the sense (passenger) strand forms tetraloop structure at its terminal 3′ end and the antisense (guide) strand has a 9 nucleotide 3′ overhang (herein termed “N-9”).

It is to be understood that the oligonucleotide of the illustrated structure can have variations in the length of the sense strand, antisense strand, the S1 and S2 regions, loop (L), and nucleotide overhang, and variations in optional targeting moieties, position of nick site, position of nucleotide overhang, type and degree of modification to the nucleotides of the oligonucleotide (including sense strand and/or antisense strand), and degree of complementarity of the sense strand and antisense strand and the S1 and S2 regions, as further described in detail herein. Each of the embodiments, variations and modifications described herein and any combination of such embodiments, variations and modifications apply to the exemplary oligonucleotide structure described above.

### b. Glial Cell Target RNA Sequences

In various embodiments, the antisense strand has a region of complementary to a target RNA of interest expressed in a glial cell. In some embodiments, the RNA of interest comprises a mRNA encoding a protein of interest. In some embodiments, the glial cell is present in the central nervous system or peripheral nervous system of a subject. In some embodiments, the glial cell is an astrocyte, oligodendrocyte, ependymal cell, microglial cell, Schwann cell, satellite cell, or enteric glial cell. In some embodiments, the antisense strand has a region of complementarity to an RNA expressed in an astrocyte, oligodendrocyte, ependymal cell, microglial cell, Schwann cell, satellite cell, or enteric glial cell.

In some embodiments, the target RNA of interest is an RNA and/or a corresponding encoded protein associated with a disease or disorder related to dysfunction of glial cells. In some embodiments, the target RNA and/or the associated disease or disorder include, by way of example and not limitation, expression of: glial fibrillary acidic protein (GFAP) gene (e.g., reference mRNA sequences: NM_001131019.3; NM_001242376.2; NM_001363846.1; and NM_002055.5) for Alexander Disease (AxD); prosaposin (PSAP) gene (e.g., reference mRNA sequence: NM_001042465.3; NM_001042466.3; and NM_002778.4) for Metachromatic Leukodystrophy; peripheral myelin protein 22 (PMP22) gene (e.g., reference mRNA sequence: NM_000304.4; NM_001281455.1; NM_001281456.2; NM_001330143.2; and NM_153321.3) for Charcot-Marie-Tooth disease; lamin B1 protein (LMNB1) gene (e.g., reference mRNA sequence: NM_001198557.2; and NM_005573.4) for Adult-Onset Leukodystrophy; amyloid precursor protein (APP) gene (e.g., reference mRNA sequences: NM_000484.4; NM_001136016.3; NM_001136129.3; NM_001136130.3; and NM_001136131.2), and microtubule-associated protein tau (MAPT) gene (e.g., reference mRNA sequence: NM_001123066.3; NM_001123067.3; NM_001203251.2; NM_001203252.1; and NM_005910.5) for Alzheimer's Disease; superoxide dismutase 1 (SOD1) gene (e.g., reference mRNA sequence NM_000454.4), chromosome 9 open reading frame 72 (C9orf72) gene (e.g., reference mRNA sequence: NM_001256054.2; NM_018325.5; and NM_145005.6), and Huntington (HTT) gene (e.g., reference mRNA sequence: NM_002111.8) for Huntington's Disease; α-synuclein (SNCA or ASYN) gene (e.g., reference mRNA sequence: NM_000345.4; NM_001146054.2; NM_001146055.2; and NM_007308.2) and dardarin/Leucine-rich repeat kinase 2 (LRRK2) gene (e.g., reference mRNA sequence: NM_198578.4) for Parkinson's Disease; adenosine kinase (ADK) gene (e.g., reference mRNA sequence: NM_001123.3; NM_001202449.1; NM_001202450.1; and NM_001369123.1; NM_001369124.1) for epilepsy; tumor necrosis factor alpha (TNFα) gene (e.g., reference mRNA sequence: NM_000594.4), and mitogen-activated protein kinase 7 (ERK5/MAPK7) gene (e.g., reference mRNA sequence: NM_002749.4; NM_139032.3; NM_139033.2; and NM_139034.3) for stroke; tumor necrosis factor alpha (TNFα) gene (e.g., reference mRNA sequence: NM_000594.4) and glial fibrillary acidic protein (GFAP) gene (e.g., reference mRNA sequences: NM_001131019.3; NM_001242376.2; NM_001363846.1; and NM_002055.5) for traumatic brain injury and axonal injury; IL-1 receptor type 2 (IL-1R2) gene (e.g., reference mRNA sequence; NM_001261419.2; NM_004633.4; and NM_173343.1) for autism; integrin subunit alpha 4 (CD49d) gene (e.g., reference mRNA sequence: NM_000885.6; and NM_001316312.1) for Multiple Sclerosis; Insulin-like growth factor 1 (IGF-1) gene (e.g., reference mRNA sequence: NM_000618.5; NM_001111283.3; NM_001111284.2; and NM_001111285.3), epidermal growth factor (EGF) gene (e.g., reference mRNA sequence: NM_001178130.3; NM_001178131.3; NM_001357021.2; and NM_001963.6), transforming growth factor beta (TGF-β) gene (e.g., reference mRNA sequence: NM_000660.7), and vascular endothelial growth factor (VEGF) gene (e.g., reference mRNA sequence: NM_001025366.3; NM_001025367.3; NM_001025368.3; NM_001025369.3; and NM_001025370.3) for glioblastoma and glial-cell cancer; superoxide dismutase 1 (SOD1) gene (e.g., reference mRNA sequence NM_000454.4), C9orf72 gene (e.g., reference mRNA sequence: NM_001256054.2; NM_018325.5; and NM_145005.6), and TAR-DNA-binding protein (TDP-43) gene (e.g., reference mRNA sequence: NM_007375.3) for amyotrophic lateral sclerosis (ALS); tumor necrosis factor alpha (TNFα) gene (e.g., reference mRNA sequence: NM_000594.4), and cluster of differentiation 38 (CD38) gene (e.g., reference mRNA sequence: NM_001775.4) for neuroinflammation; ataxin 2 (ATXN2) gene (e.g., reference mRNA sequence: NM_001310121.1; NM_001310123.1; and NM_002973.4), ataxin 3 (ATXN3) gene (e.g., reference mRNA sequence: NM_001127696.2; NM_001127697.2; NM_001164774.2; NM_001164776.2; and NM_001164777.2), and ataxin 7 (ATXN7) gene (e.g., reference mRNA sequence: NM_000333.3; NM_001128149.3; and NM_001177387.1) for spinocerebellar ataxias (1, 3, 5, 7, etc.); microtubule-associated protein tau (TAU/MAPT) gene (e.g., reference mRNA sequence: NM_001123066.3; NM_001123067.3; NM_001203251.2; NM_001203252.1; and NM_005910.5) for progressive supranuclear palsy; TAU/MAPT gene (e.g., reference mRNA sequence: NM_001123066.3; NM_001123067.3; NM_001203251.2; NM_001203252.1; and NM_005910.5) for primary age-related tauopathy (PART)/neurofibrillary tangle-predominant senile dementia; TAU/MAPT gene (e.g., reference mRNA sequence: NM_001123066.3; NM_001123067.3; NM_001203251.2; NM_001203252.1; and NM_005910.5) for frontotemporal dementia and Parkinsonism linked to chromosome 17 (FTDP-17); and Early growth response protein 2 (EGR2) gene (e.g., reference mRNA sequence: NM_000399.5; NM_001136177.3; NM_001136178.1; NM_001136179.3; and NM_001321037.2) for peripheral nerve demyelination.

In some embodiments, the target RNA is the processed form of the RNA. In some embodiments, the target RNA is the unprocessed form of the RNA. In some embodiments, the oligonucleotide, e.g., antisense strand, has a region of complementarity to the 5′-untranslated region of a target mRNA. In some embodiments, the oligonucleotide, e.g., antisense strand, has a region of complementarity to the coding region of a target mRNA. In some embodiments, the oligonucleotide, e.g., antisense strand, has a region of complementarity to an exon of a target mRNA. In some embodiments, the oligonucleotide, e.g., antisense strand, has a region of complementarity to the 3′-untranslated region of a target mRNA. Generally, the location and sequence of 5′-untranslated regions, 3′-untranslated regions, and exons of an mRNA for targeting with the antisense strand are provided in NCBI database for each of the NCBI reference mRNA sequence disclosed above. In some embodiments, the oligonucleotide, e.g., antisense strand, has a region of complementarity to sequences near or at splice junctions of the RNA, where the targeted sequence reduces or modulates expression of the target RNA.

In some embodiments, genes encoding mutated forms of a protein or genes whose overexpression in glial cells are associated with the disease or disorder are targeted for reduction in expression. In some embodiments, these include, by way of example and not limitation, GFAP, PMP22, LMNB1, APP, MAPT, SOD1, C9orf72, ATXN2, NG2, C9ORF72, ATXN3, ATXN7, HTT, SNCA/ASYN, ADK, TNFα, CD49d, TDP-43, and TAU/MAPT. In some embodiments, the antisense strand is fully complementary to the sequence containing the mutation in the target mRNA sequence and is effective in reducing expression of the RNA containing the mutation (see, e.g., Scholefield et al., PLOS One, 2009, 4 (9):e7232). In some embodiments, the target mRNA is an allele specific sequence of the RNA, such as the allele with the mutation (see, e.g., Bongianino et al., CIRCULATION RESEARCH, 2017, 121 (5):525-536). In some embodiments, the antisense strand is targeted to a single nucleotide polymorphism (SNP) associated with the disease associated mutation. The gene of interest could be in homozygous or heterozygous form.

Further description of oligonucleotide characteristics, variations, and modifications are described in the following sections. The oligonucleotides of the disclosure include each embodiment, variation, and modification described below, and any combination of such embodiments, variations, and modifications.

### c. Oligonucleotides Structures, Variations and Modifications

i. Antisense Strands

In some embodiments, an antisense strand of an oligonucleotide may be referred to as a “guide strand.” For example, if an antisense strand can engage with RNA-induced silencing complex (RISC) and bind to an Argonaute protein, or engage with or bind to one or more similar factors, and direct silencing of a target gene, it may be referred to as a guide strand. In some embodiments a sense strand complementary with a guide strand may be referred to as a “passenger strand.”

In some embodiments, an oligonucleotide comprises an antisense strand that is up to 50 nucleotides in length (e.g., up to 50, up to 45, up to 40, up to 35, up to 30, up to 27, up to 25, up to 21, up to 19, up to 17, or up to 12 nucleotides in length). In some embodiments, an oligonucleotide provided herein comprises an antisense strand is at least 12 nucleotides in length (e.g., at least 12, at least 15, at least 19, at least 21, at least 25, at least 27, at least 30, at least 35, or at least 38 nucleotides in length). In some embodiments, an antisense strand of an oligonucleotide disclosed herein is in the range of 12 to 50, 12 to 40, or 12 to 30 (e.g., 12 to 50, 12 to 40, 12 to 38, 12 to 36, 12 to 32, 12 to 30, 12 to 28, 12 to 22, 12 to 23, 15 to 21, 15 to 27, 17 to 21, 17 to 25, 19 to 27, or 19 to 30) nucleotides in length. In some embodiments, an antisense strand of any one of the oligonucleotides disclosed herein is 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In some embodiments, an antisense strand can comprise 19-23 nucleotides in length. In some embodiments, the antisense strand comprises 19-22 nucleotides in length. In some embodiments, the antisense strand comprises 23 nucleotides in length, 22 nucleotides in length, 21 nucleotides in length, 20 nucleotides in length, or 19 nucleotides in length.

In some embodiments, an antisense strand can comprise 20-22 nucleotides in length. In some embodiments, the antisense strand comprises 20-21 nucleotides in length or 21-22 nucleotides in length. In some embodiments, the antisense strand comprises 20 nucleotides in length, 21 nucleotides in length, or 22 nucleotides in length.

An oligonucleotide having an asymmetric structure as provided herein may include an antisense strand having any length of single-stranded nucleotides at its 3′-terminus (i.e., 3′-overhang). In some embodiments, the antisense strand includes at least 2 single-stranded nucleotides at its 3′-terminus. In some embodiments, the antisense strand includes at least 0, 1, 2, 3, at least 4, at least 5, at least 6 or more single-stranded nucleotides at its 3′-terminus. In some embodiments, the antisense strand includes 2 single-stranded nucleotides at its 3′-terminus. In some embodiments, the antisense strand includes 3 single-stranded nucleotides at its 3′-terminus. In some embodiments, the antisense strand includes 4 single-stranded nucleotides at its 3′-terminus. In some embodiments, the antisense strand includes 5 single-stranded nucleotides at its 3′-terminus. In some embodiments, the antisense strand includes 6 single-stranded nucleotides at its 3′-terminus.

ii. Sense Strands

Oligonucleotides provided herein, in some embodiments, comprise a sense strand. In some embodiments, an oligonucleotide may have a sense strand (or passenger strand) of up to 40 nucleotides in length (e.g., up to 40, up to 35, up to 30, up to 27, up to 25, up to 21, up to 19, up to 17, or up to 12 nucleotides in length).

In some embodiments, an oligonucleotide may have a sense strand of at least 12 nucleotides in length (e.g., at least 12, at least 15, at least 19, at least 21, at least 25, at least 27, at least 30, at least 32, at least 34, at least 36, or at least 38 nucleotides in length). In some embodiments, an oligonucleotide may have a sense strand in a range of 12 to 40 (e.g., 12 to 40, 12 to 36, 12 to 32, 12 to 28, 15 to 40, 15 to 36, 15 to 32, 15 to 28, 17 to 21, 17 to 25, 17 to 36, 19 to 27, 19 to 30, 20 to 40, 22 to 40, 25 to 40, or 32 to 40) nucleotides in length. In some embodiments, an oligonucleotide may have a sense strand of 12 nucleotides in length, 13 nucleotides in length, 14 nucleotides in length, 15 nucleotides in length, 16 nucleotides in length, 17 nucleotides in length, 18 nucleotides in length, 19 nucleotides in length, 20 nucleotides in length, 21 nucleotides in length, 22 nucleotides in length, 23 nucleotides in length, 24 nucleotides in length, 25 nucleotides in length, 26 nucleotides in length, 27 nucleotides in length, 28 nucleotides in length, 29 nucleotides in length, 30 nucleotides in length, 31 nucleotides in length, 32 nucleotides in length, 33 nucleotides in length, 34 nucleotides in length, 35 nucleotides in length, 36 nucleotides in length, 37 nucleotides in length, 38 nucleotides in length, 39 nucleotides in length, or 40 nucleotides in length.

In some embodiments, the sense strand further comprises at its 3′-end a stem-loop sequence (or structure) having regions S1-L-S2, wherein S1 region is complementary to S2 region and are capable of forming a second duplex (D2) or stem, and wherein L is a loop formed when S1 and S2 form D2 (stem). In some embodiments, D2 or stem formed between S1 and S2 is at least 1 (e.g., at least 2, at least 3, at least 4, at least 5, or at least 6) base pairs in length. In some embodiments, D2 or stem is a duplex of 2, 3, 4, S, 6, 7, 8, 9, 10, 11, 12, 13, or 14 nucleotides in length. In some embodiments, D2 formed between S1 region and S2 region is in the range of 1-6 base pairs in length (e.g., 1-5, 1-4, 1-3, 1-2, 2-6, 3-6, 4-6, or 5-6 base pairs in length).

In some embodiments, loop L formed between S1 and S2 is of up to 10 nucleotides in length (e.g., 3, 4, S, 6, 7, 8, 9, or 10 nucleotides in length). In some embodiments, the loop L comprises a tetraloop, pentaloop or a triloop (triL) that joins the S1 and S2 regions. In some embodiments, the tetraloop, the pentaloop or the triloop is at the 3′ terminus of the sense strand. In some embodiments, the tetraloop, the pentaloop or the triloop is at the 5′ terminus of the antisense strand rather than the sense strand.

In some embodiments, any number of nucleotides in the L, such as a triloop, pentaloop or a tetraloop, may be conjugated to a targeting ligand. In some embodiments, a triloop comprises 1 nucleotide that is conjugated to a ligand. In some embodiments, a triloop comprises 2 nucleotides that are conjugated to a ligand. In some embodiments, a triloop comprises 3 nucleotides that are conjugated to a ligand. In some embodiments, a triloop comprises 1-3 nucleotides that are conjugated to a ligand. In some embodiments, a triloop comprises 1-2 nucleotides that are conjugated to a ligand or 2-3 nucleotides that are conjugated to a ligand.

In some embodiments, a tetraloop comprises 1 nucleotide that is conjugated to a ligand. In some embodiments, a tetraloop comprises 2 nucleotides that are conjugated to a ligand. In some embodiments, a tetraloop comprises 3 nucleotides that are conjugated to a ligand. In some embodiments, a tetraloop comprises 4 nucleotides that are conjugated to a ligand. In some embodiments, a tetraloop comprises 1-4 nucleotides that are conjugated to a ligand. In some embodiments, a tetraloop comprises 1-3 nucleotides, 1-2 nucleotides, 2-4 nucleotides, or 3-4 nucleotides that are conjugated to a ligand.

In some embodiments, a tetraloop or a triloop may contain ribonucleotides, deoxyribonucleotides, modified nucleotides, and combinations thereof. Non-limiting examples of a RNA tetraloop include, but are not limited to, the UNCG family of tetraloops (e.g., UUCG), the GNRA family of tetraloops (e.g., GAAA), and the CUUG tetraloop. Non-limiting examples of, DNA tetraloops include, but are not limited to, the d(GNNA) family of tetraloops (e.g., d(GTTA)), the d(GNRA) family of tetraloops, the d(GNAB) family of tetraloops, the d(CNNG) family of tetraloops, and the d(TNCG) family of tetraloops (e.g., d(TTCG)).

iii. Duplex Length

In some embodiments, a duplex formed between a sense strand and antisense strand is at least 12 (e.g., at least 15, at least 16, at least 17, at least 18, at least 19, at least 20, or at least 21) nucleotides in length. In some embodiments, a duplex formed between a sense strand and antisense strand is in the range of 12-30 nucleotides in length (e.g., 12 to 30, 12 to 27, 12 to 22, 15 to 25, 18 to 22, 18 to 25, 18 to 27, 18 to 30, 19 to 30 or 21 to 30 nucleotides in length). In some embodiments, a duplex formed between a sense strand and antisense strand is 12, 13, 14, 15, 16, 17, 18, 19, 20, 21, 22, 23, 24, 25, 26, 27, 28, 29, or 30 nucleotides in length. In some embodiments a duplex formed between a sense strand and antisense strand does not span the entire length of the sense strand and/or antisense strand. In some embodiments, a duplex between a sense and antisense strand spans the entire length of either the sense or antisense strands. In some embodiments, a duplex between a sense and antisense strand spans the entire length of both the sense strand and the antisense strand.

iii. Oligonucleotide Ends

In some embodiments, an oligonucleotide provided herein comprises sense strand and antisense strand, wherein a 3′-overhang is present on either the sense strand or the antisense strand, or both the sense and antisense strand. In some embodiments, the oligonucleotides of the disclosure have one 5′-end that is thermodynamically less stable compared to the other 5′ end. In some embodiments, an asymmetric oligonucleotide is provided that includes a blunt end at the 3′-end of a sense strand and an overhang at the 3′-end of an antisense strand. In some embodiments, a 3′ overhang on an antisense strand is 1-8 nucleotides in length (e.g., 1, 2, 3. 4, 5, 6, 7 or 8 nucleotides in length).

Typically, an oligonucleotide for RNAi has a two-nucleotide overhang on the 3′-end of the antisense (guide) strand. However, other overhangs are possible. In some embodiments, an overhang is a 3′-overhang comprising a length of between 1 and 6 nucleotides. optionally 1 to 5, 1 to 4, 1 to 3, 1 to 2, 2 to 6, 2 to 5, 2 to 4, 2 to 3, 3 to 6, 3 to 5, 3 to 4, 4 to 6, 4 to 5, 5 to 6 nucleotides, or 1, 2, 3, 4, 5 or 6 nucleotides. However, in some embodiments, the overhang is a 5′-overhang comprising a length of between 1 and 6 nucleotides, optionally 1 to 5, 1 to 4, 1 to 3. 1 to 2, 2 to 6, 2 to 5, 2 to 4, 2 to 3, 3 to 6, 3 to 5. 3 to 4, 4 to 6, 4 to 5, 5 to 6 nucleotides, or 1, 2, 3, 4, 5 or 6 nucleotides.

In some embodiments, one or more (e.g., 2, 3, 4) terminal nucleotides of the 3′-end or 5′-end of a sense and/or antisense strand are modified. For example, in some embodiments, 1 or 2 terminal nucleotides of the 3′-end of an antisense strand are modified. In some embodiments, the last nucleotide at the 3′-end of an antisense strand is modified, e.g., comprises 2′-modification, e.g., a 2′-O-methoxyethyl. In some embodiments, the last one or two terminal nucleotides at the 3′-end of an antisense strand are complementary to the target. In some embodiments, last one or two nucleotides at the 3′-end of the antisense strand are not complementary to the target. In some embodiments, the 5′-end and/or the 3′-end of sense or antisense strand has an inverted cap nucleotide.

iv. Mismatches

In some embodiments, an oligonucleotide comprised of a sense strand and an antisense strand has one or more (e.g., I, 2, 3, 4, 5) mismatches between a sense and antisense strand. If there is more than one mismatch between a sense and antisense strand, they may be positioned consecutively (e.g., 2, 3 or more in a row), or interspersed throughout the region of complementarity. In some embodiments, the 3′-terminus of the sense strand contains one or more mismatches. In some embodiments, two mismatches are incorporated at the 3′-terminus of the sense strand. In some embodiments, base mismatches or destabilization of segments at the 3′-end of the sense strand of the oligonucleotide improved the potency of synthetic duplexes in RNAi, possibly through facilitating processing by Dicer.

v. Single Stranded Oligonucleotides

In some embodiments, an oligonucleotide for reducing expression of a target RNA is single-stranded. Such structures may include but are not limited to single-stranded RNAi oligonucleotides. (see, e.g., Matsui et al., MOLECULAR THERAPY, 2016, 24 (5), 946-955). In some embodiments, the single stranded RNAi is modified to enhance stability and effectiveness against the target RNA.

However, in some embodiments, oligonucleotides provided herein are antisense oligonucleotides (ASOs). An antisense oligonucleotide is a single-stranded oligonucleotide that has a nucleobase sequence which, when written in the 5′ to 3′ direction, comprises the reverse complement of a targeted segment of a particular nucleic acid and is suitably modified (e.g., as a gapmer) so as to induce RNaseH mediated cleavage of its target RNA in cells or (e.g., as a mixmer) and inhibit translation of the target mRNA in cells. Antisense oligonucleotides for use in the instant disclosure may be modified in any suitable manner known in the art including, for example, as shown in U.S. Pat. No. 9,567,587, which is incorporated by reference herein for its disclosure regarding modification of antisense oligonucleotides (including, e.g., length, sugar moieties of the nucleobase (pyrimidine, purine), and alterations of the heterocyclic portion of the nucleobase). Further, antisense molecules are used to reduce expression of specific target genes (see, e.g., Bennett et al., Pharmacology of Antisense Drugs, ANNUAL REVIEW OF PHARMACOLOGY AND TOXICOLOGY, 7:81-105).

iv. Oligonucleotide Modifications

Oligonucleotides can be modified in various ways to improve or control specificity, stability, delivery, bioavailability, resistance from nuclease degradation, immunogenicity, base-paring properties, RNA distribution and cellular uptake and other features relevant to therapeutic or research use (see, e.g., Bramsen et al., NUCLEIC ACIDS RES., 2009, 37:2867-2881; Bramsen and Kjems, Frontiers in Genetics, 2012, 3:1-22). Accordingly, in some embodiments, oligonucleotides of the present disclosure may include one or more suitable modifications. In some embodiments, a modified nucleotide has a modification in its base (or nucleobase), the sugar (e.g., ribose, deoxyribose), or the phosphate group, as further described below.

The number of modifications on an oligonucleotide and the positions of those nucleotide modifications may influence the properties of an oligonucleotide. For example, oligonucleotides may be delivered in vivo by conjugating them to or encompassing them in a lipid nanoparticle (LNP) or similar carrier. However, when an oligonucleotide is not protected by an LNP or similar carrier, it may be advantageous for at least some of its nucleotides to be modified. Accordingly, in certain embodiments of any of the oligonucleotides provided herein, all, or substantially all the nucleotides of an oligonucleotide are modified. In certain embodiments, more than half of the nucleotides are modified. In certain embodiments, less than half of the nucleotides are modified. Typically, with naked delivery, every sugar is modified at the 2′-position. These modifications may be reversible or irreversible. In some embodiments, an oligonucleotide as disclosed herein has a number and type of modified nucleotides sufficient to cause the desired characteristic (e.g., protection from enzymatic degradation, capacity to target a desired cell after in vivo administration, and/or thermodynamic stability).

### a. Sugar Modifications

In some embodiments, a modified sugar (also referred herein to a sugar analog) includes a modified deoxyribose or ribose moiety, e.g., in which one or more modifications occur at the 2′, 3′, 4′, and/or 5′ carbon position of the sugar. In some embodiments, a modified sugar may also include non-natural alternative carbon structures such as those present in locked nucleic acids (“LNA” or “bicyclic”) (see, e.g., Koshkin et al., TETRAHEDRON, 1998, 54:3607-3630), unlocked nucleic acids (“UNA” or “acyclic”) (see, e.g., Snead et al., MOLECULAR THERAPY—NUCLEIC ACIDS, 2013, 2:e103), and bridged nucleic acids (“BNA”) (see, e.g., Imanishi and Obika, THE ROYAL SOCIETY OFCHEMISTRY, CHEM. COMMUN., 2002, 1653-1659). Koshkin et al., Snead et al., and Imanishi and Obika are incorporated by reference herein for their disclosures relating to sugar modifications. The sugar modifications can increase resistance of the oligonucleotide to nucleases and/or enhance hybridization efficiency.

In some embodiments, a nucleotide modification of a sugar comprises a 2′-modification. In some embodiments, a 2′-modification includes, among others, 2′-O-propargyl, 2′-O-propylamin, 2′-amino, 2′-ethyl, 2′-aminoethyl (EA), 2′-O-methyl (2′-OMe), 2′-fluoro (2′-F), 2′-O-methoxyethyl (2′-MOE), 2′-O-[2-(methylamino)-2-oxoethyl] (2′-O-NMA), 2′-deoxy-2′-fluoro-β-d-arabinonucleic acid (2′-FANA), or combinations thereof. In some embodiments, the modification is 2′-fluoro, 2′-O-methyl, or 2′-O-methoxyethyl. In some embodiments, a modification of a sugar comprises a modification of the sugar ring, which may comprise modification of one or more carbons of the sugar ring. For example, a modification of a sugar of a nucleotide may comprise a 2′-oxygen of a sugar which is linked to a 1′-carbon or 4′-carbon of the sugar, or a 2′-oxygen which is linked to the 1′-carbon or 4′-carbon via an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′ position of the sugar.

In some embodiments, the oligonucleotide described herein comprises at least one nucleotide modified on the sugar moiety (e.g., at least 1, at least 5, at least 10, at least 15, at least 20, at least 25, at least 30, at least 35, at least 40, at least 45, at least 50, at least 55, at least 60, or more). In some embodiments, the sense strand of the oligonucleotide comprises at least one nucleotide modified on the sugar moiety (e.g., at least 1, at least 5, at least 10, at least 15, at least 20, at least 25, at least 30, at least 35, or more). In some embodiments, the antisense strand of the oligonucleotide comprises at least one nucleotide modified on the sugar moiety (e.g., at least 1, at least 5, at least 10, at least 15, at least 20, or more).

In some embodiments, all the nucleotides of the sense strand of the oligonucleotide have a modification of the sugar moiety. In some embodiments, all the nucleotides of the antisense strand of the oligonucleotide have a modification of the sugar moiety. In some embodiments, all the nucleotides of the oligonucleotide (i.e., both the sense strand and the antisense strand) have a modification of the sugar moiety. In some embodiments, the modification of the sugar moiety on the nucleotide comprises a 2′-modification (e.g., a 2′-fluoro, 2′-O-methyl, 2′-O-methoxyethyl, or 2′-deoxy-2′-fluoro-β-d-arabinonucleic acid). In some embodiments, the modification of the sugar moiety on the nucleotide comprises a 2′-modification with a 2′-fluoro or 2′-O-methyl.

In some embodiments, less than 50%, less than 40%, less than 35%, less than 30% to about 10%, about 12%, about 14%, about 18% or about 20% of the nucleotides of the sense strand have a sugar moiety modified with a 2′-F group. In some embodiments, less than 50%, less than 40%, less than 35%, less than 30% to about 10%, about 12%, about 14%, about 18% or about 20% of the nucleotides of the antisense strand have a sugar moiety modified with a 2′-F group. In some embodiments, a sense strand of 19 to 21 nucleotides in length has up to 5, preferably up to 4 nucleotides with modifications of the sugar moiety with a 2′-F group. In some embodiments, an antisense strand of 21 to 23 nucleotides in length has up to 6, preferably up to 5, more preferably up to 3 nucleotides with modifications of the sugar moiety with a 2′-F group. In some embodiments, an oligonucleotide having a sense strand of 19 to 21 nucleotides in length and an antisense strand of 21 to 23 nucleotides in length has up to a total of 4 to 12, 5 to 11, or 6 to 10 of the nucleotides with modification of the sugar moiety with a 2′-F. In some embodiments, the remaining nucleotides of the sense strand and antisense strand are modified on the sugar moiety with 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), 2′-deoxy-2′-fluoro-β-d-arabinonucleic acid (2′-FANA), or combinations thereof, preferably 2′-O-methyl.

In some embodiments, the oligonucleotide can have modifications to the sugar residue of nucleotides at defined positions of the oligonucleotide. In some embodiments, for an oligonucleotide comprising a sense stand and an antisense strand, the sense strand has one or more of nucleotides at nucleotide positions 7, 8, 9, 10, and 11, preferably one or more of nucleotides at nucleotide positions 9, 10 and 11, with the sugar moiety modified with a 2′-F. In some embodiments, the sense strand has 2, 3, 4 or all the nucleotides at nucleotide positions 7, 8, 9, 10, and 11 with modification of the sugar moiety with a 2′-F. As noted above, in some embodiments, the sense strand has up to 5, preferably up to 4 nucleotides with the sugar moiety modified with a 2′-F group.

In some embodiments, for each of the foregoing embodiments in which the sense strand has a nucleotide with the sugar moiety modified with a 2′-F, the remaining nucleotides of the sense strand has modification of the sugar moiety with 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), 2′-deoxy-2′-fluoro-β-d-arabinonucleic acid (2′-FANA), or combinations thereof, preferably 2′-O-methyl. In some embodiments, the sense strand has one or more of nucleotides at nucleotide positions 7, 8, 9, 10, and 11 with the sugar moiety modified with a 2′-F, and the remaining nucleotides of the sense strand have the sugar moiety modified with 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O[2-(methylamino)-2-oxoethyl] (2′-O-NMA), 2′-deoxy-2′-fluoro-β-d-arabinonucleic acid (2′-FANA), or combinations thereof, preferably 2′-O-methyl. In some embodiments, an oligonucleotide comprises a sense strand of 19 to 21 nucleotides in length, wherein sense strand has nucleotides at nucleotide positions 8, 9, 10, and 11, or preferably nucleotide positions 9, 10, and 11, with modification of the sugar moiety with a 2′-F, and each of the nucleotides at the remaining positions of the sense strand has modification of the sugar residue with a 2′-O-methyl.

In some embodiments, the antisense strand of the oligonucleotide has one or more of nucleotides at nucleotide positions 1, 2, 3, 5, 6, 7, 10, 14, and 16, preferably at one or more of nucleotide positions 2, 3, 5, 6, 7, 10 and 14, with the sugar moiety modified with a 2′-F. In some embodiments, the antisense strand has at least the nucleotide at nucleotide position 5 or 14 with the sugar moiety modified with a 2′-F. In some embodiments, the antisense strand has the nucleotides at nucleotide positions 5 and 14 with the sugar moiety modified with a 2′-F. In some embodiments, the antisense strand has the nucleotide at nucleotide position 5, and up to 5 of the nucleotides at nucleotide positions 1, 2, 3, 6, 7, 10, 14, and 16 with the sugar moiety modified with a 2′-F. In some embodiments, the antisense strand has a 2′-F modification at nucleotide position 14, and up to 5 of the nucleotides at nucleotide positions 1, 2, 3, 6, 7, 10, 14, and 16 with the sugar moiety modified with a 2′-F. In some embodiments, the antisense strand has the nucleotide at nucleotide positions 5 and 14, and up to 4 nucleotides at nucleotide positions 1, 2, 3, 6, 7, 10, and 16 with the sugar moiety modified with a 2′-F. In some embodiments, the antisense strand has the nucleotides at nucleotide positions 2, 5, and 14 and optionally up to 3 of the nucleotides at nucleotide positions 1, 3, 7, and 10 with the sugar moiety modified with a 2′-F. In some embodiments, the antisense strand has the nucleotide at each of the positions 2, 5, and 14 with the sugar moiety modified with a 2′-F. In some embodiments, the antisense strand has the nucleotide at each of the nucleotide positions 1, 2, 5, and 14 with the sugar moiety modified with a 2′-F. In some embodiments, the sugar moiety at each of the positions at positions 1, 2, 3, 5, 7, and 14 of the antisense strand is modified with the 2′-F. In yet another embodiment, the sugar moiety at each of the positions at positions 1, 2, 3, 5, 10, and 14 of the antisense strand is modified with the 2′-F. In another embodiment, the sugar moiety at each of the positions at positions 2, 3, 5, 7, 10, and 14 of the antisense strand is modified with the 2′-F. As noted above, in some embodiments, an antisense strand of 21 to 23 nucleotides in length has up to 6, preferably up to 5, more preferably up to 3 nucleotides with the sugar moiety modified with a 2′-F.

In some embodiments, for each of the foregoing embodiments in which the antisense strand has a nucleotide with the sugar moiety modified with a 2′-F, the remaining nucleotides of the antisense strand have the sugar moiety modified with 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), 2′-deoxy-2′-fluoro-β-d-arabinonucleic acid (2′-FANA), or combinations thereof, preferably 2′-O-methyl. In some embodiments, the antisense strand of the oligonucleotide has one or more of nucleotides at nucleotide positions 1, 2, 3, 5, 6, 7, 10, 14, and 16, preferably at one or more of nucleotide positions 2, 3, 5, 6, 7, 10 and 14, with the sugar moiety modified with a 2′-F, and the remaining nucleotides of the antisense strand have the sugar moiety modified with 2′-O-propargyl, 2′-O-propylamin, 2′-amino, 2′-ethyl, 2′-aminoethyl (EA), 2′-O-methyl (2′-OMe), 2′-O-methoxyethyl (2′-MOE), 2′-O-[2-(methylamino)-2-oxoethyl] (2′-O-NMA), 2′-deoxy-2′-fluoro-β-d-arabinonucleic acid (2′-FANA), or combinations thereof, preferably 2′-O-methyl. In some embodiments, the antisense strand has the nucleotides at nucleotide positions 2, 5, and 14, and optionally up to 3 of the nucleotides at nucleotide positions 1, 3, 6, 7, and 10 with modification of the sugar moiety with a 2′-F, and the remaining nucleotides of the antisense strand have modification of the sugar moiety with a 2′-O-methyl.

In some embodiments, for oligonucleotides having a stem-loop sequence of S1-L-S2, one or more nucleotides of the stem and/or loop have a modification of the sugar moiety. In some embodiments, the modification is exclusive of the modification with a targeting ligand. In some embodiments, the modification is 2′-O-propargyl, 2′-O-propylamin, 2′-amino, 2′-ethyl, 2′-aminoethyl (EA), 2′-O-methyl (2′-OMe), 2′-fluoro (2′-F), 2′-O-methoxyethyl (2′-MOE), 2′-O-[2-(methylamino)-2-oxoethyl] (2′-O-NMA), 2′-deoxy-2′-fluoro-β-d-arabinonucleic acid (2′-FANA), or combinations thereof. In some embodiments, the modification of the sugar moiety is 2′-fluoro, 2′-O-methyl, or 2′-O-methoxyethyl. In some embodiments, one or more of the nucleotides up to all of the nucleotides of S1 has a modification of the sugar moiety. In some embodiments, one or more of the nucleotides up to all of the nucleotide of S2 has a modification of the sugar moiety. In some embodiments, all of the nucleotides of S1 and S2 have a modification of the sugar moiety, preferably with a 2′-O-methyl. In some embodiments, one or more nucleotides of the loop L has a modification of the sugar moiety. In some embodiments, the 5′ nucleotide of the loop sequence has a modification of the sugar moiety. In some embodiments, for a tetraloop, pentaloop or triloop, the 5′ nucleotide of the loop sequence has a modification of the sugar moiety at the 2′ position, preferably with a 2′-O-methyl, and the remaining nucleotides of the loop are modified with targeting ligand.

### b. 5′ Terminal Phosphates

In some embodiments, 5′-terminal phosphate groups of oligonucleotides enhance the interaction with Argonaute 2. However, oligonucleotides comprising a 5′-phosphate group may be susceptible to degradation via phosphatases or other enzymes, which can limit oligonucleotide bioavailability in vivo. In some embodiments, oligonucleotides include analogs of 5′ phosphates that are resistant to such degradation. In some embodiments, a phosphate analog may be oxymethylphosphonate, vinylphosphonate, or malonylphosphonate. In certain embodiments, the 5′ end of an oligonucleotide strand is attached to chemical moiety that mimics the electrostatic and steric properties of a natural 5′-phosphate group (“phosphate mimic”) (see, e.g., Prakash et al., NUCLEIC ACIDS RES., 2015, 43 (6): 2993-3011, the contents of which relating to phosphate analogs are incorporated herein by reference). Many phosphate mimics have been developed that can be attached to the 5′ end (see, e.g., U.S. Pat. No. 8,927,513, the contents of which relating to phosphate analogs are incorporated herein by reference). Other modifications for the 5′ end of oligonucleotides are disclosed in e.g., International patent publication WO 2011/133871, the contents of which relating to phosphate analogs are incorporated herein by reference. In certain embodiments, a hydroxyl group is attached to the 5′ end of the oligonucleotide.

In some embodiments, an oligonucleotide has a phosphate analog at a 4′-carbon position of the sugar (referred to as a “4′-phosphate analog”). See, e.g., International Patent Application PCT/US2017/049909, filed on Sep. 1, 2017, U.S. Provisional Application Nos. 62/383,207, entitled 4′-Phosphate Analogs and Oligonucleotides Comprising the Same, filed on Sep. 2, 2016, and 62/393,401, filed on Sep. 12, 2016, entitled 4′-Phosphate Analogs and Oligonucleotides Comprising the Same, the contents of each of which relating to phosphate analogs are incorporated herein by reference. In some embodiments, an oligonucleotide provided herein comprises a 4′-phosphate analog at a 5′-terminal nucleotide. In some embodiments, a phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the aminomethyl group is bound to the 4′-carbon of the sugar moiety or analog thereof. In certain embodiments, a 4′-phosphate analog is an oxymethylphosphonate. In some embodiments, an oxymethylphosphonate is represented by the formula —O—CH₂—PO(OH)₂ or —O—CH₂—PO(OR)₂, in which R is independently selected from H, CH₃, an alkyl group, CH₂CH₂CN, CH₂OCOC(CH₃)₃, CH₂OCH₂CH₂Si (CH₃)₃, or a protecting group. In certain embodiments, the alkyl group is CH₂CH₃. More typically, R is independently selected from H, CH₃, or CH₂CH₃. In some embodiments, the 5′-terminal modification is to the sense strand. In some embodiments, the 5′-terminal modification is to the antisense strand.

In some embodiments, a phosphate analog attached to the oligonucleotide is a methoxy phosphonate (MOP). In some embodiments, a phosphate analog attached to the oligonucleotide is a 5′ mono-methyl protected MOP. In some embodiments, the following uridine nucleotide comprising a phosphate analog may be used, e.g., at the first position of an antisense (guide) strand:

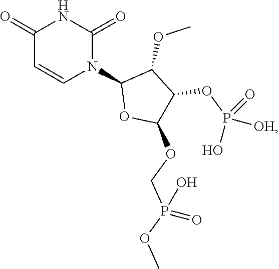 [SMILES: CO[C@@H]1[C@H](OP(=O)(O)O)[C@@H](OCP(=O)(O)OC)O[C@H]1n1ccc(=O)[nH]c1=O]

which modified nucleotide is referred to as [MePhosphonate-4O-mU] or 5′-Methoxy, Phosphonate-4′oxy-2′-O-methyluridine.

### c. Modified Internucleoside Linkages

In some embodiments, an oligonucleotide may comprise a modified internucleotide linkage. In some embodiments, phosphate modifications or substitutions may result in an oligonucleotide that comprises at least one (e.g., at least 1, at least 2, at least 3 or at least 5) modified internucleotide linkage. In some embodiments, any one of the oligonucleotides disclosed herein comprises 1 to 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9, or 10 modified internucleotide linkages.

A modified internucleotide linkage may be a phosphorodithioate linkage, a phosphorothioate linkage, a phosphotriester linkage, a thionoalkylphosphonate linkage, a thionalkylphosphotriester linkage, a phosphoramidite linkage, a phosphonate linkage or a boranophosphate linkage. In some embodiments, at least one modified internucleotide linkage of the oligonucleotides is a phosphorothioate linkage. In some embodiments, the internucleotide linkage is a 4-O-methylene phosphonate linkage of the following structure:

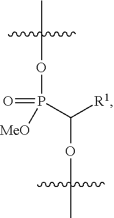

wherein R′ is H or a C₁-C₄ alkyl group. In some embodiments, R′ is methyl.

In some embodiments, the oligonucleotide has a phosphorothioate linkage between nucleotides at one or more of positions 1 and 2 of the sense strand (i.e., at the 5′-terminal region), positions 1 and 2 of the antisense strand, positions 2 and 3 of the antisense strand, positions 3 and 4 of the antisense strand (i.e., at the 5′-terminal region), positions 20 and 21 of the antisense strand, and positions 21 and 22 of the antisense strand (i.e., 3′-terminal region). In some embodiments, the oligonucleotide has a phosphorothioate linkage between nucleotides at each of positions 1 and 2 of the sense strand, positions 1 and 2 of the antisense strand, positions 2 and 3 of the antisense strand, positions 20 and 21 of the antisense strand, and positions 21 and 22 of the antisense strand.

### d. Base Modifications

In some embodiments, oligonucleotides provided herein have one or more modified nucleobases. In some embodiments, modified nucleobases (also referred to herein as base analogs) are linked at the 1′ position of a nucleotide sugar moiety. In certain embodiments, a modified nucleobase is a nitrogenous base. In certain embodiments, a modified nucleobase does not contain nitrogen atom (see, e.g., U.S. patent publication no. 20080274462). In some embodiments, a modified nucleotide comprises a universal base. However, in certain embodiments, a modified nucleotide does not contain a nucleobase (abasic).

In some embodiments, a universal base is a heterocyclic moiety located at the 1′ position of a nucleotide sugar moiety in a modified nucleotide, or the equivalent position in a nucleotide sugar moiety substitution, that, when present in a duplex, can be positioned opposite more than one type of base without substantially altering structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. However, in some embodiments, compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include inosine, 1-β-D-ribofuranosyl-5-nitroindole, and/or 1-β-D-ribofuranosyl-3-nitropyrrole (U.S. patent publication no. 20070254362; Van Aerschot et al., NUCLEIC ACIDS RES., 1995, 23 (21):4363-70; Loakes et al., NUCLEIC ACIDS RES. 1995, 23 (13):2361-6; Loakes and Brown, NUCLEIC ACIDS RES., 1994, 22 (20):4039-43. Each of the foregoing is incorporated by reference herein for their disclosures relating to base modifications).

### e. Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

In some embodiments, a reversibly modified nucleotide comprises a glutathione-sensitive moiety. Typically, nucleic acid molecules have been chemically modified with cyclic disulfide moieties to mask the negative charge created by the internucleotide diphosphate linkages and improve cellular uptake and nuclease resistance (see U.S. patent publication 20110294869, International patent publication WO2015188197; Meade et al., NATURE BIOTECH., 2014,32:1256-1263; International patent publication WO2014088920; each of which are incorporated by reference for their disclosures of such modifications). This reversible modification of the internucleotide diphosphate linkages is designed to be cleaved intracellularly by the reducing environment of the cytosol (e.g., glutathione). Earlier examples include neutralizing phosphotriester modifications that were reported to be cleavable inside cells (Dellinger et al., J. AM. CHEM. SOC., 2003, 125:940-950).

In some embodiments, such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed, and the result is a cleaved oligonucleotide. Using reversible, glutathione sensitive moieties, it is possible to introduce sterically larger chemical groups into the oligonucleotide of interest as compared to the options available using irreversible chemical modifications. These larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity, and reduced immunogenicity. In some embodiments, the structure of the glutathione-sensitive moiety can be engineered to modify the kinetics of its release.

In some embodiments, a glutathione-sensitive moiety is attached to the sugar of the nucleotide. In some embodiments, a glutathione-sensitive moiety is attached to the 2′ carbon of the sugar of a modified nucleotide. In some embodiments, the glutathione-sensitive moiety is located at the 5-carbon of a sugar, particularly when the modified nucleotide is the 5′-terminal nucleotide of the oligonucleotide. In some embodiments, the glutathione-sensitive moiety is located at the 3-carbon of sugar, particularly when the modified nucleotide is the 3′-terminal nucleotide of the oligonucleotide. In some embodiments, the glutathione-sensitive moiety comprises a sulfonyl group (see, e.g., International patent publication WO2018039364, the contents of which are incorporated by reference herein for its relevant disclosures).

### f. Targeting Ligands

In some embodiments, it may be desirable to target the oligonucleotides of the disclosure to one or more cells or one or more organs. Such a strategy may help to avoid undesirable effects in other organs or may avoid undue loss of the oligonucleotide to cells, tissue or organs that would not benefit from the oligonucleotide. Accordingly, in some embodiments, oligonucleotides disclosed herein may be modified to facilitate targeting of a particular tissue, cell or organ, e.g., to facilitate delivery of the oligonucleotide to the liver. In certain embodiments, oligonucleotides disclosed herein may be modified to facilitate delivery of the oligonucleotide to the hepatocytes of the liver. In some embodiments, an oligonucleotide comprises a nucleotide that is conjugated to one or more targeting ligands.

A targeting ligand may comprise a carbohydrate, amino sugar, cholesterol, peptide, polypeptide, protein or part of a protein (e.g., an antibody or antibody fragment) or lipid. In some embodiments, a targeting ligand is an aptamer. For example, a targeting ligand may be an RGD peptide that is used to target vasculature or glioma cells, CREKA peptide to target tumor vasculature or stoma, transferring, lactoferrin, or an aptamer to target transferrin receptors expressed on CNS vasculature, or an anti-EGFR antibody to target EGFR on glioma cells. In some embodiments, the targeting ligand is one or more GalNAc moieties.

In some embodiments, 1 or more (e.g., 1, 2, 3, 4, 5 or 6) nucleotides of an oligonucleotide are each conjugated to a separate targeting ligand. In some embodiments, 2 to 4 nucleotides of an oligonucleotide are each conjugated to a separate targeting ligand. In some embodiments, targeting ligands are conjugated to 2 to 4 nucleotides at either ends of the sense or antisense strand (e.g., ligand are conjugated to a 2 to 4 nucleotide overhang or extension on the 5′ or 3′ end of the sense or antisense strand) such that the targeting ligands resemble bristles of a toothbrush and the oligonucleotide resembles a toothbrush. For example, an oligonucleotide may comprise a stem-loop sequence at either the 5′ or 3′ end of the sense strand and 1, 2, 3 or 4 nucleotides of the loop of the stem may be individually conjugated to a targeting ligand.

In some embodiments, the targeting ligand is a GalNAc moiety. GalNAc is a high affinity ligand for asialoglycoprotein receptor (ASGPR), which is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins). Conjugation (either indirect or direct) of GalNAc moieties to oligonucleotides can be used to target these oligonucleotides to the ASGPR expressed on cells.

In some embodiments, an oligonucleotide is conjugated directly or indirectly to a monovalent GalNAc. In some embodiments, the oligonucleotide is conjugated directly or indirectly to more than one monovalent GalNAc (i.e., is conjugated to 2, 3, or 4 monovalent GalNAc moieties, and is typically conjugated to 3 or 4 monovalent GalNAc moieties). In some embodiments, an oligonucleotide of the instant disclosure is conjugated to a one or more bivalent GalNAc, trivalent GalNAc, or tetravalent GalNAc moieties.

In some embodiments, 1 or more (e.g., 1, 2, 3, 4, 5 or 6) nucleotides of an oligonucleotide are each conjugated to a GalNAc moiety. In some embodiments, 2 to 4 nucleotides of tetraloop are each conjugated to a separate GalNAc. In some embodiments, 1 to 3 nucleotides of triloop are each conjugated to a separate GalNAc. In some embodiments, targeting ligands are conjugated to 2 to 4 nucleotides at either ends of the sense or antisense strand (e.g., ligands are conjugated to a 2 to 4 nucleotide overhang or extension on the 5′ or 3′ end of the sense or antisense strand) such that the GalNAc moieties resemble bristles of a toothbrush and the oligonucleotide resembles a toothbrush. In some embodiments, GalNAc moieties are conjugated to a nucleotide of the sense strand. For example, four GalNAc moieties can be conjugated to nucleotides in the tetraloop of the sense strand where each GalNAc moiety is conjugated to one nucleotide.

In some embodiments, an oligonucleotide herein comprises a monovalent GalNAc attached to a guanidine nucleotide, referred to as [ademG-GalNAc] or 2′-aminodiethoxymethanol-Guanidine-GalNAc, as depicted below:

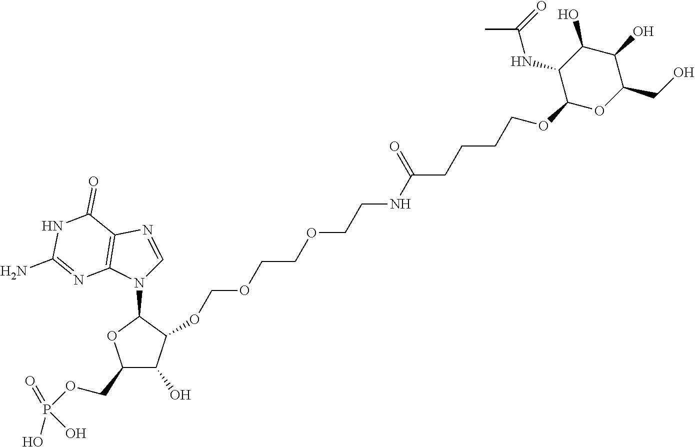 [SMILES: CC(=O)N[C@H]1[C@H](OCCCCC(=O)NCCOCCOCO[C@@H]2[C@H](O)[C@@H](COP(=O)(O)O)O[C@H]2n2cnc3c(=O)[nH]c(N)nc32)O[C@H](CO)[C@H](O)[C@@H]1O]

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below.

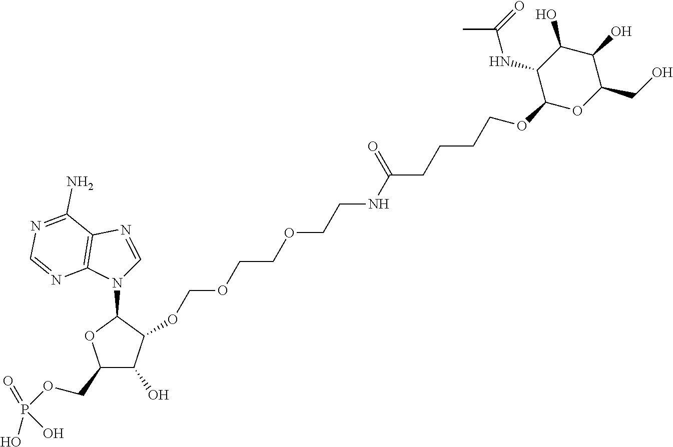

An example of such conjugation is shown below for a loop comprising from 5′ to 3′ the nucleotide sequence GAAA (Z=linker, X=heteroatom) stem attachment points are shown. In the chemical formula,

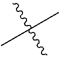

is used to describe an attachment point to the oligonucleotide strand.

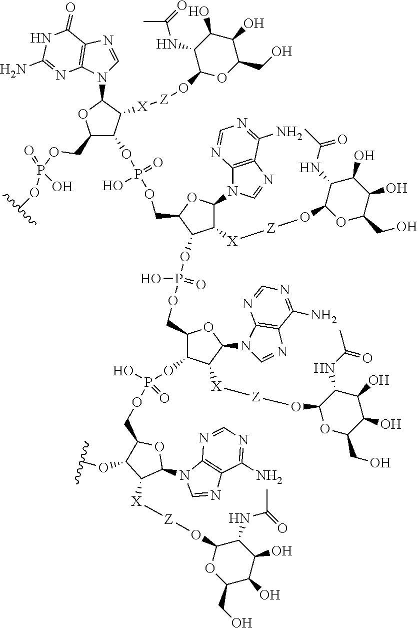

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in International patent publication WO2016100401, the contents of which relating to such linkers are incorporated herein by reference. In some embodiments, the linker is a labile linker. However, in other embodiments, the linker is stable. A “labile linker” refers to a linker that can be cleaved, e.g., by acidic pH. A “stable linker” refers to a linker that cannot be cleaved.

An example is shown below for a loop comprising from 5′ to 3′ the nucleotides GAAA, in which GalNAc moieties are attached to nucleotides of the loop using an acetal linker. In the chemical formula,

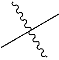

is an attachment point to the oligonucleotide strand.

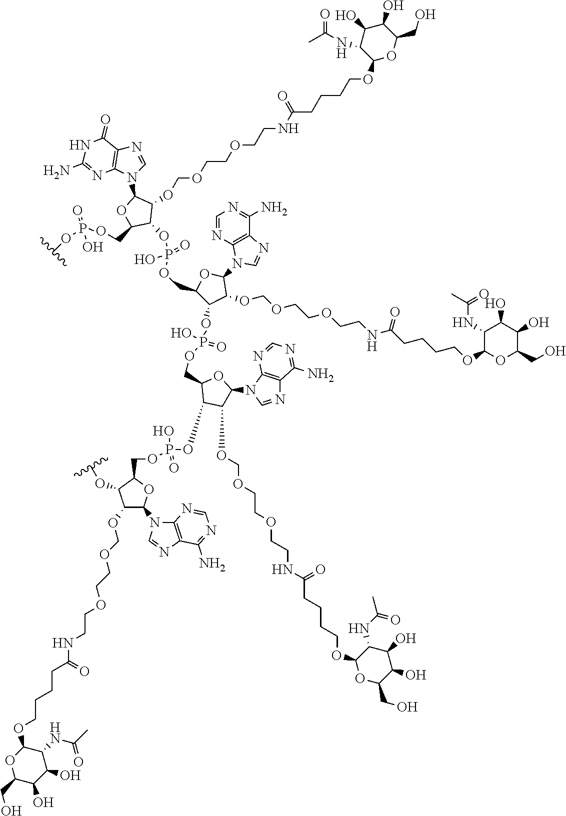 [SMILES: *O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(N)ncnc32)O[C@@H]1COP(=O)(O)O[C@H]1[C@@H](OCOCCOCCNC(=O)CCCCO[C@@H]2O[C@H](CO)[C@H](O)[C@H](O)[C@H]2NC(C)=O)[C@H](n2cnc3c(=O)[nH]c(N)nc32)O[C@@H]1COP(=O)(O)O*]

Any appropriate method or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in International Patent Application Publication Number WO2016100401, the contents of which relating to such linkers are incorporated herein by reference. In some embodiments, the linker is a labile linker. However, in other embodiments, the linker is stable. A “fairly stable linker” refers to a linker that cannot be cleaved.

### g. Modification Patterns

In some embodiments, the oligonucleotide is modified to increase resistance to phosphatases, nucleases, and other enzymes; enhance or maintain hybridization stability; provide targeting specificity; and where appropriate, enhance RNA silencing processing (e.g., via Dicer and Argonaut). In some embodiments, each of the modifications of the sugar moiety, 5′-terminal phosphate, internucleoside linkage, and base, and reversible modifications and modification via targeting ligands are incorporated into the oligonucleotide in defined embodiments.

In some embodiments, the 5′-terminal phosphate of the sense strand is modified with phosphate analogs, e.g., with phosphorothioate or 4′-phosphate analogs. In some embodiments, the internucleotide linkage of nucleotides at nucleotide positions 1 and 2 of the sense strand are modified, e.g., with a phosphorothioate or 4′-phosphate analogs. In some embodiments, the 5′-terminal phosphate of the sense strand is modified with a 4′-O-methyl phosphonate.

In some embodiments, the 5′-terminal phosphate or the internucleotide linkage of the 5′-terminal nucleotides of the antisense strand is modified, e.g., with a phosphorothioate. In some embodiments, the internucleotide linkages of the nucleotides at nucleotide positions 1 and 2, and 2 and 3, and optionally positions 3 and 4 of the antisense strand are modified, e.g., with a phosphorothioate.

In some embodiments, the sense strand has one or more, up to 4 nucleotides at nucleotide positions 7 to 10, preferably nucleotide positions 9, 10, 11, with the sugar moiety modified with a 2′-F. In some embodiments, the sense strand has the nucleotides at nucleotide positions 9, 10, 11 with the sugar moiety modified with a 2′-F. In the foregoing embodiments, the remaining nucleotides of the sense stand have the sugar moiety modified with a 2′-O-methyl.

In some embodiments, the antisense strand has one or more, up to 6, up to 5, up to 4 or up to 3 of the nucleotides at nucleotide positions 1, 2, 3, 5, 6, 7, 10, 14, and 16 with the sugar moiety modified with a 2′-F. In some embodiments, the antisense strand has at least the nucleotide at nucleotide positions 5 or 14, or both nucleotide positions 5 and 14, with the sugar moiety modified with a 2′-F. In some embodiments, the antisense strand has at least the nucleotide at nucleotide position 5, and optionally up to 5 nucleotides of the nucleotides at nucleotide positions 1, 2, 3, 6, 7, 10, 14, and 16, with the sugar moiety modified with a 2′-F. In some embodiments, the antisense strand has at least the nucleotide at nucleotide position 14, and optionally up to 5 of the nucleotides at nucleotide positions 1, 2, 3, 5, 6, 7, 10, 14, and 16, with the sugar moiety modified with a 2′-F. In some embodiments, the antisense strand has at least the nucleotide at nucleotide positions 5 and 14, and optionally up to 4 of the nucleotides at nucleotide positions 1, 2, 3, 6, 7, 10, and 16, with the sugar moiety modified with a 2′-F. In some embodiments, the antisense strand has nucleotides at nucleotide positions 2, 5 and 14 with the sugar moiety modified with a 2′-F. In the foregoing embodiments, the remaining nucleotides of the antisense strand have the sugar moiety modified with a with a 2′-O-methyl.

In some embodiments, one or more of the following positions are modified with a 2′-O-methyl: positions 1-7, or 12-36 of the sense strand and/or positions 1, 6, 8, 9, 11-13, or 15-22 of the antisense strand; and wherein one or more of the following positions are modified with a 2′-fluoro: positions 8-11 of the sense strand and/or positions 2, 3, 4, 5, 7, 10, or 14 of the antisense strand. In certain embodiments, positions 1-7, or 12-36 of the sense and positions 1, 6, 8, 9, 11-13, or 15-22 of the antisense strand are modified with a 2′-O-methyl; and positions 8-11 of the sense strand and positions 2, 3, 4, 5, 7, 10, or 14 of the antisense strand are modified with a 2′-fluoro.

In some embodiments, the oligonucleotide having a sense strand and an antisense strand, further contains a stem-loop sequence S1-L-S2, wherein the L is a tetraloop, pentaloop or triloop. In some embodiments, one or more, up to all of the nucleotides of S1 and S2 region have the sugar moiety modified with a 2′-O-methyl. In some embodiments, the 5′-terminal nucleotide of the L sequence has a modification of the sugar moiety with a 2′-O-methyl, and the remaining nucleotides of L have a targeting ligand. In some embodiments, all the nucleotides of the L region have a targeting ligand, e.g., GalNAc.

In some embodiments, the antisense strand has a 3′ overhang of 1 to 2 nucleotides, preferably a 2-nucleotide overhang, when the sense strand and the antisense strand form a duplex. In some embodiments, nucleotides of the 3′ overhang of the antisense strand have a modification of the internucleotide linkage, e.g., phosphorothioate. By way of example and not limitation, an antisense strand of 22 nucleotides in length have the internucleoside linkage between residues 20 and 21, and 21 and 22 modified with a phosphorothioate linkage.

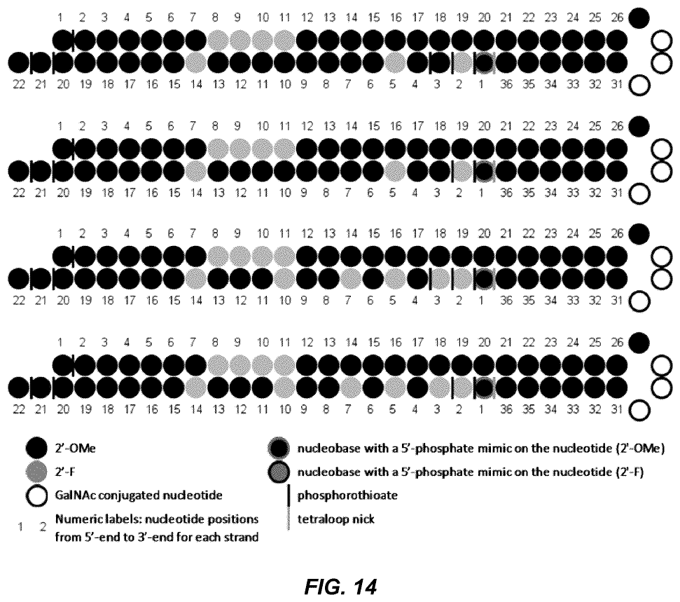

In some embodiments, by way of example and not limitation, for an exemplary oligonucleotide of the structure shown in FIG. 14 (e.g., sense strand of 36 nucleotides in length and an antisense of 22 nucleotides in length, where the sense strand contains a stem-loop sequence comprising regions S1-L-S2), the pattern of modifications of the nucleotides are described in FIG. 14.

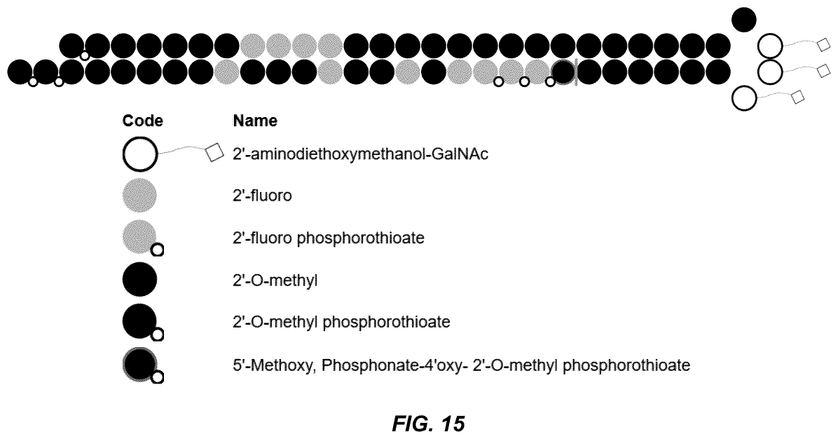

In some embodiments, by way of example and not limitation, for an exemplary oligonucleotide of the structure shown in FIG. 15 (e.g., sense strand of 36 nucleotides in length and an antisense of 22 nucleotides in length, where the sense strand contains a stem-loop sequence comprising regions S1-L-S2), the pattern of modifications of the nucleotides is described in FIG. 15.

In some embodiments, the oligonucleotides of the present disclosure include other variations of modifications patterns that incorporate modifications of the sugar moiety, modifications of the 5′-terminal phosphate, modifications of internucleoside linkages, modifications of the base, reversible modifications, and modifications via targeting ligands as described herein.

In some embodiments, an oligonucleotide of the present disclosure is a sense strand, an antisense strand, or a double stranded oligonucleotide selected from Table A.

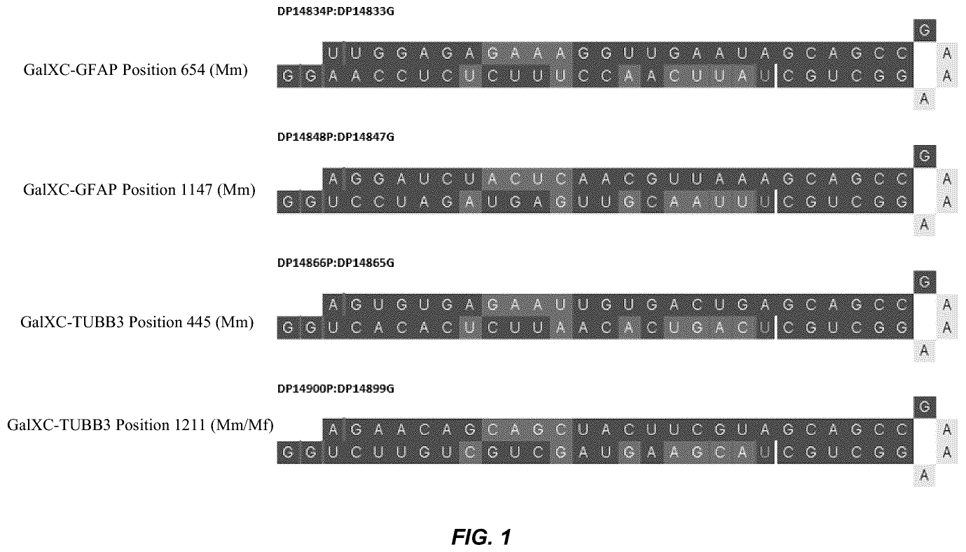

TABLE A
Sequence and modification information for the oligonucleotides depicted in FIG. 1.

| GalXC | | | |
|---|---|---|---|
| passenger: | Modification Pattern | | Corresponding unmodified |
| Guide | (5′ to 3′) | Modified Sequence (5′ to 3′) | sequence (5′ to 3′) |
| GalXC- | {MS}MMMMMMFF | [mUs][mU][mG][mG][mA][mG][mA][fG][fA][fA][fA][mG][mG] | UUGGAGAGAAAGGUUGAA |
| GFAP | FFMMMMMMMMM | [mU][mU][mG][mA][mA][mU][mA][mG][mC][mA][mG][mC][mC] | UAGCAGCCGAAAGGCUGC |
| Position | MMMMMMM[adem- | [mG][ademA-GalNAc][ademA-GalNAc][ademA-GalNAc][mG][mG] | (SEQ ID NO. 1) |
| 654 | GalNAc][adem- | [mC][mU][mG][mC] | |
| (Mm) | GalNAc][adem- | | |
| | GalNAc]MMMMMM | | |
| | {MePhosphonate-4O- | [MePhosphonate-4O- | UAUUCAACCUUUCUCUCCA |
| | MS}{FS}{FS}FFMF | MUs][fAs][fUs][fU][fC][mA][fA][mC][mC][fU][mU][mU][mC][fU] | AGG |
| | MMFMMMFMMMM | [mC][mU][mC][mC][mA][mAs][mGs][mG] | (SEQ ID NO. 2) |
| | M{MS}{MS}M | | |
| GalXC- | {MS}MMMMMMFFF | [mAs][mG][mG][mA][mU][mC][mU][fA][fC][fU][fC][mA][mA][mC] | AGGAUCUACUCAACGUUAA |
| GFAP | FMMMMMMMMMM | [mG][mU][mU][mA][mA][mA][mG][mC][mA][mG][mC][mC][mG] | AGCAGCCGAAAGGCUGC |
| Position | MMMMMM[adem- | [ademA-GalNAc][ademA-GalNAc][ademA-GalNAc][mG][mG][mC] | (SEQ ID NO. 3) |
| 1147 | GalNAc][adem- | [mU][mG][mC] | |
| (Mm) | GalNAc][adem- | | |
| | GalNAc]MMMMMM | | |
| | {MePhosphonate-4O- | [MePhosphonate-4O- | UUUAACGUUGAGUAGAUC |
| | MS}{FS}{FS}FFMFM | MUs][fUs][fUs][fA][fA][mC][fG][mU][mU][fG][mA][mG][mU][fA] | CUGG |
| | MFMMMFMMMMM | [mG][mA][mU][mC][mC][mUs][mGs][mG] | (SEQ ID NO. 4) |
| | {MS}{MS}M | | |
| GalXC- | {MS}MMMMMMFFF | [mAs][mG][mU][mG][mU][mG][mA][fG][fA][fA][fU][mU][mG][mU] | AGUGUGAGAAUUGUGACU |
| TUBB3 | FMMMMMMMMMM | [mG][mA][mC][mU][mG][mA][mG][mC][mA][mG][mC][mC][mG] | GAGCAGCCGAAAGGCUGC |
| Position | MMMMMM[adem- | [ademA-GalNAc][ademA-GalNAc][ademA-GalNAc][mG][mG][mC] | (SEQ ID NO. 5) |
| 445 | GalNAc][adem- | [mU][mG][mC] | |
| (Mm) | GalNAc][adem- | | |
| | GalNAc]MMMMMM | | |
| | {MePhosphonate-4O- | [MePhosphonate-4O- | UCAGUCACAAUUCUCACAC |
| | MS}{FS}{FS}FFMFM | MUs][fCs][fAs][fG][fU][mC][fA][mC][mA][fA][mU][mU][mC][fU] | UGG |
| | MFMMMFMMMMM | [mC][mA][mC][mA][mC][mUs][mGs][mG] | (SEQ ID NO. 6) |
| | {MS}{MS}M | | |
| GalXC- | {MS}MMMMMMFFFF | [mAs][mG][mA][mA][mC][mA][mG][fC][fA][fG][fC][mU][mA][mC] | AGAACAGCAGCUACUUCGU |
| TUBB3 | MMMMMMMMMMM | [mU][mU][mC][mG][mU][mA][mG][mC][mA][mG][mC][mC][mG] | AGCAGCCGAAAGGCUGC |
| Position | MMMMM[adem- | [ademA-GalNAc][ademA-GalNAc][ademA-GalNAc][mG][mG][mC] | (SEQ ID NO. 7) |
| 1211 | GalNAc][adem- | [mU][mG][mC] | |
| (Mm/Mf) | GalNAc][adem- | | |
| | GalNAc]MMMMMM | | |
| | {MePhosphonate-4O- | [MePhosphonate-4O- | UACGAAGUAGCUGCUGUUC |
| | MS}{FS}{FS}FFMFM | MUs][fAs][fCs][fG][fA][mA][fG][mU][mA][fG][mC][mU][mG][fC] | UGG |
| | MFMMMFMMMMM | [mU][mG][mU][mU][mC][mUs][mGs][mG] | (SEQ ID NO. 8) |
| | {MS}{MS}M | | |

In the modification patterns of Table A:
“M” refers to a 2′-OMe modified nucleotide;
“F” refers to a 2′-F modified nucleotide;
“S” refers to a nucleotide with a 3′-phosphorothioate linkage;
“{MS}” refers to a 2′-OMe modified nucleotide with a 3′-phosphorothioate linkage;
“{FS}” refers to a 2′-F modified nucleotide with a 3′-phosphorothioate linkage;
“[adem-GalNAc]” refers to a nucleotide having a 2′-GalNAc conjugate: 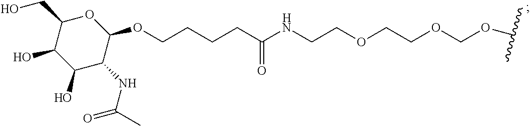 [SMILES: *OCOCCOCCNC(=O)CCCCO[C@@H]1O[C@H](CO)[C@H](O)[C@H](O)[C@H]1NC(C)=O]
“[MePhosphonate-4O-MS]” refers to a 5′-phosphonate-4′-Oxy-2′-OMe modified nucleotide with a 3′-phosphorothioate linkage: 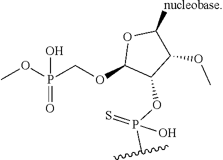
In the modified sequences of Table A:
“[mN]” refers to a 2′-OMe modified nucleotide;
“[fN]” refers to a 2′-F modified nucleotide;
“[mNs]” refers to a 2′-OMe modified nucleotide with a 3′-phosphorothioate linkage;
“[fNs]” refers to a 2′-F modified nucleotide with a 3′-phosphorothioate linkage;
“[ademA-GalNAc]” refers to an A nucleotide having a 2′-GalNAc conjugate: 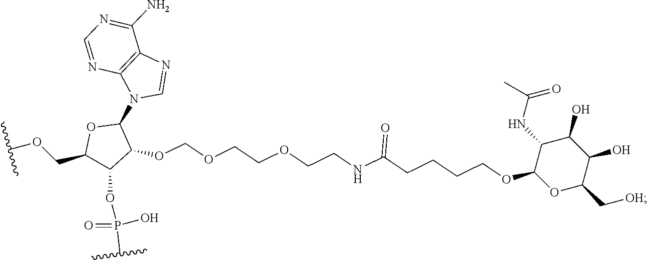 [SMILES: *OC[C@H]1O[C@@H](n2cnc3c(N)ncnc32)[C@H](OCOCCOCCNC(=O)CCCCO[C@@H]2O[C@H](CO)[C@H](O)[C@H](O)[C@H]2NC(C)=O)[C@@H]1OP(*)(=O)O]
“[MePhosphonate-4O-mUs]” refers to a 5′-phosphonate-4′-Oxy-2′-OMe uridine with a 3′-phosphorothioate linkage:

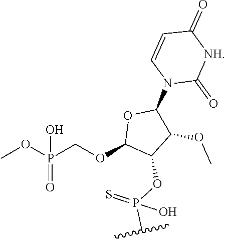 [SMILES: *P(O)(=S)O[C@@H]1[C@@H](OCP(=O)(O)OC)O[C@@H](n2ccc(=O)[nH]c2=O)[C@@H]1OC]

### III. Pharmaceutical Compositions

In some embodiments, an oligonucleotide is prepared as a pharmaceutical composition to facilitate use of the oligonucleotide. For example, oligonucleotides can be delivered to a subject or a cellular environment using a pharmaceutical composition or formulation that minimizes degradation, facilitates delivery and/or uptake, or provides another beneficial property to the oligonucleotides in the formulation. Such compositions can be suitably formulated such that when administered to a subject, either into the immediate environment of a target cell or to localized regions or organs, or when administered systemically, a sufficient portion of the oligonucleotides enter the cell to reduce levels of target RNA and/or a protein encoded by the target RNA. Any of a variety of suitable pharmaceutical compositions comprising an oligonucleotide can be used to deliver oligonucleotides for the reduction of RNA and/or protein expression in glial cells. In some embodiments, a pharmaceutical composition of an oligonucleotide comprises buffer solutions (e.g., phosphate buffered saline), liposomes, micellar structures, and capsids.

Formulations of oligonucleotides with cationic lipids can be used to facilitate transfection of the oligonucleotides into cells. For example, cationic lipids, such as lipofectin, cationic glycerol derivatives, and polycationic molecules (e.g., polylysine) can be used. Suitable lipids include Oligofectamine, Lipofectamine (Life Technologies), NC388 (Ribozyme Pharmaceuticals, Inc., Boulder, Colo.), or FuGene 6 (Roche) all of which can be used according to the manufacturer's instructions.

Accordingly, in some embodiments, a formulation comprises a lipid nanoparticle. In some embodiments, an excipient comprises a liposome, a lipid, a lipid complex, a microsphere, a microparticle, a nanosphere, or a nanoparticle, or may be otherwise formulated for administration to the cells, tissues, organs, or body of a subject in need thereof (see, e.g., REMINGTON: THE SCIENCE AND PRACTICE OF PHARMACY, 22nd Ed., Pharmaceutical Press, 2013).

In some embodiments, a pharmaceutical composition comprises an oligonucleotide of the disclosure, and a suitable excipient. In some embodiments, an excipient confers to a composition improved stability, improved absorption, improved solubility and/or therapeutic enhancement of the active ingredient. In some embodiments, an excipient is a buffering agent (e.g., sodium citrate, sodium phosphate, a tris base, or sodium hydroxide) or a vehicle (e.g., a buffered solution, petrolatum, dimethyl sulfoxide, or mineral oil). In some embodiments, an oligonucleotide is lyophilized for extending its shelf-life and then made into a solution before use (e.g., administration to a subject). Accordingly, in some embodiments, a pharmaceutical composition comprises an oligonucleotide described herein and an excipient which is a lyoprotectant (e.g., mannitol, lactose, polyethylene glycol, or polyvinylpyrrolidone), or a collapse temperature modifier (e.g., dextran, ficoll, or gelatin).

In some embodiments, a pharmaceutical composition is formulated to be compatible with its intended route of administration. Examples of routes of administration include parenteral, e.g., intravenous, intradermal, subcutaneous, oral (e.g., inhalation), transdermal, transmucosal, and rectal administration. In some embodiments, the pharmaceutical composition is formulated to be compatible with intrathecal, intraventrical, interstitial, intravenous, intranasal, or sublingual administration.

In some embodiments, the pharmaceutical composition is suitable for injectable use, such as formulated as sterile aqueous solutions (where water soluble) or dispersions, and sterile powders for the extemporaneous preparation of sterile injectable solutions or dispersion. In some embodiments for administration by injection, e.g., for intravenous, intrathecal, intraventrical, or interstitial, suitable carriers or excipients include, by way of example and not limitation, physiological saline, bacteriostatic water, Cremophor EL™ (BASF, Parsippany, N.J., USA) or phosphate buffered saline (PBS). The carrier can be a solvent or dispersion medium containing, for example, water, ethanol, polyol (for example, glycerol, propylene glycol, and liquid polyetheylene glycol, and the like), and suitable mixtures thereof. In some embodiments, it may be preferable to include isotonic agents, for example, sugars, polyalcohols such as mannitol, sorbitol, sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a pharmaceutical composition contains at least about 0.1% of the therapeutic agent (e.g., oligonucleotide targeting an RNA expressed in glial cells). In some embodiments, the percentage of the active ingredient(s) may be from about 1% to about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, sterile injectable solutions can be prepared by incorporating the active compound in the required amount in an appropriate solvent with one or a combination of excipients or carriers enumerated above, as suitable for injection, followed by filtered sterilization. Generally, dispersions are prepared by incorporating the active compound into a sterile vehicle, which contains a basic dispersion medium and other suitable ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

### IV. Uses of Oligonucleotides

In another aspect, the present disclosure provides a method for selective delivery of oligonucleotides of the disclosure to glial cells. In some embodiments, the selectivity for glial cell is in comparison to a neuronal cell. In some embodiments, a method of selective delivery of an oligonucleotide of the disclosure comprises contacting a glial cell with the oligonucleotide. In some embodiments, the glial cell is present in the nervous system of a subject. In some embodiments, a method for selective delivery of an oligonucleotide of the disclosure to a glial cell in a nervous system comprises administering the oligonucleotide to the nervous system of a subject. In some embodiments, the method for selective delivery of an oligonucleotide of the disclosure to a glial cell comprises administering the oligonucleotide to the nerve central nervous system or peripheral nervous system of a subject, as further discussed herein.

In some embodiments, the present disclosure provides a method for selective reduction in levels of a target RNA and/or protein encoded by the RNA expressed in a glial cell. In some embodiments, a method for selective reduction in levels of a target RNA and/or a protein encoded by the RNA expressed in a glial cell comprises contacting the glial cell with an effective amount an oligonucleotide disclosed herein, wherein the oligonucleotide comprises a sense strand and an antisense strand, and wherein the antisense strand comprises a region of complementarity to the target RNA. In some embodiments, a method for selective reduction in levels of a target RNA and/or a protein encoded by the RNA expressed in a glial cell comprises administering to the central nervous system or the peripheral nervous system of a subject an effective amount of an oligonucleotide of the disclosure, wherein the oligonucleotide comprises a sense strand and an antisense strand, and the antisense strand comprises a region of complementarity to the target RNA, wherein the oligonucleotide is effective in reducing the expression of the target RNA.

In some embodiments, the methods provided herein are applicable to any glial cell type. In some embodiments, the glial cell is an astrocyte, oligodendrocyte, ependymal cell, microglial cell, Schwann cell, satellite cell, or enteric glial cell. In some embodiments, the glial cell is an astrocyte, oligodendrocyte, ependymal cell, microglial cell, Schwann cell, satellite cell, or enteric glial cell present in the nervous system. In some embodiments, the astrocyte, oligodendrocyte, ependymal cell, microglial cell, or satellite cell is present in the central nervous system. In some embodiments, the astrocyte, oligodendrocyte, ependymal cell, microglial cell, Schwann cell, satellite cell, or enteric glial cell is present in the peripheral nervous system.

In some embodiments, the methods provide for selective delivery of an oligonucleotide of the disclosure to an astrocyte, oligodendrocyte, ependymal cell, microglial cell, Schwann cell, satellite cell, or enteric glial cell. In some embodiments, the methods provide for selective delivery of an oligonucleotide of the disclosure to an astrocyte. In some embodiments, the methods provide for selective delivery of an oligonucleotide of the disclosure to an oligodendrocyte. In some embodiments, the methods provide for selective delivery of an oligonucleotide of the disclosure to an ependymal cell. In some embodiments, the methods provide for selective delivery of an oligonucleotide of the disclosure to a microglial cell. In some embodiments, the methods provide for selective delivery of an oligonucleotide of the disclosure to a Schwann cell. In some embodiments, the methods provide for selective delivery of an oligonucleotide of the disclosure to a satellite cell. In some embodiments, the methods provide for selective delivery of an oligonucleotide of the disclosure to an enteric glial cell.

In some embodiments, the methods provide for selective delivery of an oligonucleotide of the disclosure to an astrocyte, oligodendrocyte, ependymal cell, microglial cell, or satellite cell in the central nervous system of a subject. In some embodiments, the central nervous system includes the brain and spinal cord. In some embodiments, the methods provide for selective delivery of an oligonucleotide of the disclosure to a glial cell in the brain or brain stem, including glial cells in localized regions of the brain or brainstem. In some embodiments, the methods provide for selective delivery of an oligonucleotide of the disclosure to a glial cell in the frontal cortex, striatum, somatosensory cortex, hippocampus, hypothalamus, or cerebellum. In some embodiments, the methods provide for selective delivery of an oligonucleotide of the disclosure to a glial cell in the brainstem, such as the pons or medulla.

In some embodiments, the methods provide for selective delivery of an oligonucleotide of the disclosure to a glial cell in the spinal cord of a subject, including localized regions of the spinal cord. In some embodiments, the methods provide for selective delivery of an oligonucleotide of the disclosure to a glial cell in the cervical spinal cord, thoracic spinal cord, or lumbar spinal cord.

In some embodiments, the glial cell in the central nervous system is an astrocyte, oligodendrocyte, ependymal cell, microglial cell, or satellite cell. Accordingly, in some embodiments, the methods provide for selective delivery of an oligonucleotide of the disclosure to an astrocyte in the central nervous system of a subject. In some embodiments, the methods provide for selective delivery of an oligonucleotide of the disclosure in an oligodendrocyte in the central nervous system of a subject. In some embodiments, the methods provide for selective delivery of an oligonucleotide of the disclosure to an ependymal cell in the central nervous system of a subject. In some embodiments, the methods provide for selective delivery of an oligonucleotide of the disclosure to a microglial cell in the central nervous system of a subject. In some embodiments, the methods provide for selective delivery of an oligonucleotide of the disclosure to a satellite cell in the central nervous system of a subject.

In some embodiments, the methods provide for selective delivery of an oligonucleotide of the disclosure to an astrocyte, oligodendrocyte, ependymal cell, microglial cell, Schwann cell, satellite cell, or enteric glial cell in the peripheral nervous system of a subject. In some embodiments, the glial cell in the peripheral nervous system includes those localized in, among others, cranial nerves, spinal nerves, and peripheral nerves. In some embodiments, the glial cell in the peripheral nervous system includes, among others, Schwann cell, satellite cell, and enteric glial cell. In some embodiments, the methods provide for selective delivery of an oligonucleotide of the disclosure to a Schwann cell in the peripheral nervous system. In some embodiments, the methods provide for selective delivery of an oligonucleotide of the disclosure to a satellite cell in the peripheral nervous system. In some embodiments, the methods provide for selective delivery of oligonucleotide of the disclosure to an enteric glial cell in the peripheral nervous system.

In some embodiments, the methods provide for selective reduction in levels of a target RNA and/or protein encoded by the RNA expressed in an astrocyte, oligodendrocyte, ependymal cell, microglial cell, Schwann cell, satellite cell, or enteric glial cell. In some embodiments, the methods provide for selective reduction in levels of a target RNA and/or a protein encoded by the RNA expressed in an astrocyte. In some embodiments, the methods provide for selective reduction in levels of a target RNA and/or a protein encoded by the RNA expressed in an oligodendrocyte. In some embodiments, the methods provide for selective reduction in levels of a target RNA and/or a protein encoded by the RNA expressed in an ependymal cell. In some embodiments, the methods provide for selective reduction in levels of a target RNA and/or a protein encoded by the RNA expressed in a microglial cell. In some embodiments, the methods provide for selective reduction in levels of a target RNA and/or a protein encoded by the RNA expressed in a Schwann cell. In some embodiments, the methods provide for selective reduction in levels of a target RNA and/or a protein encoded by the RNA expressed in a satellite cell. In some embodiments, the methods provide for selective reduction in levels of a target RNA and/or a protein encoded by the RNA expressed in an enteric glial cell. In some embodiments, the methods provide for selective reduction in levels of a target RNA and/or a protein encoded by the RNA expressed in a mixture of glial cells.

In some embodiments, methods are provided for selective reduction in levels of a target RNA and/or protein encoded by the RNA expressed in a glial cell in the nervous system of a subject. In some embodiments, the methods provide for selective reduction in in levels of a target RNA and/or protein encoded by the RNA expressed in a glial cell in the central nervous system of a subject. In some embodiments, the central nervous system includes the brain and spinal cord. In some embodiments, the methods provide for selective reduction in levels of a target RNA and/or protein encoded by the RNA expressed in a glial cell in the brain or brain stem, including glial cells in localized regions of the brain or brainstem. In some embodiments, the methods provide for selective reduction in levels of a target RNA and/or protein encoded by the RNA expressed in a glial cell in the frontal cortex, striatum, somatosensory cortex, hippocampus, hypothalamus, or cerebellum. In some embodiments, the methods provide for selective reduction in levels of a target RNA and/or protein encoded by the RNA expressed in a glial cell in the brainstem, such as the pons or medulla.

In some embodiments, the methods provide for selective reduction in levels of a target RNA and/or protein encoded by the RNA expressed in a glial cell in the spinal cord of a subject, including a localized region of the spinal cord. In some embodiments, the methods provide for selective reduction in levels of a target RNA and/or protein encoded by the RNA expressed in a glial cell in the cervical spinal cord, thoracic spinal cord, or lumbar spinal cord.

In some embodiments, the glial cell in the central nervous system is an astrocyte, oligodendrocyte, ependymal cell, microglial cell, or satellite cell. In certain embodiments, the glial cell in the central nervous system is an astrocyte, ependymal cell, microglial cell, or satellite cell. In certain embodiments, the glial cells are astrocytes. In some embodiments, the methods provide for selective reduction in levels of a target RNA and/or protein encoded by the RNA expressed in an astrocyte in the central nervous system of a subject. In some embodiments, the methods provide for selective reduction in levels of a target RNA and/or protein encoded by the RNA expressed in an oligodendrocyte in the central nervous system of a subject. In some embodiments, the methods provide for selective reduction in levels of a target RNA and/or protein encoded by the RNA expressed in an ependymal cell in the central nervous system of a subject. In some embodiments, the methods provide for selective reduction in levels of a target RNA and/or protein encoded by the RNA expressed in a microglial cell in the central nervous system of a subject. In some embodiments, the methods provide for selective reduction in levels of a target RNA and/or protein encoded by the RNA expressed in a satellite cell in the central nervous system of a subject.

In some embodiments, the methods provide for selective reduction in levels of a target RNA and/or protein encoded by the RNA expressed in a glial cell in the peripheral nervous system of a subject. In some embodiments, the glial cell in the peripheral nervous system includes those localized in, among others, cranial nerves, spinal nerves, and peripheral nerves. In some embodiments, the glial cell in the peripheral nervous system includes, among others, Schwann cell, satellite cell, and enteric glial cell. In some embodiments, the methods provide for selective reduction in levels of a target RNA and/or protein encoded by the RNA expressed in a Schwann cell in the peripheral nervous system. In some embodiments, the methods provide for selective reduction in levels of a target RNA and/or protein encoded by the RNA expressed in a satellite cell in the peripheral nervous system. In some embodiments, the methods provide for selective reduction in levels of a target RNA and/or protein encoded by the RNA expressed in an enteric glial cell in the peripheral nervous system.

In some embodiments, a method for selective delivery to or selective reduction in levels of a target RNA and/or protein encoded by the RNA expressed in a glial cell in the nervous system of a subject comprises administering an effective amount of an oligonucleotide of the disclosure to a subject, wherein the administration is by means for delivering to the nervous system of the subject the effective amount of the oligonucleotide.

In some embodiments, a method for selective delivery to or selective reduction in levels of a target RNA and/or protein encoded by the RNA expressed in a glial cell in the nervous system of a subject comprises administering an effective amount of an oligonucleotide disclosed herein intrathecally, intraventrically, intravenously, interstitially, sublingually, or intranasally to a subject.

In some embodiments, a method of selective delivery to or selective reduction in levels of a target RNA and/or protein encoded by the RNA expressed in a glial cell in the central nervous system of a subject comprises intrathecally administering an effective amount of an oligonucleotide of the disclosure to the subject. In some embodiments, the glial cell is in the brain or spinal cord of the subject.

In some embodiments, a method of selective delivery to or selective reduction in levels of a target RNA and/or protein in a glial cell in the central nervous system of a subject comprises intraventrically administering an effective amount of an oligonucleotide of the disclosure to the subject.

In some embodiments, a method of selective delivery to or selective reduction in levels of a target RNA and/or protein encoded by the RNA in a glial cell in the central nervous system of a subject comprises interstitially administering an effective amount of an oligonucleotide of the disclosure to the subject. In some embodiments, the oligonucleotide is administered interstitially to localized regions in the central nervous system. In some embodiments, the oligonucleotide is administered interstitially to the frontal cortex, striatum, somatosensory cortex, hippocampus, hypothalamus, or cerebellum. In some embodiments, the oligonucleotide is administered interstitially to the spinal cord, including localized regions of the spinal cord, such as the cervical spinal cord, thoracic spinal cord, or lumbar spinal cord.

In some embodiments, a method of selective delivery to or selective reduction in levels of a target RNA and/or protein encoded by the RNA expressed in a glial cell in the central nervous system of a subject comprises intranasally administering an effective amount of an oligonucleotide of the disclosure to the subject. Intranasal administration exploits transport through the olfactory and/or trigeminal neural pathway to areas of the central nervous system, including the brain stem, cerebellum, spinal cord, olfactory bulb, and cortical and subcortical structures.

In addition to the above routes of administration, the oligonucleotides of the present disclosure can be administered sublingually or transdermally for delivery to the central nervous system through the trigeminal neural pathway. In some embodiments, the oligonucleotides can be administered intravenously where appropriate.

In some embodiments, oligonucleotides disclosed herein can be introduced using appropriate nucleic acid delivery methods including injection of a solution containing the oligonucleotides, bombardment by particles covered by the oligonucleotides, exposing the cell or organism to a composition containing the oligonucleotides, or electroporation of cell membranes in the presence of the oligonucleotides. Other appropriate methods for delivering oligonucleotides to cells can be used, such as lipid-mediated carrier transport, chemical-mediated transport, and cationic liposome transfection such as calcium phosphate, and others.

The effects of inhibition can be confirmed by an appropriate assay to evaluate one or more properties of a cell or subject, or by biochemical techniques that evaluate molecules indicative of RNA expression (e.g., RNA, protein). In some embodiments, the extent to which an oligonucleotide provided herein reduces levels of a target RNA expressed in a cell, tissue, or organ is evaluated by comparing expression levels (e.g., mRNA or protein levels) to an appropriate control (e.g., a level of RNA expression in a cell or population of cells to which an oligonucleotide has not been delivered or to which a negative control has been delivered). In some embodiments, an appropriate control level of RNAi expression may be a predetermined level or value, such that a control level need not be measured every time. The predetermined level or value can take a variety of forms. In some embodiments, a predetermined level or value can be single cut-off value, such as a median or mean.

In some embodiments, administration of an oligonucleotide as described herein results in a reduction in the level of RNA expression in a glial cell. In some embodiments, the reduction in levels of RNA expression may be a reduction to 1% or lower, 5% or lower, 10% or lower, 15% or lower, 20% or lower, 25% or lower, 30% or lower, 35% or lower, 40% or lower, 45% or lower, 50% or lower, 55% or lower, 60% or lower, 70% or lower, 80% or lower, or 90% or lower compared with an appropriate control level of RNA. In some embodiments, the reduction in levels of RNA expression is at least 50% or lower. In some embodiments, the reduction in levels of RNA expression is at least 70% or lower. In some embodiments, the reduction in levels of RNA expression is at least 80% or lower. In some embodiments, the appropriate control level may be a level of RNA expression in a glial cell or population of glial cells that have not been contacted with an oligonucleotide as described herein or treated with a negative control oligonucleotide (e.g., random nucleotide sequence). In some embodiments, the effect of delivery of an oligonucleotide to a glial cell according to a method disclosed herein is assessed after a finite period. For example, levels of RNA may be analyzed in a cell at least 8 hours, 12 hours, 18 hours, 24 hours; or at least one, two, three, four, five, six, seven, or fourteen days after introduction of the oligonucleotide into the cell.

In some embodiments, selective delivery to or selective reduction in levels of an RNA and/or protein encoded by the RNA expressed in a glial cell is in comparison to selective delivery or selective reduction of an another, perhaps closely related or alternate form of an RNA and/or protein expressed in a neuronal cell. In some embodiments, the selectivity or differential silencing is based on comparison of % reduction in expression of a target RNA expressed in both glial cell and neuronal cell. In some embodiments, the selectivity is based on comparison of % reduction in expression of glial cell specific RNA compared to the % reduction in expression of a neuronal cell specific RNA. In some embodiments, the selectivity for glial cell over neuronal cell is at least 1.2; 1.3; 1.4; 1.5; 1.6. 1.7, 1.8; 1.9 or 2 or higher. In some embodiments, the selectivity for glial cell over neuronal cell is 2 or greater; 2.5 or greater; 3 or greater; 3.5 or greater; 4 or greater; 4.5 or greater; or 5 or greater. In some embodiments, the selectivity is for delivery of an oligonucleotide of the disclosure to a glial cell as compared to a neuronal cell. In some embodiments, the selectivity is for reduction of an RNA that is specifically expressed in a glial cell compared to a RNA specifically expressed in a neuronal cell. In various embodiments, the RNA and/or protein expressed in the neuronal cell for use as a comparator can be any neuronal specific marker protein, such as described herein for neuronal cell markers. These neuronal specific markers include, among others, neuron specific enolase (NSE or gamma-enolase); neuronal nuclei (NeuN or Fox3); microtubule-associated protein 2 (MAP-2); Tubulin beta III (TUBB3); Doublecortin (DCX); c-fos; choline acetyltransferase (ChAT); and tyrosine hydroxylase. In some embodiments, assessing the selectivity between glial cell and neuronal uses siRNAs that produce similar % of reduction of the glial cell specific RNA expression and neuronal cell specific RNA expression in a non-neuronal cell, for example, a hepatocyte.

In some embodiments, an oligonucleotide is delivered in the form of a transgene that is engineered to express in a cell the oligonucleotides (e.g., its sense and antisense strands). In some embodiments, an oligonucleotide is delivered using a transgene that is engineered to express any oligonucleotide disclosed herein. Transgenes may be delivered using viral vectors (e.g., adenovirus, retrovirus, vaccinia virus, poxvirus, adeno-associated virus or herpes simplex virus) or non-viral vectors (e.g., plasmids or synthetic mRNAs). In some embodiments, transgenes can be injected directly to a subject.

In some embodiments herein, the oligonucleotide of the disclosure can selectively target any RNA expressed in a glial cell. In some embodiments, the antisense strand of the oligonucleotide of the disclosure comprises a region of complementarity to a target RNA expressed in a glial cell, wherein the oligonucleotide is effective in reducing expression of the target RNA of interest. As discussed above, in some embodiments, the target RNA of interest includes those whose expression in a glial cell is associated with a disease or disorder with glial cell dysfunction. In some embodiments, the antisense strand of the oligonucleotide comprises a region of complementarity to expressed RNA of: GFAP gene associated with Alexander Disease (AxD); PSAP gene associated with Metachromatic Leukodystrophy/Krabbe Disease; PMP22 gene associated with Charcot-Marie-Tooth disease; LMNB1 gene associated with Adult-Onset Leukodystrophy; APP gene and TAU (MAPT) gene associated with Alzheimer's Disease; SOD1 gene, C9orf72 gene, and HTT gene associated with Huntington's Disease; SNCA or ASYN gene and LRRK2 gene associated with Parkinson's Disease; ADK gene associated with Epilepsy; TNFα gene and ERK5/MAPK7 gene associated with Stroke; TNFα gene and GFAP gene associated with Traumatic Brain Injury and axonal injury; IL-1R2 gene associated with Autism; CD49d gene associated with Multiple Sclerosis; IGF-1 gene, EGF gene, TGF-β gene, and VEGF gene associated with Glioblastoma and glial-cell cancer; SOD1 gene, C9orf72 gene, and TDP-43 gene associated with amyotrophic lateral sclerosis (ALS); TNFα gene and CD38 gene associated with Neuroinflammation; ATXN2 gene, ATXN3 gene, and ATXN7 gene associated with Spinocerebellar Ataxias; TAU (MAPT) gene associated with Progressive Supranuclear Palsy; TAU (MAPT) gene associated with Primary age-related tauopathy (PART)/Neurofibrillary tangle-predominant senile dementia; TAU (MAPT) gene associated with Frontotemporal dementia and parkinsonism linked to chromosome 17 (FTDP-17); and EGR2 gene associated with Peripheral nerve demyelination.

In some embodiments, expression of genes containing mutated forms of the encoded proteins are targeted for reduction in expression. In some embodiments, genes whose overexpression is associated with the cause or manifestation of the disease or disorder is targeted for reduction in expression. In some embodiments, the gene(s) encoding mutated forms of a protein whose expression is associated with the disease or disorder are targeted for reduction in expression. In some embodiments, genes containing mutations or gene whose overexpression is associated with a disease or disorder include, by way of example and not limitation, the gene for GFAP (see, e.g., Rodriguez et al., AMERICAN JOURNAL OF HUMAN GENETICS, 2001, 69 (6):1413); PMP22 (see, e.g., Robaglia-Schlupp et al., BRAIN, 2002, 125 (10):2213-2221; Li et al., MOL NEUROBIOL., 2013, 47 (2):673-98); LMNB1 (see, e.g., Padiath, Q. S., Cell Dev. Biol., 2019, 7 (41):1-6); APP (see, e.g., Katsurabayashi et al., PHYSIOLOGICAL REPORTS, 2016, 4 (1):e12665); TAU/MAPT (see, e.g., Le Guennec et al., MOL PSYCHIATRY., 2017, 22 (8):1119-1125; SOD1 (see, e.g., Massenzio et al., BIOCHIMICA ET BIOPHYSICA Acta (BBA)—MOLECULAR BASIS OF DISEASE, 2018, 1864 (12):3771-3785), C9orf72 (see, e.g., Balendra et al., NAT REV NEUROL., 2018, 14 (9):544-558), ATXN2 (see, e.g., Ostrowski et al., GENES (Basel), 2017, 8 (6):157), ATXN3 (see, e.g., Neves-Carvalho et al., HUM MOL GENET, 2015, 24 (1):100-117), ATXN7 (see, e.g., Lan et al., MOL CELL BIOL., 2015, 35 (10):1777-1787); HTT (see, e.g., Shin et al., J CELL BIOL., 2005, 171 (6):1001-1012); SNCA/ASYN (see, e.g., Oliveira et al., CELL DEATH DIS., 2015, 6 (11):e1994); ADK (see, e.g., De Groot et al., EPILEPSIA, 2011, 53 (1):858-66), TNFα (see, e.g., Welser-Alves et al., NEUROCHEM INT., 2013, 63 (1):47-53); CD49d (see, e.g., Limmroth, V., NEUROLOGY, 2014, 83 (20):1780-1788); or TDP-43 (see, e.g., Lu et al., INT J BIOL SCI., 2016, 12 (9):1140-1149). The corresponding disease or disorder and the reference mRNA are described above.

In a further aspect, the present invention relates to a method for treating a subject having a disease or at risk of developing a disease caused by or associated with expression of a target gene in a glial cell. In some embodiments, disease or disorders that can be affected include, by way of example and not limitation, Alexander Disease (AxD) associated with expression of the GFAP gene; Metachromatic Leukodystrophy/Krabbe Disease associated with expression of the PSAP gene; Charcot-Marie-Tooth Disease associated with expression of PMP22 gene; Adult-Onset Leukodystrophy associated with expression of LMNB1 gene; Alzheimer's Disease associated with expression of the APP gene; Huntington's Disease associated with expression of SOD1 gene (e.g., reference mRNA sequence NM_000454.4), C9orf72 gene (e.g., reference mRNA sequence: NM_001256054.2; NM_018325.5; NM_145005.6), and HTT gene; Parkinson's Disease associated with expression of SNCA/ASYN gene and LRRK2 gene; epilepsy associated with expression of ADK gene; Stroke and its effects associated with expression of TNFα gene and ERK5/MAPK7 gene; traumatic brain injury and axonal injury and its effects associated with expression of TNFα gene and GFAP gene; autism associated with expression of IL-1R2 gene; multiple sclerosis associated with expression of CD49d gene; glioblastoma and glial-cell cancer associated with expression of IGF-1 gene, EGF gene, TGF-β gene, and VEGF gene; amyolateral sclerosis (ALS) associated with expression of SOD1 gene, C9orf72 gene, and TDP-43 gene; neuroinflammation associated with expression of TNFα gene and CD38 gene; spinocerebellar ataxias associated with expression of ATXN2 gene, ATXN3 gene, and ATXN7 gene; progressive supranuclear palsy associated with TAU (MAPT) gene; primary age-related tauopathy (PART)/neurofibrillary tangle-predominant senile dementia associated with expression of TAU (MAPT) gene; frontotemporal dementia and parkinsonism linked to chromosome 17 (FTDP-17) associated with expression of TAU (MAPT) gene; and peripheral nerve demyelination associated with expression of EGR2 gene.

In some embodiments, the methods described herein comprise administering to a subject an effective amount of an oligonucleotide, that is, an amount capable of producing a desirable therapeutic result. A therapeutically acceptable amount may be an amount that can treat a disease or disorder. The appropriate dosage for any one subject will depend on certain factors, including the subject's size, body surface area, age, the particular composition to be administered, the active ingredient(s) in the composition, time and route of administration, general health, and other drugs being administered concurrently.

As a non-limiting set of examples, the oligonucleotides of the instant disclosure would typically be administered quarterly (once every three months), bi-monthly (once every two months), monthly, or weekly. For example, the oligonucleotides may be administered every week or at intervals of two, or three weeks. The oligonucleotides may be administered daily.

In some embodiments, the subject to be treated is a human or non-human primate or other mammalian subject. Other exemplary subjects include domesticated mammals such as dogs and cats; livestock such as horses, cattle, pigs, sheep, goats, and chickens; and other mammals such as mice, rats, guinea pigs, and hamsters. In some embodiments, a human subject is referred to as a patient.

ii. Methods of Screening for Interfering Oligonucleotides for Selective Delivery and/or Selective Reduction of a Target RNA Expressed in Glial Cells.

In a further aspect, the present disclosure provides a method of screening for oligonucleotides that are selective for a glial cell or neuronal cell. In some embodiments, the screening is for identifying oligonucleotides that are selective for glial cells over neuronal cells. In some embodiments, the screening is for identifying oligonucleotides that are selective for neuronal cells over glial cells. In various embodiments, the oligonucleotide can reduce levels of a target RNA expressed in glial cells. In various embodiments, the oligonucleotide tested can reduce levels of a target RNA expressed in neuronal cells. In some embodiments, the oligonucleotide can reduce levels of an RNA that is expressed at higher levels in glial cells compared to neuronal cells. In some embodiments the oligonucleotide can reduce levels of an RNA expressed at higher levels in neuronal cells compared to glial cells.

In some embodiments, a method of screening for an oligonucleotide that selectively reduce levels of a target RNA expressed in glial cells or neuronal cells, comprises:
contacting a glial cell and a neuronal cell with a candidate oligonucleotide, wherein the candidate oligonucleotide comprises a region of complementarity to a target RNA expressed in both the glial cell and neuronal cell, wherein the oligonucleotide is capable of reducing expression of the target RNA; and
measuring the levels of target RNA in glial cells and neuronal cells to determine any differential effect on levels of target RNA expression.

In some embodiments, the method of screening for an oligonucleotide that selectively reduce levels of a target RNA expressed in glial cells or neuronal cells, comprises:
administering a candidate oligonucleotide to a nervous system of a subject, wherein the candidate oligonucleotide comprises a region of complementarity to a target RNA expressed in both glial cells and neuronal cells of the nervous system, wherein the oligonucleotide is capable of reducing expression of target RNA; and
measuring the levels of the target RNA in glial cells and neuronal cells to determine any differential effect on levels of target RNA expression. In some embodiments, the subject is a non-human mammal.

In some embodiments, an oligonucleotide displaying greater reduction in levels of the target RNA in glial cells over reduction in levels of the target RNA in neuronal cells is identified as being selective for glial cells. In some embodiments, the selectivity of the oligonucleotide for glial cells over neuronal cells is at least 1.5, 2, 2.5, 3, 3.5, 4, 4.5 or 5 or greater.

In some embodiments, an oligonucleotide displaying greater reduction in levels of the target RNA in neuronal cells over reduction in levels of the target RNA in glial cells is identified as being selective for neuronal cells. In some embodiments, the selectivity of the oligonucleotide for neuronal cells over glial cells is at least 1.5, 2, 2.5, 3, 3.5, 4, 4.5 or 5 or greater.

In some embodiments, the candidate oligonucleotide comprises a single stranded RNA or DNA, such as described herein.

In some embodiments, the oligonucleotide comprises a double stranded nucleic acid (dsNA), wherein the dsNA comprises a ribonucleotide. In some embodiments, the candidate oligonucleotide comprises a sense strand and an antisense strand, wherein the sense strand and the antisense strand forms a duplex and wherein the antisense strand has the region of complementarity to the target RNA and the oligonucleotide is capable of reducing levels of the target RNA.

In some embodiments, the candidate oligonucleotide is modified with one or more targeting ligands. In some embodiments, the targeting ligand comprises one or more of a carbohydrate, amino sugar, cholesterol, lipid, peptide, or mixtures thereof. In some embodiments, the targeting ligand comprises one or more GalNAc moieties, as described in detail herein.

In some embodiments, wherein the candidate oligonucleotide is administered to the nervous system of a subject, the measuring for determining the levels of target RNA in glial cells and neuronal cells is assessed by measuring the levels of the target RNA in glial cells and neuronal cells distributed throughout the nervous system or in localized regions of the nervous system. In some embodiments, the glial cell examined is an astrocyte, oligodendrocyte, ependymal cell, microglial cell, Schwann cell, satellite cell, enteric glial cell, or combinations thereof.

In some embodiments, a method of screening for interfering oligonucleotides that selectively reduce levels of a target RNA expressed in glial cells, or alternatively in neuronal cells, comprises:
contacting a glial cell with first candidate oligonucleotide, wherein the first candidate oligonucleotide comprises a region of complementarity to a first target RNA expressed in glial cells, wherein the first oligonucleotide is capable of reducing levels of first target RNA expression;
contacting a neuronal cell with a second candidate oligonucleotide, wherein the second candidate oligonucleotide comprises a region of complementarity to a second target RNA expressed in neuronal cells, wherein the second oligonucleotide is capable of reducing levels of second target RNA expression; and
measuring levels of first target RNA in glial cells and levels of second target RNA in neuronal cells and determining any differential reduction in first target RNA and second target RNA.

In some embodiments, a method of screening for oligonucleotides that selectively reduces levels of a target RNA expressed in glial cells, or alternatively neuronal cells, comprises:
administering a first candidate oligonucleotide to a nervous system of a first subject, wherein the first candidate oligonucleotide comprises a region of complementarity to a first target RNA expressed in glial cells, wherein the first oligonucleotide is capable of reducing expression of the first target RNA;
administering a second candidate oligonucleotide to a nervous system of a second subject, wherein the second candidate oligonucleotide comprises a region of complementarity to a second target RNA expressed in neuronal cells, wherein the first oligonucleotide is capable of reducing expression of the first target RNA; and
measuring levels of first target RNA in glial cells and second target RNA in neuronal cells in the nervous system of the first subject and second subject, wherein the first subject and the second subject are the same species.

In some embodiments, the first candidate oligonucleotide and the second candidate oligonucleotide differ only in regards to the region of complementary to the respective target RNAs. In some embodiments, the target RNA expressed in glial cells is specifically expressed in glial cells. In some embodiments, the target RNA expressed in neuronal cells is specifically expressed in neuronal cells. As used herein, “specifically expressed” refers to an RNA that is expressed in greater levels in one cell compared to another cell. In some embodiments, the target RNA is specifically expressed in one cell over the expression in another cell by 1.5, 2, 3, 4, 5, 6, 8, 10-fold or greater.

If a candidate oligonucleotide displays greater selectivity for one cell type over the other cell type, a greater reduction in levels of the target RNA (e.g., as % reduction) should be observed in the one cell over the reduction in levels of the target RNA in the other cell.

In some embodiments, an oligonucleotide displaying greater reduction in levels of the target RNA in glial cells over reduction in levels of the target RNA in neuronal cells is identified as being selective for glial cells. In some embodiments, the selectivity of the oligonucleotide for glial cells over neuronal cells is at least 1.5, 2, 2.5, 3, 3.5, 4, 4.5 or 5 or greater.

In some embodiments, an oligonucleotide displaying greater reduction in levels of the target RNA in neuronal cells over reduction in levels of the target RNA in glial cells is identified as being selective for neuronal cells. In some embodiments, the selectivity of the oligonucleotide for neuronal cells over glial cells is at least 1.5, 2, 2.5, 3, 3.5, 4, 4.5 or 5 or greater.

In some embodiments, the first candidate oligonucleotide and the second candidate oligonucleotide comprises a single stranded RNA or DNA, such as described herein.

In some embodiments, the first candidate oligonucleotide and the second candidate oligonucleotide comprises a double stranded nucleic acid (dsNA), wherein the dsNA comprises a ribonucleotide. In some embodiments, the first candidate oligonucleotide comprises a first sense strand and a first antisense strand, wherein the first sense strand and the second antisense strand form a first duplex and wherein the first antisense strand has the region of complementarity to the first target RNA expressed in a glial cell and is capable of reducing expression of first target RNA. In some embodiments, the second candidate oligonucleotide comprises a second sense strand and a second antisense strand, wherein the second sense strand and the second antisense strand form a second duplex and wherein the second antisense strand has the region of complementarity to the second target RNA expressed in the neuronal cell and is capable of reducing expression of target RNA.

In some embodiments, the first and second candidate oligonucleotides are modified with one or more targeting ligands. In some embodiments, the targeting ligand comprises one or more of a carbohydrate, amino sugar, cholesterol, lipid, peptide, or mixtures thereof. In some embodiments, the targeting ligand comprises one or more GalNAc moieties, as described in detail herein.

In some embodiments, wherein the first and second candidate oligonucleotides are administered to the nervous system of a subject, the measuring for determining the levels of target RNA in glial cells and neuronal cells is assessed by measuring the levels of the target RNA in glial cells and neuronal cells distributed throughout the nervous system or in localized regions of the nervous system. In some embodiments, the glial cell examined include, by way of example and not limitation, an astrocyte, oligodendrocyte, ependymal cell, microglial cell, Schwann cell, satellite cell, enteric glial cell, and combinations thereof. In some embodiments, neuronal cells include, by way of example and not limitation, motor neurons, sensory neurons, interneurons and combinations thereof.

In order that the invention described herein may be more fully understood, the following examples are set forth. The examples described in this application are offered to illustrate the methods, compositions, and systems provided herein and are not to be construed in any way as limiting their scope.

### EXAMPLES

### Example 1: Identification of Cell-Type-Specific Targeting siRNAs

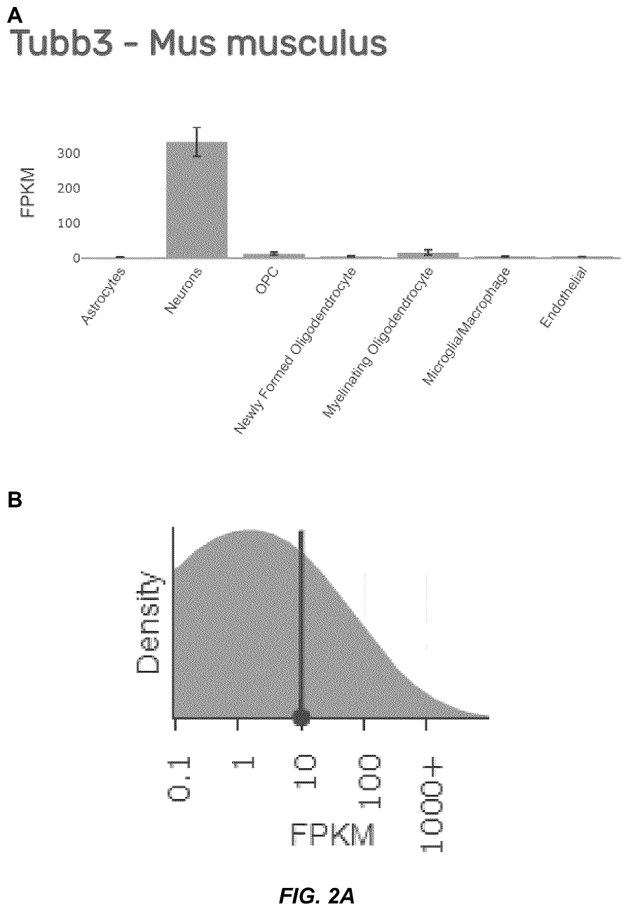

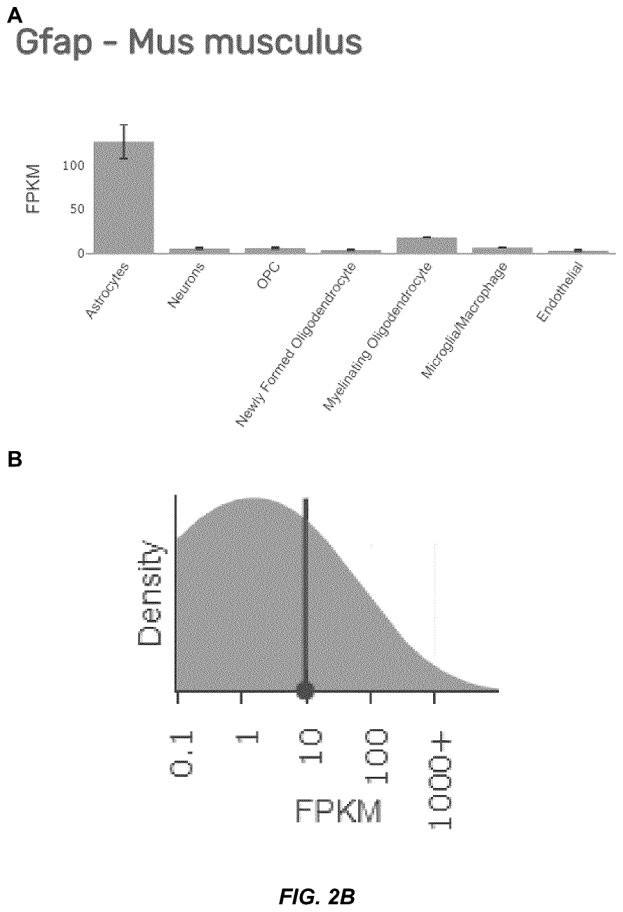

To assess the level of gene silencing in specific cell subpopulations, it was necessary to identify GalXC siRNAs targeting neuronal or astrocyte-specific genes. Tubb3 and Gfap mRNA were selected after a comprehensive review of available RNA-seq data. TUBB3, or Tubulin Beta 3 Class III, is a member of the beta tubulin family and involved in microtubule assembly. Tubb3 mRNA is predominantly expressed in neurons. GFAP, or Glial Fibrillary Acidic Protein, is a one of the major intermediate filament proteins in mature astrocytes and is often used as a marker to distinguish astrocytes from other non-neuronal cell types during development. The sequences and structures of the siRNA molecules prepared for the study are illustrated in FIG. 1. The expression of each target is therefore highly specific to neurons and astrocytes, respectively, as shown in FIG. 2A and FIG. 2B.

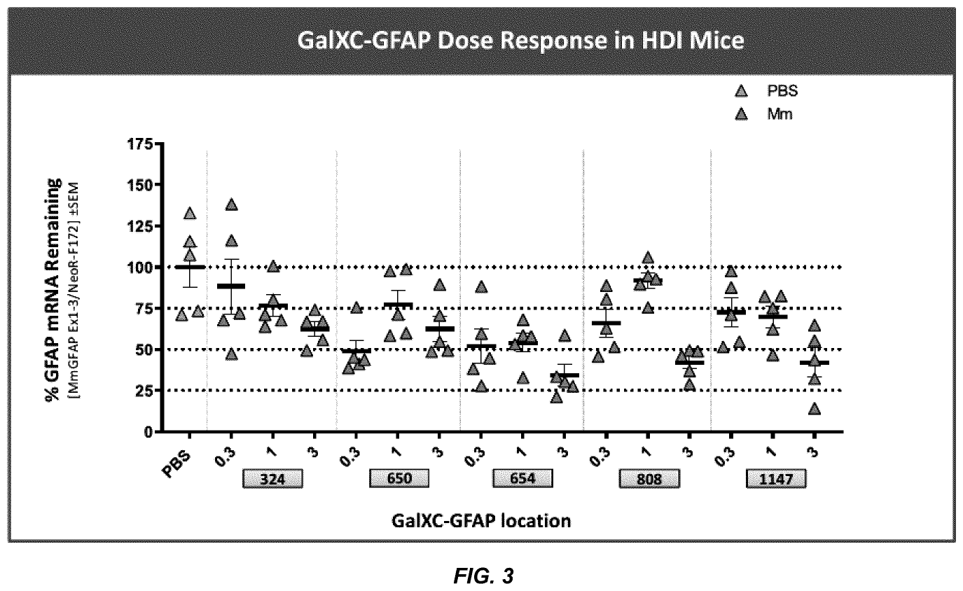

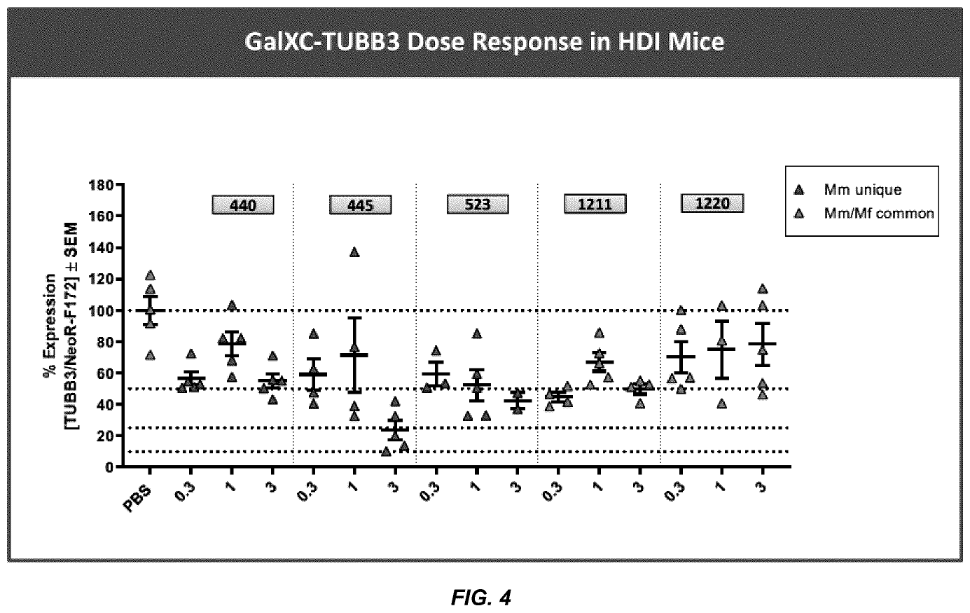

A GalXC-siRNA molecule targeting Gfap mRNA was generated by performing an in vivo screen (FIG. 3). A GalXC-siRNA molecule targeting Tubb3 mRNA was generated by performing a similar in vivo screen (FIG. 4). As an example, animals were injected subcutaneously with 0-3 mg/kg GalXC-TUBB3 on Day 1 and injected intravenously with a plasmid expressing Tubb3 mRNA on Day 4 (CD-1, female, n=5 per group). Tubb3 mRNA levels were analyzed in liver by RT-qPCR on Day 5. The top sequences from each screen had ED50 values in the ˜1 mg/kg range and were selected for evaluation in mouse CNS and PNS.

### Example 2: Selective Targeting of Neural Cell Subpopulations by GalXC siRNAs in the CNS

To GalXC-TUBB3 and GalXC-GFAP pharmacology was evaluated in neurons and astrocytes in the brain and spinal cord. An equivalent dose of either GalXC-TUBB3 or GalXC-GFAP was administered by bolus i.c.v. injection and Tubb3 and Gfap mRNA levels were measured after one week across the CNS. Both GalXC siRNAs have the same chemical composition and have the same relative ED50 as assessed by HDI in the liver. GalXC siRNAs are surprisingly capable of silencing Gfap mRNA to a much greater degree across all regions of the brain compared to equally potent GalXC siRNAs of similar chemical composition designed to target Tubb3 mRNA. The unexpected dramatic differences in potency observed between cell types across the entire brain and spinal cord clearly demonstrate that GalXC siRNA is capable of selective targeting of astrocytes after infusion into the CNS.

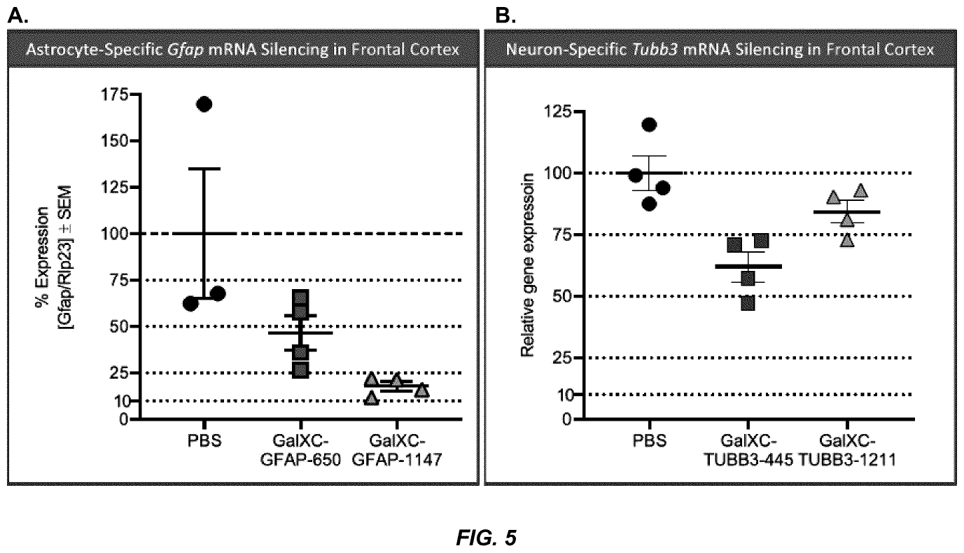

Frontal Cortex (FIG. 5): GalXC siRNAs targeting Gfap, a gene specifically expressed in astrocytes, are capable of up to 90% gene silencing in the frontal cortex following a single, bolus infusion of 500 μg into the right lateral ventricle (n=4 mice per group, CD1, female, 4-6 weeks of age). GalXC siRNAs targeting Tubb3, a gene specifically expressed in neurons, are only capable of ˜10-30% gene silencing following a single, bolus infusion of 500 μg into the right lateral ventricle (n=4 mice per group, CD1, female, 4-6 weeks of age).

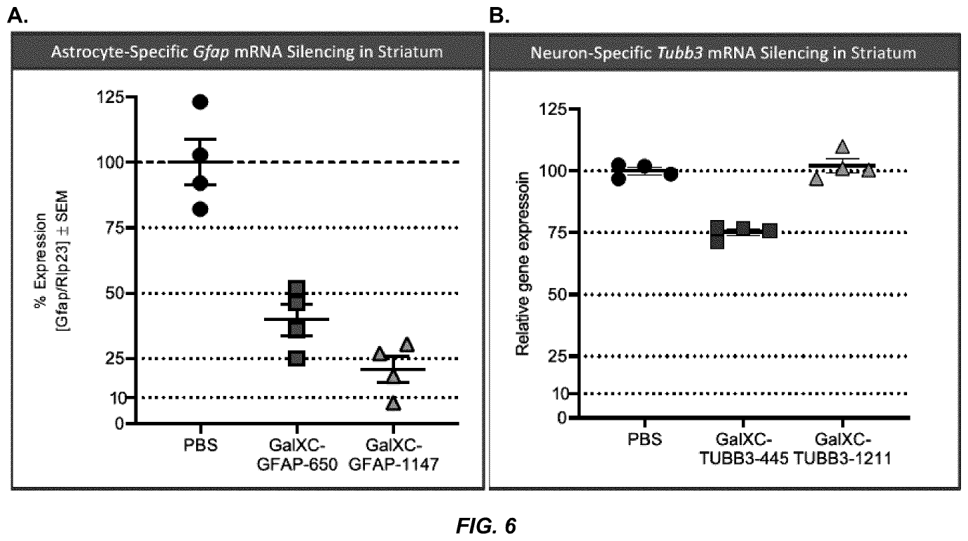

Striatum (FIG. 6): GalXC siRNAs targeting Gfap are surprisingly capable of up to 80% gene silencing in the striatum one week following a single, bolus infusion of 500 μg into the right lateral ventricle (n=4 mice per group, CD1, female, 4-6 weeks of age). On the other hand, GalXC siRNAs targeting Tubb3 are only capable of ˜0-25% gene silencing one week following a single, bolus infusion of 500 μg into the right lateral ventricle (n=4 mice per group, CD1, female, 4-6 weeks of age).

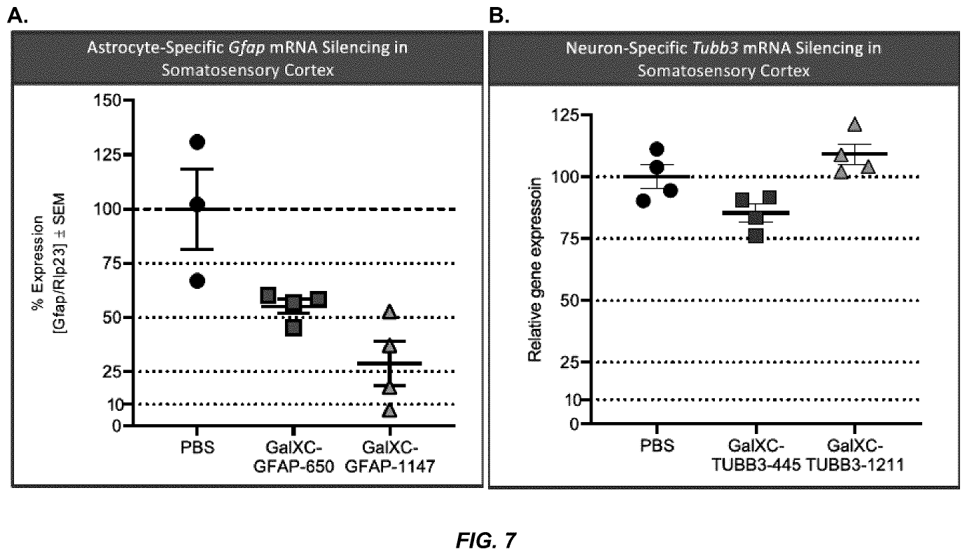

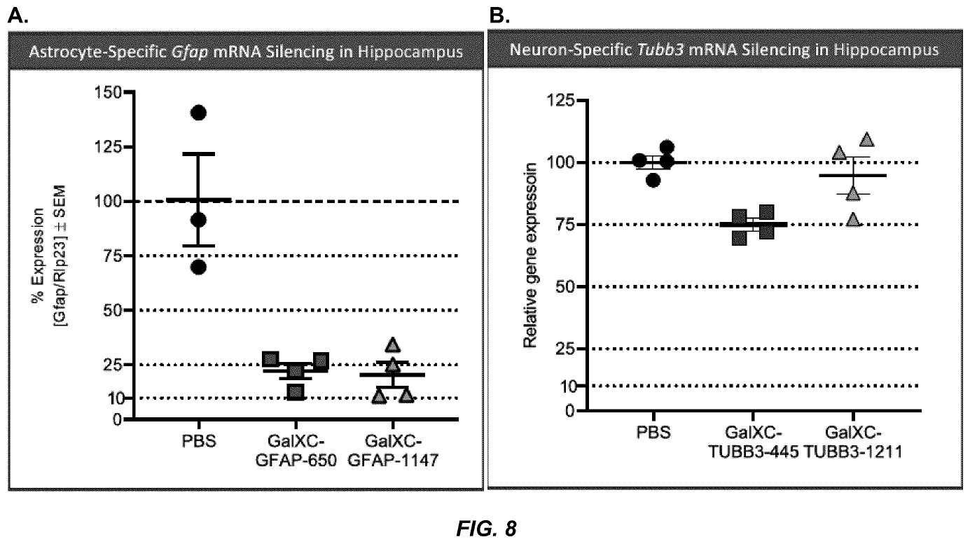

Somatosensory Cortex (FIG. 7): GalXC siRNAs targeting Gfap are capable of up to 75% gene silencing in the somatosensory cortex one week following a single, bolus infusion of 500 μg into the right lateral ventricle (n=4 mice per group, CD1, female, 4-6 weeks of age). Whereas, GalXC siRNAs targeting Tubb3 are only capable of ˜0-10% gene silencing one week following a single, bolus infusion of 500 μg into the right lateral ventricle (n=4 mice per group, CD1, female, 4-6 weeks of age).

Hippocampus (FIG. 8): GalXC siRNAs targeting Gfap are capable of up to 80% gene silencing in the hippocampus one week following a single, bolus infusion of 500 μg into the right lateral ventricle (n=4 mice per group, CD1, female, 4-6 weeks of age). GalXC siRNAs targeting Tubb3 are only capable of ˜0-25% gene silencing one week following a single, bolus infusion of 500 μg into the right lateral ventricle (n=4 mice per group, CD1, female, 4-6 weeks of age).

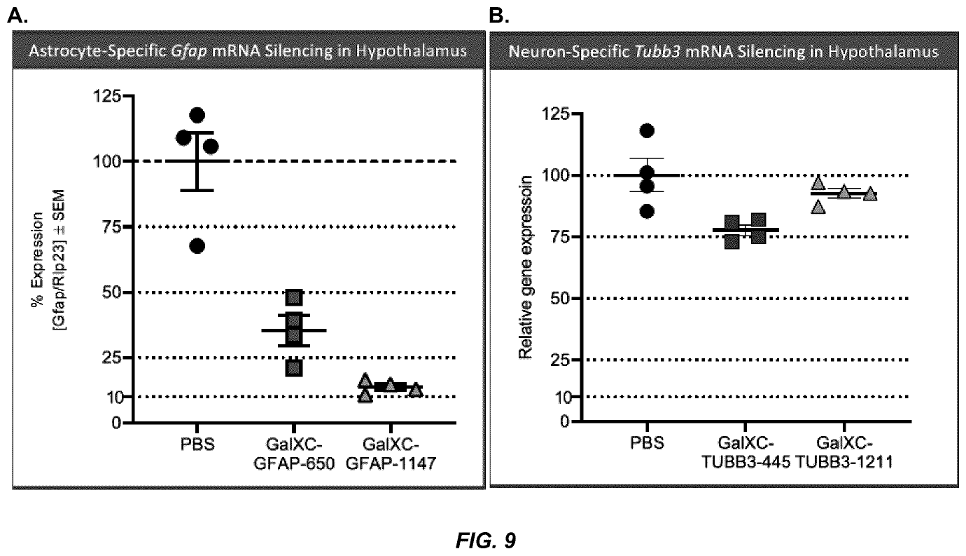

Hypothalamus (FIG. 9): GalXC siRNAs targeting Gfap are capable of up to 80% gene silencing in the hypothalamus one week following a single, bolus infusion of 500 μg into the right lateral ventricle (n=4 mice per group, CD1, female, 4-6 weeks of age). GalXC siRNAs targeting Tubb3 are only capable of ˜0-25% gene silencing one week following a single, bolus infusion of 500 μg into the right lateral ventricle (n=4 mice per group, CD1, female, 4-6 weeks of age).

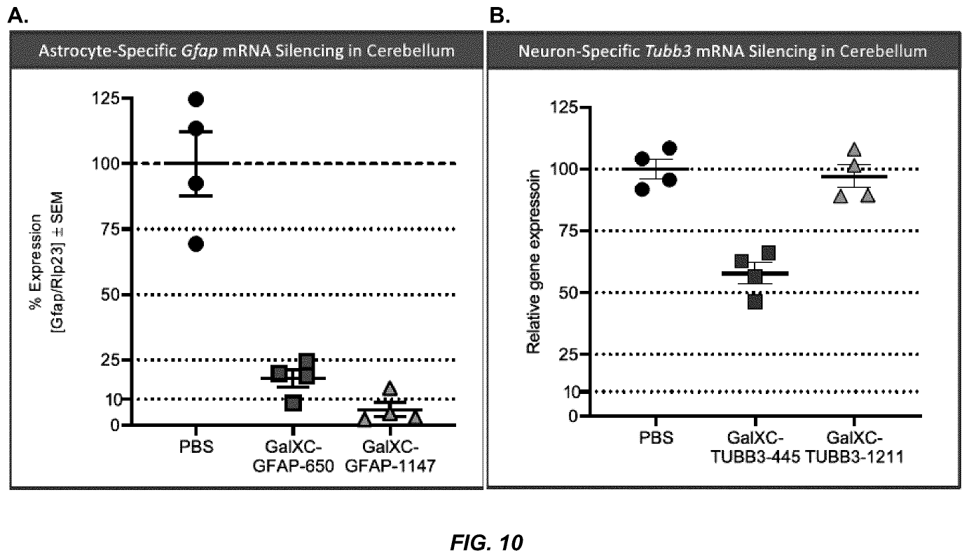

Cerebellum (FIG. 10): GalXC siRNAs targeting Gfap are capable of up to 95% gene silencing in the cerebellum one week following a single, bolus infusion of 500 μg into the right lateral ventricle (n=4 mice per group, CD1, female, 4-6 weeks of age). GalXC siRNAs targeting Tubb3 are only capable of ˜0-40% gene silencing one week following a single, bolus infusion of 500 μg into the right lateral ventricle (n=4 mice per group, CD1, female, 4-6 weeks of age).

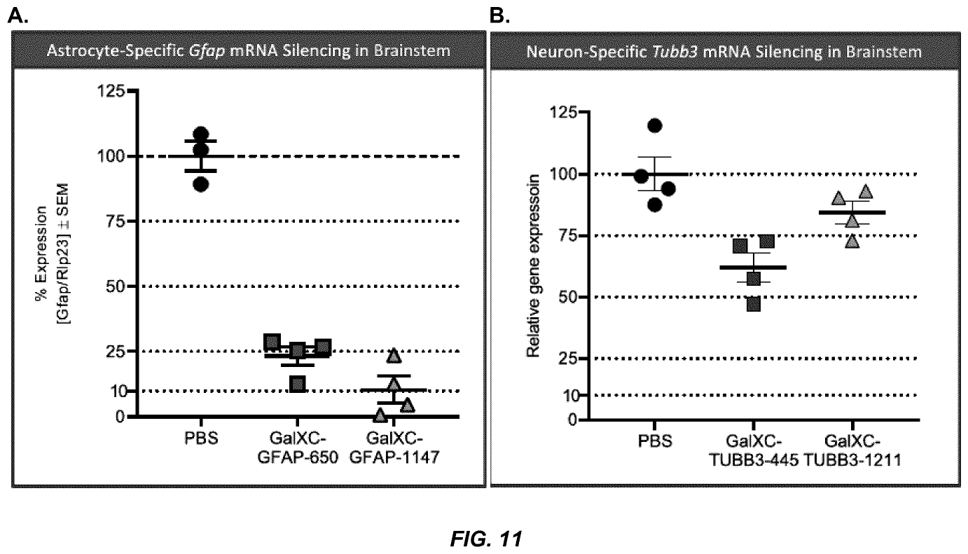

Brainstem (FIG. 11): GalXC siRNAs targeting Gfap are capable of up to 90% gene silencing in the brainstem one week following a single, bolus infusion of 500 μg into the right lateral ventricle (n=4 mice per group, CD1, female, 4-6 weeks of age). GalXC siRNAs targeting Tubb3 are only capable of ˜0-30% gene silencing one week following a single, bolus infusion of 500 μg into the right lateral ventricle (n=4 mice per group, CD1, female, 4-6 weeks of age).

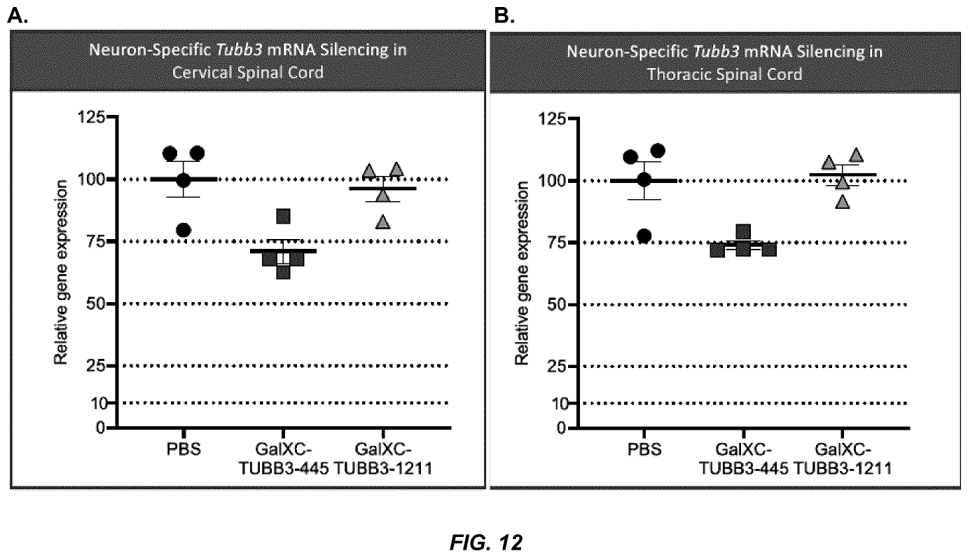

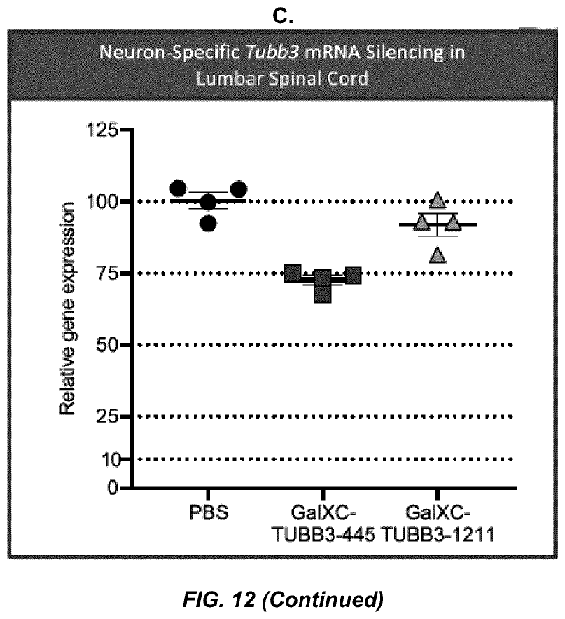

Spinal cord (FIG. 12): GalXC siRNAs targeting Tubb3, a gene specifically expressed in neurons, are capable of ˜0-25% gene silencing one week following a single, bolus infusion of 500 μg into the right lateral ventricle (n=4 mice per group, CD1, female, 4-6 weeks of age). In this chemical configuration, no gene silencing would be expected in neurons in the spinal cord (motor neurons, interneurons, etc.) following a bolus infusion into the cerebral spinal fluid, even if the target gene had expression in both glial and neuronal cell types. This alleviates any concern about neuronal toxicity or viability when treating diseases of the glia.

### Example 3: Selective Targeting of Neural Cell Subpopulations by siRNAs Lacking GalNAc Targeting Ligands

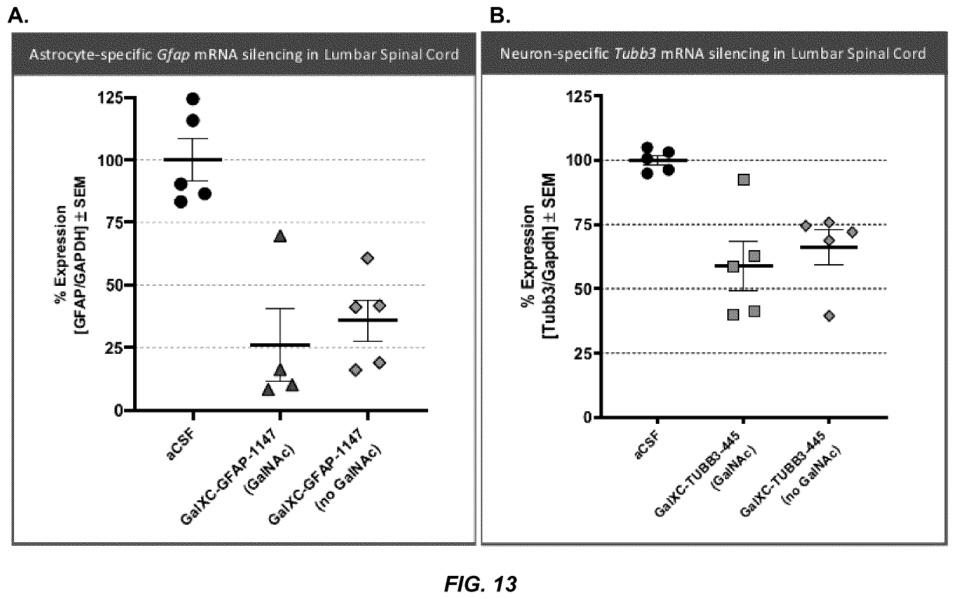

This study examined the role of GaNAc residues in targeting siRNAs to neurons and glial cells. In panel A of FIG. 13, GalXC-GFAP-1147 (GalNAc) is standard GalXC chemistry with three attached GalNAc moieties while GalXC-GFAP-1147 (no GalNAc) is an analogous molecule with identical chemistry but lacking the three conjugated GalNAc sugars. The GFAP specific siRNAs were administered as a single, bolus infusion of 500 μg by lumbar intrathecal injection into the subarachnoid space (n=5 mice per group, C57BL/6, female, 4-6 weeks of age). The GalXC siRNAs targeting Gfap mRNA were capable of up to 70-75% gene silencing in astrocytes in the lumbar spinal cord one week following administration, demonstrating that the absence GalNAc moieties on the siRNAs does not reduce the ability of these oligonucleotides to silence their gene target in the murine brain.

In panel B of FIG. 13, GalXC-TUBB3-445 (GalNAc) is standard GalXC chemistry with three attached GalNAc moieties while GalXC-TUBB3-445 (no GalNAc) is an analogous molecule with identical chemistry but lacking the three conjugated GalNAc sugars. The TUBB specific siRNAs were administered as a single, bolus infusion of 500 μg by lumbar intrathecal injection into the subarachnoid space (n=5 mice per group, C57BL/6, female, 4-6 weeks of age). GalXC siRNAs targeting Tubb3 mRNA were capable of up to 30-35% gene silencing in neurons in the lumbar spinal cord one week following administration, demonstrating that the absence of GalNAc moieties does not reduce the ability of these oligonucleotides to silence their gene target in murine brain.

Maintenance of the differential targeting of the siRNAs to astrocytes compared to neurons indicates that the siRNAs can be preferentially delivered to glial cells with or without the GalNAc moieties.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counter part of an RNA nucleotide) and/or one or more modified nucleotides and/or one or more modified internucleotide linkages and/or one or more other modification compared with the specified sequence while retaining essentially same or similar complementary properties as the specified sequence.

It should also be understood that the use of the terms “a” and “an” and “the” and similar referents in the context of describing the invention arc to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted.

Furthermore, recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of all examples, or exemplary language (e.g., “such as”) provided herein, is intended merely to better illuminate the invention and does not pose a limitation on the scope of the invention unless otherwise claimed.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. A method of selectively delivering an interfering oligonucleotide to glial cells, comprising:
contacting a glial cell with an oligonucleotide comprising a region of complementarity to a target RNA expressed in the glial cell, wherein the oligonucleotide is capable of reducing expression of the target RNA, wherein the oligonucleotide comprises a sense strand and an antisense strand, wherein the sense strand and the antisense strand form a duplex, and the antisense strand comprises a region of complementarity to a target RNA expressed in the glial cell and is capable of reducing expression of the target RNA.

2. (canceled)

10. The method of claim 1, wherein the oligonucleotide comprises a dsRNA, wherein the sense strand is 15 to 40 nucleotides in length, wherein the antisense strand is 19 to 27 nucleotides in length, and wherein the sense strand and the antisense strand form a duplex of at least 12 nucleotides in length; and wherein the antisense strand has a 3′ overhang, and wherein the 3′-overhang is 2 nucleotides in length.

11.-16. (canceled)

17. The method of claim 1, wherein the oligonucleotide further comprises a stem-loop sequence comprising sequence regions S1-L-S2, wherein S1 is complementary to S2, and wherein L is a loop that forms between S1 and S2 when S1 and S2 form a duplex, and wherein the stem-loop sequence is attached to the sense strand at its 3′-end; wherein the loop L is four nucleotides in length; wherein the oligonucleotide further comprises one or more targeting ligands, wherein the targeting ligand is present on the loop L of the stem-loop sequence; and the targeting ligand is a GalNAc moiety.

18.-21. (canceled)

22. The method of claim 1, wherein the oligonucleotide comprises a means for increasing resistance of the oligonucleotide to a phosphatase and/or nuclease, increasing hybridization efficiency, and/or enhancing in vivo stability.

23. The method of claim 1, wherein the oligonucleotide comprises at least one modified nucleotide.

24. The method of claim 1, wherein the glial cell is an astrocyte, oligodendrocyte, ependymal cell, microglial cell, Schwann cell, satellite cell, enteric glial cell, or mixtures thereof.

25. The method of claim 1, wherein the glial cell is present in the nervous system of a subject, optionally wherein the glial cell is present in the central nervous system of a subject, or in the peripheral nervous system of a subject.

26. (canceled)

27. The method of claim 1, wherein the glial cell is present in the frontal cortex, striatum, somatosensory cortex, hippocampus, hypothalamus, cerebellum, brainstem, and/or spinal cord, optionally wherein the glial cell is present in the cervical spinal cord, thoracic spinal cord, and/or lumbar spinal cord.

28.-30. (canceled)

31. The method of claim 1, wherein the target RNA is specifically expressed in the glial cell compared to a neuronal cell; and wherein the selectivity is at least about 1.5 for glial cells over neuronal cells.

32. The method of claim 1, wherein the region of complementarity is to an exon in the target RNA, to a 5′ untranslated region of the target RNA, to a 3′ untranslated region of the target RNA, or to an allele specific sequence of the target RNA.

33.-37. (canceled)

38. The method of claim 1, wherein the target RNA is an RNA expressed from: GFAP gene, PSAP gene, PMP22 gene, LMNB1 gene, APP gene, TAU (MAPT) gene, SOD1 gene, C9orf72 gene, HTT gene, SNCA or ASYN gene, LRRK2 gene, ADK gene, TNFα gene, ERK5/MAPK7 gene, IL-1R2 gene, CD49d gene, IGF-1 gene, EGF gene, TGF-β gene, VEGF gene, TDP-43 gene, CD38 gene, ATXN2 gene, ATXN3 gene, ATXN7 gene, or EGR2 gene.

39. The method of claim 1, wherein the glial cell is a glial cancer cell and the target RNA is an RNA with increased expression in the glial cancer cell, optionally wherein the glial cancer cell is a glioblastoma.

40. (canceled)

41. The method of claim 39, wherein the glial cancer cell is a glioblastoma and wherein the target RNA is RNA expressed in the glial cancer cell from the IGF-1 gene, EGF gene, TGF-β gene, or VEGF gene.

42.-84 (canceled)

85. A method of screening for an oligonucleotide that selectively reduces levels of a target RNA expressed in glial cells or neuronal cells, comprising:
contacting a glial cell and a neuronal cell with a candidate oligonucleotide, or administering a candidate oligonucleotide to a nervous system of a subject, wherein the candidate oligonucleotide comprises a region of complementarity to a target RNA expressed in both the glial cell and neuronal cell; and
measuring the levels of target RNA in glial cells and neuronal cells to determine any differential effect on levels of target RNA expression.

86. (canceled)

87. (canceled)

88. The method of claim 85, wherein the oligonucleotide comprises a double stranded nucleic acid (dsNA), wherein the dsNA comprises a ribonucleotide; wherein the oligonucleotide comprises a sense strand and an antisense strand, wherein the sense strand and the antisense strand form a duplex, and the antisense strand has the region of complementarity to the target RNA, and wherein the oligonucleotide is capable of reducing levels of target RNA expression.

89. (canceled)

90. The method of claim 85, wherein the oligonucleotide further comprises one or more targeting ligands, wherein the targeting ligand comprises a GalNAc moiety.

91. (canceled)

92. (canceled)

93. The method of claim 85, wherein the measuring comprises determining the levels of target RNA distributed throughout glial cells and neuronal cells of the nervous system.

94. The method of claim 85, wherein the glial cell is an astrocyte, oligodendrocyte, ependymal cell, microglial cell, Schwann cell, satellite cell, or enteric glial cell.

95.-107. (canceled)

108. A double stranded oligonucleotide selected from Table A.


SEQUENCE LISTING (13 sequences)

SEQ ID NO: 1 - RNA, 36 nt, Artificial Sequence
  source: /note=“Description of Artificial Sequence Synthetic oligonucleotide”
uuggagagaaagguugaauagcagccgaaaggcugc

SEQ ID NO: 2 - RNA, 22 nt, Artificial Sequence
  source: /note=“Description of Artificial Sequence Synthetic oligonucleotide”
uauucaaccuuucucuccaagg

SEQ ID NO: 3 - RNA, 36 nt, Artificial Sequence
  source: /note=“Description of Artificial Sequence Synthetic oligonucleotide”
aggaucuacucaacguuaaagcagccgaaaggcugc

SEQ ID NO: 4 - RNA, 22 nt, Artificial Sequence
  source: /note=“Description of Artificial Sequence Synthetic oligonucleotide”
uuuaacguugaguagauccugg

SEQ ID NO: 5 - RNA, 36 nt, Artificial Sequence
  source: /note=“Description of Artificial Sequence Synthetic oligonucleotide”
agugugagaauugugacugagcagccgaaaggcugc

SEQ ID NO: 6 - RNA, 22 nt, Artificial Sequence
  source: /note=“Description of Artificial Sequence Synthetic oligonucleotide”
ucagucacaauucucacacugg

SEQ ID NO: 7 - RNA, 36 nt, Artificial Sequence
  source: /note=“Description of Artificial Sequence Synthetic oligonucleotide”
agaacagcagcuacuucguagcagccgaaaggcugc

SEQ ID NO: 8 - RNA, 22 nt, Artificial Sequence
  source: /note=“Description of Artificial Sequence Synthetic oligonucleotide”
uacgaaguagcugcuguucugg

SEQ ID NO: 9 - RNA, 58 nt, Artificial Sequence
  source: /note=“Description of Artificial Sequence Synthetic oligonucleotide”
uuggagagaaagguugaauagcagccgaaaggcugcuauucaaccuuucucuccaagg

SEQ ID NO: 10 - RNA, 58 nt, Artificial Sequence
  source: /note=“Description of Artificial Sequence Synthetic oligonucleotide”
aggaucuacucaacguuaaagcagccgaaaggcugcuuuaacguugaguagauccugg

SEQ ID NO: 11 - RNA, 58 nt, Artificial Sequence
  source: /note=“Description of Artificial Sequence Synthetic oligonucleotide”
agugugagaauugugacugagcagccgaaaggcugcucagucacaauucucacacugg

SEQ ID NO: 12 - RNA, 58 nt, Artificial Sequence
  source: /note=“Description of Artificial Sequence Synthetic oligonucleotide”
agaacagcagcuacuucguagcagccgaaaggcugcuacgaaguagcugcuguucugg

SEQ ID NO: 13 - protein, 5 aa, Artificial Sequence
  source: /note=“Description of Artificial Sequence Synthetic peptide”
CREKA